(19) United States
(12) Patent Application Publication
(10) Pub. No.: US 2015/0079012 A1
(43) Pub. Date: Mar. 19, 2015

(54) SILK FIBROIN-BASED PERSONAL CARE COMPOSITIONS

(71) Applicant: TRUSTEES OF TUFTS COLLEGE, Medford, MA (US)

(72) Inventors: Evangelia Bellas, Somerville, MA (US); Amanda Baryshyan, Somerville, MA (US); Lindsay Wray, Medford, MA (US); David L. Kaplan, Concord, MA (US)

(21) Appl. No.: 14/395,396

(22) PCT Filed: Apr. 22, 2013
(86) PCT No.: PCT/US13/37613

§ 371 Date: Oct. 17, 2014

Related U.S. Application Data
(60) Provisional application No. 61/636,061, filed on Apr. 20, 2012.

(51) Int. Cl. A61K 8/64; A23L 1/305; A61Q 5/02; A61K 8/55; A61K 47/10; A61K 47/46

(52) U.S. Cl. CPC A61K 8/64; A61K 47/10; A61K 47/46; A61Q 5/02; A61K 8/553; A23L 1/305; A61K 2800/10
USPC 424/62; 514/21.2; 510/119; 510/122

(57) ABSTRACT

Provided herein relates to silk fibroin-based compositions comprising non-hydrolyzed silk fibroin and a humectant agent and methods of making the same. In some embodiments, the silk fibroin-based compositions can be formulated for personal care products, e.g., but not limited to, cosmetic products, skin care products, body care products, and/or hair products. In some embodiments, the silk fibroin-based compositions can be formulated for food applications.

### CROSS REFERENCE TO RELATED APPLICATIONS

This application claims the benefit under 35 U.S.C. §119(e) of U.S. Provisional Application No. 61/636,061 filed Apr. 20, 2012, the content of which is incorporated herein by reference in its entirety.

### GOVERNMENT SUPPORT

This invention was made with government support under grant EB002520 awarded by the National Institutes of Health (NIH). The government has certain rights in the invention.

### FIELD OF THE DISCLOSURE

The present invention generally relates to silk fibroin-based compositions and methods of making the same. In particular, the silk fibroin-based compositions described herein can be adapted to form personal care products such as cosmetics, skincare products, body care products, and/or hair products. In some embodiments, the silk fibroin-based compositions can be adapted for food applications.

### BACKGROUND OF THE DISCLOSURE

The majority of existing skin and hair care products generally contain synthetic ingredients that can be harmful to human health and/or the environment. When these products are applied to the body, these synthetic ingredients can be absorbed through the skin, ingested or inhaled. Additionally, the products can be washed off into the wastewater, where they can affect the environment. Since skin and hair care products are typically used on a daily basis, harmful synthetic ingredients can accumulate in the body and thus cause adverse health effects over time. In particular, scientific studies have shown that certain synthetic components found in beauty products are believed to be linked to fatal diseases such as cancer, cardiovascular and neurological dysfunction, and birth defects (Darbre et al., 2004; Carruthers et al., 2005).

One promising option for overcoming this problem is to replace harmful, synthetic materials with safe, all-natural ingredients. Unfortunately, the task of formulating all-natural skin and hair care products is non-trivial. The primary reason for the use of synthetic components in beauty products is that they are inexpensive to reproducibly manufacture and can be easily incorporated into products to yield a desirable look and feel. In contrast, natural components are extracted from raw sources, which can lead to batch inconsistencies. Furthermore, natural components often do not replicate the aesthetic look and/or feel that consumers have been accustomed to when using synthetic products. In recent years, there has been a growing demand for beauty products formulated with natural components. However, many of these products fail to be 100% all-natural and continue to use synthetic components in their formulations. Therefore, there is still an unmet need for all-natural beauty products.

### SUMMARY

Embodiments of various aspects provided herein generally relate to compositions comprising non-hydrolyzed silk fibroin and a humectant, as well as methods of making and using the same. While some existing personal care products (e.g., skincare or hair products) that claim to have silk fibroin in the formulation, these products use silk fibroin in a hydrolyzed form. That is, the intact silk fibroin protein has been hydrolyzed or broken down into a mixture of small fragments (e.g., less than 20 kDa or lower) and/or constituent amino acids, which become no different from glycine, alanine, and serine derived from non-silk sources. In contrast, the compositions described herein comprise silk fibroin in a non-hydrolyzed form. Silk fibroin in a non-hydrolyzed or intact protein form has many desirable qualities, e.g., but not limited to, strength, luster, smooth feel, and its ability to absorb moisture that can be beneficial, e.g., to topical hair and skin care. However, once the silk fibroin protein is hydrolyzed, these desirable qualities are lost because they are inherent to the tertiary and quaternary structure of the non-hydrolyzed silk fibroin molecules.

While hydrolyzed silk amino acids exist as an aqueous solution that is relatively easy to work into skin and hair care formulation, non-hydrolyzed or intact silk fibroin solution needs to be prepared in a highly-regulated manner in order to preserve the tertiary and quaternary structure of the protein. Further, a non-hydrolyzed silk fibroin solution can become a brittle gel after 1-2 months of storage at about 4° C., and it can shear-thicken and form precipitates when rubbed on the skin. To overcome these challenges, the inventors have discovered inter alia a novel composition and method for stabilizing non-hydrolyzed silk fibroin protein so that it can be incorporated into beauty products that can provide long-lasting moisture and protection for skin and hair as well as a pleasant aesthetic. In one embodiment, the inventors discovered that the combination of a non-hydrolyzed silk fibroin solution and a humectant (e.g., a glycerin) can produce a composition with a gel-like consistency that is translucent and flowable, which is distinctly different from the brittle gel formed from the silk fibroin solution alone. In particular, when the volume ratio of glycerin to non-hydrolyzed silk fibroin solution is greater than 1:1 (e.g., about 2:1 to about 8:1), the interaction of glycerin and silk fibroin surprisingly provides a gel composition that is stable at room temperature for an extended period of time, e.g., at least about 12 months and does not form precipitation of the protein when sheared on the skin. Accordingly, provided herein are novel compositions and methods of stabilizing non-hydrolyzed silk fibroin proteins, which can be versatile in various applications, e.g., skin and hair products.

In one aspect, a silk fibroin-based composition comprising non-hydrolyzed silk fibroin and a humectant agent is provided herein. In some embodiments, the non-hydrolyzed silk fibroin can have an average molecular weight of at least about 20 kDa or higher. In some embodiments, the non-hydrolyzed silk can have an average molecular weight of at least about 30 kDa or higher. In some embodiments, at least a portion of the non-hydrolyzed silk fibroin can maintain its tertiary and/or quaternary structures.

Any agent or substance that can absorb moisture from the surrounding, reduce moisture loss from non-hydrolyzed silk fibroin or the composition described herein, and/or promote non-hydrolyzed silk fibroin or the composition described herein to retain moisture can be employed as a humectant agent in the compositions described herein. Examples of the humectant agents include, but are not limited to, phospholipid (e.g., lecithin, panthenol (pro-vitamin B5), glycerin (glycerol)), aloe vera, sorbitol, urea, alpha-hydroxy acids, sodium pyroglutamate, N-acetyl-ethanolamine, sodium lactate, isopropanol, polyalkylene glycols (e.g., ethylene glycol, propylene glycol, hexylene glycol, butylene glycol, dipropylene glycol, triethylene glycol), 1,3-propanediol, polyethylene glycols, synthetic alcohols (e.g., glyceryl coconate, hydroxystearate, myristate, oleate), and any combinations thereof. In some embodiments, the humectant agent used in the composition can comprise phospholipid (e.g., but not limited to, lecithin, panthenol (pro-vitamin B5), glycerin), aloe vera, sorbital, alpha-hydroxy acids, or any combinations thereof.

Amounts of humectant agents present in the composition of any aspects described herein can be optimized to yield a composition that (1) is shelf-stable, e.g., for at least about 24 hours or longer (e.g., as along as 1 year or even longer) at about room temperature or higher; and/or (2) does not shear-thicken or form precipitates when sheared. For example, in some embodiments of this aspect and other aspects described herein, the humectant agent can be present in an amount of about 0.1% (w/v or w/w) to about 99.9% (w/v or w/w). In some embodiments, the humectant agent can be present in an amount of at least about 50% (w/v or w/w), at least about 60% (w/v or w/w), at least about 70% (w/v or w/w), at least about 80% (w/v or w/w), at least about 90% (w/v or w/w) or higher (excluding 100%). In some embodiments, the humectant agent can be present in an amount of about 50% (w/v or w/w) to about 99% (w/v or w/w).

In one embodiment of this aspect and other aspects described herein, the humectant agent comprises glycerin. While glycerin (also known as glycerol) has been previously used in a silk fibroin composition (e.g., described in the International Patent Application No. WO 2010/042798, the content of which is incorporated herein by reference), it was mainly incorporated as a plasticizer, at a glycerin: silk weight ratio up to 1:1, to make silk materials (e.g., a silk film) more flexible. The '798 patent application does not describe a composition comprising glycerin and silk fibroin in a weight ratio greater than 1:1, or a composition comprising silk fibroin and glycerin in an amount greater than 50% (w/w) as described herein. The inventors have discovered inter alia that the gels formed from a mixture of glycerin and silk fibroin in a ratio greater than 1:1 (e.g., glycerin: silk ration=˜4:1) differ from the silk fibroin-based materials described in the '798 patent application (i.e., a silk fibroin-based material formed from the same components but with a ratio of glycerin to silk fibroin no more than 1:1). For example, unlike flexible silk fibroin films described in the '798 patent applications, the compositions described herein have a gel-consistency with silk fibroin maintained in dominant random-coil content, which likely contributes to its flowability. Accordingly, in some embodiments of this aspect and other aspects described herein, glycerin can be present in an amount of at least about 50% (w/v or w/w), at least about 60% (w/v or w/w), at least about 70% (w/v or w/w), at least about 80% (w/v or w/w), at least about 90% (w/v or w/w) or higher (excluding 100%). In some embodiments, glycerin can be present in an amount of about 50% (w/v or w/w) to about 99% (w/v or w/w).

In some embodiments of this aspect and other aspects described herein, the non-hydrolyzed silk fibroin can be present in an amount of about 1% (w/v or w/w) to about 30% (w/v or w/w), or about 1% (w/v or w/w) to about 10% (w/v or w/w).

In some embodiments of this aspect and other aspects described herein, the non-hydrolyzed silk fibroin and the humectant agent can be present in any ratio, provided that the amount of the humectant agent is more than that of the non-hydrolyzed silk fibroin. For example, the non-hydrolyzed silk fibroin and the humectant agent can be present in a volume ratio of about 1:1.1 to about 1:100, or about 1:2 to about 1:100, or about 1:2 to about 1:10, or about 1:2 to about 1:8. Stated another way, the non-hydrolyzed silk fibroin and the humectant agent can be present in a weight/mass ratio of about 1:1.1 to 1:1000, about 1:5 to about 1:750, or about 1:10 to about 1:500.

In some embodiments of this aspect and other aspects described herein, the volume or weight ratio of the non-hydrolyzed silk fibroin to the humectant agent can be adjusted to yield a translucent composition. Accordingly, in some embodiments, the composition described herein can be translucent.

In some embodiments of this aspect and other aspects described herein, the volume or weight ratio of the non-hydrolyzed silk fibroin to the humectant can be adjusted to yield a flowable or spreadable composition. Accordingly, in some embodiments, the composition described herein can be flowable or readily spreadable on a surface.

In some embodiments of this aspect and other aspects described herein, the volume or weight ratio of the non-hydrolyzed silk fibroin to the humectant can be adjusted to yield a composition that does not shear-thicken or precipitate when exposed to shear. Accordingly, in some embodiments, the composition described herein can be resistant to shear-thickening or does not readily form precipitates or particulates when exposed to shear. In some embodiments, the composition described herein can be shear-thinning when exposed to shear. In some embodiments, the composition described herein can substantially maintain their viscosity or rheology when exposed to shear.

The compositions described herein are distinct from a composition comprising non-hydrolyzed silk fibroin without a humectant agent. For example, the inventors have discovered that when non-hydrolyzed silk fibroin protein forms a gel in the absence of a humectant, the resulting gel contains a prominent beta-sheet content, as opposed to more random coil conformation maintained in the silk fibroin-based gels comprising a humectant. Thus, in some embodiments, the composition described herein can have a larger amount of silk fibroin in random coil conformation, as compared to a composition comprising the non-hydrolyzed silk fibroin in the absence of the humectant agent. For example, the amount of silk fibroin in random coil conformation can be at least about 10% or more, larger than when the humectant agent is absent.

In some embodiments, the composition described herein can have a smaller amount of silk fibroin in beta-sheet conformation, as compared to a composition comprising the non-hydrolyzed silk fibroin in the absence of the humectant agent. For example, the amount of silk fibroin in beta sheet conformation can be at least about 10% or more, smaller than when the humectant agent is absent.

In some embodiments, the composition described herein does not have a peak in the amide II region (e.g., between ˜1475 cm⁻¹ and ˜1560 cm⁻¹), as determined in a Fourier Transform Infrared Spectroscopy (FTIR) spectrum.

In some embodiments, the composition described herein can be used to form an aqueous-based phase, which can then be mixed with an oil-based phase or a wax-based phase to form an emulsion. The inventors have discovered that when an aqueous mixture of a humectant (e.g., glycerin) and non-hydrolyzed silk fibroin was added to an oil-based solution, the non-hydrolyzed silk fibroin plays a role in emulsifying the oil and aqueous phases. For example, addition of soy lecithin alone is not a sufficient emulsifier to emulsify the aqueous and oil phases. The use of hydrolyzed silk fibroin or water in place of non-hydrolyzed silk fibroin resulted in phase separation, even in the presence of soy lecithin. Thus, soy lecithin alone is not sufficient to stabilize an emulsion, e.g., an oil-in-water emulsion where it contains hydrolyzed silk, but not non-hydrolyzed silk fibroin. Only when non-hydrolyzed silk fibroin was mixed with the aqueous and oil phases did the ingredients form a single-phase gel. Thus, in some embodiments, non-hydrolyzed silk fibroin can act as an emulsifying agent in the compositions described herein and/or aid in an emulsification process, allowing aqueous and oil phases to mix and form a single-phase gel (e.g., a flowable single-phase gel). This has not been previously reported for non-hydrolyzed silk fibroin, and, without wishing to be bound by theory, the ability of non-hydrolyzed silk fibroin to act as an emulsifying agent can be in part due to the protein having both hydrophilic and hydrophobic regions.

Accordingly, in another aspect, an emulsion composition comprising non-hydrolyzed silk fibroin is also provided herein. The emulsion composition comprises an aqueous-based phase, and an oil-based phase and/or a wax-based phase, wherein the aqueous-based phase comprises non-hydrolyzed silk fibroin and a humectant agent described herein. In some embodiments, the emulsion composition can further comprise any natural or synthetic emulsifier known in the art, including, for example, liquid soy lecithin, solid soy lecithin, honey, beeswax, cetyl alcohol, or any combinations thereof.

In some embodiments, the oil-based phase can comprise a carrier oil. For example, a carrier oil can be a synthetic or natural oil derived from seeds, nuts, fruits, flowers, plant-based materials, or any combinations thereof. In some embodiments, the wax-based phase can comprise, e.g., but not limited to, butters, wax, paraffin wax, paraffin oil, petrolatum, or any combinations thereof.

While the humectant agent can be present in any appropriate amount in the aqueous-based phase, in some embodiments, the humectant agent can be present in an amount of at least about 50% (w/v or w/w), at least about 60% (w/v or w/w), at least about 70% (w/v or w/w), at least about 80% (w/v or w/w), at least about 90% (w/v or w/w) or higher (excluding 100%) of the aqueous-based phase. In some embodiments, the humectant agent can be present in an amount of about 50% (w/v or w/w) to about 99% (w/v or w/w) of the aqueous-based phase.

In some embodiments where glycerin is a selected humectant agent, glycerin can be present in an amount of at least about 50% (w/v or w/w), at least about 60% (w/v or w/w), at least about 70% (w/v or w/w), at least about 80% (w/v or w/w), at least about 90% (w/v or w/w) or higher (excluding 100%) of the aqueous-based phase. In some embodiments, glycerin can be present in an amount of about 50% (w/v or w/w) to about 99% (w/v or w/w) of the aqueous-based phase.

In some embodiments, the non-hydrolyzed silk fibroin can be present in an amount of about 1% (w/v or w/w) to about 30% (w/v or w/w), or about 1% (w/v or w/w) to about 10% (w/v or w/w) of the aqueous-based phase.

In some embodiments, the non-hydrolyzed silk fibroin and the humectant agent in the aqueous-based phase can be in any ratio, provided that the amount of the humectant agent is more than that of the non-hydrolyzed silk fibroin. For example, in the aqueous-based phase, the non-hydrolyzed silk fibroin and the humectant agent can be present in a volume ratio of about 1:1.1 to about 1:100, or about 1:2 to about 1:100, or about 1:2 to about 1:10, or about 1:2 to about 1:8. Stated another way, the non-hydrolyzed silk fibroin and the humectant agent can be present in a weight/mass ratio of about 1:1.1 to 1:1000, about 1:5 to about 1:750, or about 1:10 to about 1:500 in the aqueous-based phase.

The ratio of the aqueous-based phase to the oil-based phase can vary to form different types of emulsion compositions, e.g., water-in-oil compositions or oil-in-water compositions. Accordingly, the ratio of the aqueous-based phase to the oil-based phase can vary from about 1:100 to about 100:1.

In some embodiments, the emulsion composition can comprise at least one or more additives. For example, at least one additive can be present in the aqueous-based phase. Additionally or alternatively, the oil-based phase can comprise at least one or more additives.

Compositions of various aspects described herein are versatile and can be adapted for various applications, e.g., personal care, skin care, body care, wound healing, or any applications where a flowable or spreadable composition is desirable. Accordingly, in some embodiments, the silk fibroin-based composition and/or the emulsion composition described herein can further comprise an additive, e.g., to suit the need of an application. Examples of an additive include, but are not limited to, aqueous-soluble components, oil-soluble components, wax-soluble components, essential oils, emulsifiers, surfactants, flavors, fragrance, nutraceuticals, vitamins, therapeutic agents, cosmeceuticals, preservatives, botanical extracts or distillates, antioxidants, anti-bacterial agents, anti-inflammatory agents, cosmetically-acceptable agents, skin-lightening agents, skin-evening agents, anti-reddening agents, viscosity modifiers, emollients, active agents, nano- or micro-particles, photothermal elements (e.g., gold nanoparticles), wound healing-promoting agents, and any combinations thereof.

One of skill in the art can determine appropriate additives and amounts thereof based on different applications. In one embodiment, the composition can be formulated for use in a personal care product (e.g., a cosmetic, skincare, body care, or hair product). In one embodiment, the composition can be formulated for use in a food product.

In various embodiments of any aspects described herein, the composition can maintain stable (e.g., no brittle gel formation; or no precipitate formation when the composition is exposed to shear) for at least about 1 month or longer at room temperature or higher. In some embodiments, the composition can maintain stable (e.g., no brittle gel formation; or no precipitate formation when the composition is exposed to shear) for at least about 1 year or longer at room temperature or higher.

The silk fibroin-based compositions and the emulsion compositions described herein can be formulated to any form desirable for a specific application. For example, the composition can be formulated to form a gel or hydrogel, a paste, a lotion, a cream, an ointment, an oil, a liquid, a serum, a shampoo, a foam or mousse, a spray, an aerosol, a stick, a balm, a bar, a scrub, or any combinations thereof.

In some embodiments, the silk fibroin-based compositions and the emulsion compositions can be translucent. In some embodiments, the silk fibroin-based compositions and the emulsion compositions can be flowable. In some embodiments, the silk fibroin-based compositions and the emulsion compositions do not precipitate when exposed to shear.

Methods for producing one or more embodiments of a silk fibroin-based composition and/or an emulsion composition are also provided herein. Accordingly, in one aspect, compositions produced by the methods described herein are provided. In one embodiment, the composition is formulated for use in a personal care product (e.g., a cosmetic, skincare, body care, or hair product). In another embodiment, the composition is formulated for use in a food product.

The method of producing a silk fibroin-based composition and/or an emulsion composition described herein comprises mixing a non-hydrolyzed silk fibroin solution and a humectant agent in a volume ratio of about 1:2 to about 1:100. In some embodiments, the volume ratio of the non-hydrolyzed silk fibroin solution to the humectant agent can be about 1:2 to about 1:10. The humectant agent can be powder, a liquid, a solution, or a suspension. Where the humectant agent is powder, the non-hydrolyzed silk fibroin solution and the humectant agent can be mixed in a weight or mass ratio of about 1:1.1 to about 1:1000, about 1:5 to about 1:750, or about 1:10 to about 1:500.

The rate and/or degree of gelation can be controlled by varying the volume or weight/mass ratio of a non-hydrolyzed silk fibroin solution to a humectant agent. By way of example only, when the volume ratio of the non-hydrolyzed silk fibroin solution to glycerin (an example of a humectant agent) is about 1:4, a gel can be formed within seconds, whereas a ratio of 1:8 can form a gel within about 15 minutes. In addition, the 1:4 silk:glycerin formulations can have a more viscous texture, as compared to 1:8 silk:glycerin formulations.

Any desirable concentration of the non-hydrolyzed silk fibroin solution can be used to produce a composition of various aspects described herein. In some embodiments, the non-hydrolyzed silk fibroin solution can have a concentration of about 1% (w/v or w/w) to about 30% (w/v or w/w). In some embodiments, the non-hydrolyzed silk fibroin solution can have a concentration of about 1% (w/v or w/w) to about 10% (w/v or w/w).

In some embodiments where the non-hydrolyzed silk fibroin solution has a concentration of about 7% (w/v), it can be desirable to maintain the volume ratio of the non-hydrolyzed silk fibroin solution to glycerin at about 1:4 to about 1:8. The inventors have discovered that silk fibroin maintains a random coil configuration at these ratios. If the amount of glycerin is too high relative to the non-hydrolyzed silk fibroin solution (e.g., a silk: glycerin ratio of about 1:10), the mixture of the non-hydrolyzed silk fibroin solution and glycerin does not appear to form a gel. However, if the amount of glycerin is too low relative to the non-hydrolyzed silk fibroin solution (e.g., a silk: glycerin ratio of about 1:2), the relatively-high concentration of the non-hydrolyzed silk fibroin solution can allow formation of beta sheet structures in small regions within the solution, resulting in precipitation of the protein, especially when exposed to shear forces (such as those produced by rubbing the composition on a skin surface).

In some embodiments, the method can further comprise mixing at least one or more additive with the humectant agent and the non-hydrolyzed silk fibroin solution. In some embodiments, the aqueous soluble additive can be mixed with the humectant agent prior to mixing with the non-hydrolyzed silk fibroin solution. In some embodiments, the additive mixed with the humectant agent and/or the non-hydrolyzed silk fibroin solution can be aqueous-soluble.

In some embodiments, the method can further comprise mixing the mixture comprising the non-hydrolyzed silk fibroin solution and the humectant agent with an oil-based phase described herein, for example, to form an emulsion. In some embodiments, the oil-based phase can further comprise a carrier oil and optionally any art-recognized emulsifier. Any carrier oils known in the art, e.g., for use in formulating personal care products, skin care products, body care products, hair products, and/or aromatherapy products, can be used herein. Examples of carriers oil include, without limitations, a synthetic or natural oil derived from seeds, nuts, fruits, flowers, plant-based materials, or any combinations thereof. An appropriate ratio of the carrier oil to the emulsifier can be readily determined by a skilled artisan for different emulsion compositions forms. By way of example only, the volume ratio of the carrier oil to the emulsifier can range from about 2:1 to about 100:1. In some embodiments, the oil-based phase can further comprise an additive described herein.

Yet another aspect provides a method using any compositions of various aspects described herein. The method comprises applying on a target surface one or more embodiments of silk fibroin-based compositions and/or emulsion compositions described herein. The compositions described herein can be applied to a target surface by any means known in the art, e.g., rubbing, spreading, gliding, painting, spraying, extruding, massaging, brushing, smoothing, rolling, or any combinations thereof, depending on the format of the compositions described herein.

By way of example only, when the compositions described herein is flowable or spreadable (e.g., in a form of a serum, a lotion, or a cream), the compositions can be applied on a target surface, e.g., by rubbing, spreading, massaging, smoothing, or any combinations thereof. In other embodiments where the composition and/or emulsion compositions is a solid, e.g., balm, or a stick, the compositions and/or emulsion compositions described herein can be applied by rubbing, rolling, gliding, or any combinations thereof.

Any target surface can be amenable to the application of the compositions described herein. In some embodiments, the target surface can be skin or hair of a subject. In some embodiments, the target surface can be a wound. In some embodiments, the target surface can be a surface of food, for example, where the composition can be used as an edible food-decorating material, e.g., in a form of ink, gel, or paste.

In some embodiments, the method can further comprise maintaining the composition at room temperature for at least about 1 month or longer, wherein the composition remains stable thereafter.

### BRIEF DESCRIPTION OF THE DRAWINGS

FIGS. 1A-1C are photographs showing that a non-hydrolyzed silk fibroin protein solution generally have a short shelf life before spontaneous gelation and thickens when exposed to shear perturbation. FIG. 1A is a photograph showing a (i) freshly-prepared solution of non-hydrolyzed silk fibroin protein that has been degummed (by boiling) for about 30 mins and (ii) a gel formed after the solution is stored for about 1-2 months at 4° C. The spontaneously gelled silk fibroin is considered to be “expired.” FIG. 1B is a photograph showing that expired silk is brittle and exhibits poor aesthetics and spreading quality and therefore is not desirable for use in skin and hair care products. FIG. 1C is a photograph showing that when rubbed on the skin, non-hydrolyzed silk fibroin solution (liquid silk) shear-thickens and forms a “pilled” precipitate and therefore also not desirable for use in skin and hair care products.

FIGS. 2A-2C are experimental data showing distinct properties of ALLSilk™ gel (a composition comprising non-hydrolyzed silk fibroin combined with a humectant, e.g., glycerol). The ALLSilk™ technology is mechanistically different from traditional silk gelation. FIG. 2A shows the amide I and amide II FTIR spectrum of a non-hydrolyzed silk fibroin protein solution, expired silk, glycerin, and the ALLSilk™ flowable gel. The 4° C. gelled silk (also termed as “expired silk” herein, which refers to non-hydrolyzed silk fibroin that has been left for about 1-3 months at about 4° C. until it spontaneously gels) had a prominent peak at 1430 cm⁻¹ indicating high β-sheet content. This peak was absent in the insoluble silk film (which is non-hydrolyzed silk fibroin that has been dried and post-treated, e.g., by water vapor annealing and/or solvent immersion, to induce beta sheet formation), which generally have had minimal β-sheet content. The glycerin had no peak in either the amide I or amide II region. In contrast, the ALLSilk™ technology had a small peak in amide I region and no peak in the amide II region. Regions of interest are demarcated by a dashed line: i=random coil, ii=β-sheet, iii=tyrosine side-chains. FIG. 2B is a photograph showing that gelation of non-hydrolyzed silk fibroin protein was induced by lowering the pH. When 6% non-hydrolyzed silk fibroin protein solution was combined with water at different pHs in a 1:4 volume ratio, gelation was only observed in the pH 1 condition. In contrast, FIG. 2C shows that the ALLSilk™ technology gelled at a wide range of pHs where the level of pH affects the time to gelation. For example, the gelation time of the ALLSilk™ composition is longer at higher pH values.

FIGS. 3A-3H are photographs showing that the ALLSilk™ technology plays a pivotal role in forming emulsions between oil and aqueous phases. If the ALLSilk™ composition is not used in the formulation, the oil and aqueous phases separate and do not form an aesthetically pleasing gel on the skin. By varying the glycerin content of the emulsion, the viscosity of the ALLSilk™ composition can be altered. FIGS. 3A-3B are photographs showing material properties of a composition comprising 8:1 volume ratio of glycerin to non-hydrolyzed silk fibroin in the aqueous phase. FIGS. 3C-3D are photographs showing material properties of a composition comprising 4:1 volume ratio of glycerin to non-hydrolyzed silk fibroin in the aqueous phase. FIGS. 3E-3F are photographs showing material properties of a composition comprising 2:1 volume ration of glycerin to non-hydrolyzed silk fibroin in the aqueous phase. FIGS. 3G-3H are photographs showing material properties of a composition with non-hydrolyzed silk fibroin replaced with water or without the use of an emulsifier.

FIGS. 4A-4C are experimental data showing distinct material properties between hydrolyzed silk (that is commonly incorporated into existing skin and hair care products) and non-hydrolyzed silk (that used in the compositions described herein). FIG. 4A is a SDS-PAGE image of non-hydrolyzed silk fibroin protein (derived from 10-, 30-, and 60-minutes of boiling) and hydrolyzed silk purchased from an outside supplier. The hydrolyzed silk cannot be visualized or detected on the SDS-PAGE because its molecular weight is less than 30 kDa. FIG. 4B shows that non-hydrolyzed silk fibroin protein and hydrolyzed silk protein have distinct fluorescent emission spectra, indicating their secondary and tertiary structure are different. FIG. 4C shows that when non-hydrolyzed silk protein in the compositions described herein is replaced with hydrolyzed silk, no gelling was observed (i=hydrolyzed silk, ii=non-hydrolyzed silk).

FIGS. 5A-5B are SEM images showing hair from the same person treated with or without the ALLSilk™ serum (an exemplary formulation comprising non-hydrolyzed silk fibroin and glycerol in the aqueous phase is shown in Table 2) for about 80 days. Half the hair was left untreated as a control. FIG. 5A shows that over the course of the 80 days, the cuticle of the hair treated with the ALLSilk™ serum remained smooth, while the untreated hair became increasingly rough. FIG. 5B shows that the hair treated with the ALLSilk™ serum was more resilient to bending compared to the control.

### DETAILED DESCRIPTION OF THE INVENTION

Embodiments of various aspects provided herein generally relate to compositions comprising non-hydrolyzed silk fibroin and a humectant, as well as methods of making and using the same. Currently, there are several products on the market that claim to have silk fibroin in the formulation. Some of these products include BioSilk Silk Therapy and Rock Hard Gelee (hydrolyzed silk), CHI Silk Infusion (Ethyl Ester of Hydrolyzed Silk), FX Special Effects Silk Drops Hair Serum (silk protein), Smiss Silk Cream (silk powder, silk amino acids), PRAI Silk Cream Moisturizer (silk powder, silk amino acids) and Silk Serum Hydrating Complex (hydrolyzed silk), Red Flower Plum Blossom Silk Cream (hydrolyzed silk), Natura Bisse Hand Silk Cream (sericin and fibroin silk amino acids), Avon Skin So Soft Signature Silk Body Moisturizing Serum (hydrolyzed silk), Crafters Choice Satin & Silk Lotion (hydrolyzed silk), St. Ives Whipped Silk (hydrolyzed silk protein). However, these products do not use silk fibroin in a non-hydrolyzed form, but in a hydrolyzed form instead, e.g., silk fibroin protein that has been hydrolyzed or broken down into a mixture of small fragments, e.g., less than 30 kDa, less than 20 kDa or lower) and/or constituent amino acids.

In contrast, embodiments of various compositions described herein comprise silk fibroin in a non-hydrolyzed form. Silk fibroin in a non-hydrolyzed or intact protein form has many desirable qualities, e.g., but not limited to, strength, luster, smooth feel, and its ability to absorb moisture that can be beneficial, e.g., to topical hair and skin care. However, once the silk fibroin protein is hydrolyzed, these desirable qualities are lost because they are inherent to the tertiary and quaternary structure of the non-hydrolyzed silk fibroin molecules.

While hydrolyzed silk amino acids exist as an aqueous solution that is relatively easy to work into skin and hair care formulation, non-hydrolyzed or intact silk fibroin solution needs to be prepared in a highly-regulated manner in order to preserve the tertiary and quaternary structure of the protein. Further, a non-hydrolyzed silk fibroin solution can become a brittle gel after 1-2 months of storage at about 4° C., and it can shear-thicken and form precipitates when rubbed on the skin. To overcome these challenges, the inventors have discovered inter alia a novel composition and method for stabilizing non-hydrolyzed silk fibroin protein so that it can be incorporated into beauty products that can provide long-lasting moisture and protection for skin and hair as well as a pleasant aesthetic. In one embodiment, the inventors discovered that the combination of a non-hydrolyzed silk fibroin solution and a humectant (e.g., a glycerin) can produce a composition with a gel-like consistency that is translucent and flowable, which is distinctly different from the brittle gel formed from the silk fibroin solution alone. In particular, when the volume ratio of glycerin to non-hydrolyzed silk fibroin solution is greater than 1:1 (e.g., about 2:1 to about 8:1), the interaction of glycerin and silk fibroin surprisingly provides a gel composition that is stable at room temperature for an extended period of time, e.g., at least about 12 months and does not form precipitation of the protein when sheared on the skin. Accordingly, provided herein are novel compositions and methods of stabilizing non-hydrolyzed silk fibroin proteins, which can be versatile in various applications, e.g., skin and hair products.

### Silk Fibroin-Based Compositions and Emulsion Compositions

One aspect of the inventions provides a silk fibroin-based composition comprising non-hydrolyzed silk fibroin and a humectant agent. In some embodiments, the non-hydrolyzed silk fibroin can have an average molecular weight of at least about 20 kDa or higher, e.g., at least about 30 kDa, at least about 40 kDa, at least about 50 kDa, at least about 100 kDa, at least about 150 kDa, at least about 200 kDa, at least about 250 kDa, at least about 300 kDa, at least about 350 kDa, at least about 400 kDa, at least about 450 kDa or higher. In some embodiments, at least a portion (e.g., at least about 10%) of the non-hydrolyzed silk can have a molecular weight of at least about 50 kDa or higher.

In some embodiments, at least a portion (e.g., at least about 10%, at least about 20%, at least about 30%, at least about 40%, at least about 50%, at least about 60%, at least about 70%, at least about 80%, at least about 90%, at least about 95% or more) of the non-hydrolyzed silk fibroin can maintain its tertiary and/or quaternary structures. As used herein, the terms “tertiary structure” and “quaternary structure” generally have similar meanings understood by those of skill in the art. Tertiary structure refers to the three-dimensional structure of a single polypeptide chain. Quaternary structure refers to the three dimensional structure of a polypeptide having multiple polypeptide chains. Additional information about non-hydrolyzed silk fibroin and its differences from the hydrolyzed silk are further described in the section “Non-hydrolyzed silk fibroin vs. hydrolyzed silk fibroin” below.

Any agent, molecule, reagent, or substance that can absorb moisture from the surrounding, reduce moisture loss from non-hydrolyzed silk fibroin or the composition described herein, and/or promote non-hydrolyzed silk fibroin or the composition described herein to retain moisture, can be selected for use as a humectant agent in the compositions described herein. Examples of the humectant agents include, but are not limited to, phospholipid (e.g., lecithin, panthenol (pro-vitamin B5), glycerin (glycerol)), aloe vera, sorbitol, urea, alpha-hydroxy acids, sodium pyroglutamate, N-acetyl-ethanolamine, sodium lactate, isopropanol, polyalkylene glycols (e.g., ethylene glycol, propylene glycol, hexylene glycol, butylene glycol, dipropylene glycol, triethylene glycol), 1,3-propanediol, polyethylene glycols, synthetic alcohols (e.g., glyceryl coconate, hydroxystearate, myristate, oleate), and any combinations thereof.

Amounts of humectant agents present in the composition of any aspects described herein can be optimized to yield a composition that (1) is shelf-stable, e.g., for at least about 24 hours or longer at about room temperature or higher; and/or (2) does not shear-thicken or form precipitates when sheared. For example, in some embodiments of this aspect and other aspects described herein, the humectant agent can be present in an amount of about 0.1% (w/v or w/w) to about 99.9% (w/v or w/w). In some embodiments, the humectant agent can be present in an amount of at least about 50% (w/v or w/w), at least about 60% (w/v or w/w), at least about 70% (w/v or w/w), at least about 80% (w/v or w/w), at least about 90% (w/v or w/w) or higher (excluding 100%). In some embodiments, the humectant agent can be present in an amount of about 50% (w/v or w/w) to about 99% (w/v or w/w), about 60% (w/v or w/w) to about 99% w/v or w/w), about 70% (w/v or w/w) to about 99% (w/v or w/w), about 80% (w/v or w/w) to about 99% (w/v or w/w), or about 90% (w/v or w/w) to about 99% (w/v or w/w).

In some embodiments, the humectant agent used in the composition described herein can comprise phospholipid (e.g., but not limited to, lecithin, panthenol (pro-vitamin B5), glycerin), aloe vera, sorbital, alpha-hydroxy acids, or any combinations thereof. In one embodiment of this aspect and other aspects described herein, the humectant agent comprises glycerin. While glycerin (also known as glycerol) has been previously used in a silk fibroin composition (e.g., described in the International Patent Application No. WO 2010/042798, the content of which is incorporated herein by reference), it was mainly incorporated as a plasticizer, at a glycerin: silk weight ratio up to 1:1, to make silk materials (e.g., a silk film) more flexible. The '798 patent application does not describe a composition comprising glycerin and silk fibroin in a weight ratio greater than 1:1, or a composition comprising silk fibroin and glycerin in an amount greater than 50% (w/w) as described herein. The inventors have discovered inter alia that the gels formed from a mixture of glycerin and silk fibroin in a ratio greater than 1:1 (e.g., glycerin: silk ration=˜4:1) differ from the silk fibroin-based materials described in the '798 patent application (i.e., a silk fibroin-based material formed from the same components but with a ratio of glycerin to silk fibroin no more than 1:1). For example, unlike flexible silk fibroin films described in the '798 patent applications, the compositions described herein have a gel-consistency with silk fibroin maintained in dominant random-coil content, which likely contributes to its flowability. Accordingly, in some embodiments of this aspect and other aspects described herein, glycerin as a humectant agent can be present in an amount of at least about 50% (w/v or w/w), at least about 60% (w/v or w/w), at least about 70% (w/v or w/w), at least about 80% (w/v or w/w), at least about 90% (w/v or w/w) or higher (excluding 100%). In some embodiments, glycerin can be present in an amount of about 50% (w/v or w/w) to about 99% (w/v or w/w), about 60% (w/v or w/w) to about 99% w/v or w/w), about 70% (w/v or w/w) to about 99% (w/v or w/w), about 80% (w/v or w/w) to about 99% (w/v or w/w), or about 90% (w/v or w/w) to about 99% (w/v or w/w).

In some embodiments of this aspect and other aspects described herein, the non-hydrolyzed silk fibroin and the humectant agent can be in any ratio, provided that the amount of the humectant agent is more than that of the non-hydrolyzed silk fibroin. For example, the non-hydrolyzed silk fibroin and the humectant agent can be present in a volume ratio of about 1:1.1 to about 1:100, or about 1:2 to about 1:100, or about 1:2 to about 1:10, about 1:2 to about 1:8, or about 1:4 to about 1:8. Stated another way, the non-hydrolyzed silk fibroin and the humectant agent can be present in a weight/mass ratio of about 1:1.1 to 1:1000, about 1:5 to about 1:750, or about 1:10 to about 1:500. Depending on the selection of the humectant agent, and/or the non-hydrolyzed silk fibroin concentration, the ratio of the non-hydrolyzed silk fibroin to the humectant agent can be optimized accordingly, e.g., by examining the material property and/or rheology of the composition comprising different ratios of the non-hydrolyzed silk fibroin to the humectant agent using the methods as shown in Example 1 or any methods known to a skilled artisan.

In some embodiments of this aspect and other aspects described herein, the volume or weight ratio of the non-hydrolyzed silk fibroin to the humectant agent can be adjusted to yield a translucent composition. Accordingly, in some embodiments, the composition described herein and hereafter can be translucent. The term “translucent” as used herein generally refers to a characteristic whereby light passes through an object. The light may diffuse through the object and may be scattered when transmitted through the substrate. The term “translucent”, as used herein can also include “frosted”, “glittered”, “pearlescence,” “semi-transparent” and the like. In some embodiments, the term “translucent” can refer to a composition having a turbidity of 1 to 90 ppm, e.g., determined according to determination with turbidimeter with integrating sphere, on a pure kaolin basis (provided by JIS-K1010, 8). In general, a transparent composition has a turbidity of 1 ppm or below while a clouded emulsion such as a milky composition has a turbidity of 90 ppm or above. Thus, a translucent composition can have a turbidity between 1 ppm and 90 ppm. In other embodiments, translucency of a composition can be quantified using a “contrast ratio method” for measuring opacity. The opacity value gives an inverse measurement of translucency. According to this method, a colorimeter (e.g., Pacific Scientific Colorgard System/05 Colorimeter, similar to a Hunter Tristimulus Colorimeter) can be calibrated and used to obtain two measurements. The first measurement is taken with a translucent composition backed by a white tile or white background, placed over a light source, to achieve maximum reflectance of the light which passes through the sample. The second measurement is then taken with the translucent bar backed by a black tile or a black background, placed over a light source, for minimum reflectance of light through the sample. The percent opacity is then defined as the value of the second measurement divided by the first measurement multiplied by one hundred. A translucent composition is not as clear as a transparent composition, i.e., they possess a higher degree of opacity than that of a transparent composition. A composition is deemed to be translucent when its percent opacity is from about 40 to about 85%.

In some embodiments of this aspect and other aspects described herein, the volume or weight ratio of the non-hydrolyzed silk fibroin to the humectant can be adjusted to yield a flowable or spreadable composition. For example, in one embodiment where the humectant agent comprises glycerin, the 4:1 glycerin:silk composition appears to have a more viscous texture compared to the 8:1 glycerin:silk composition. Accordingly, in some embodiments, the composition described herein and hereafter can be flowable or readily spreadable on a surface. The term “flowable” in this context applies to compositions whose consistencies range from those which can be described as shape-sustaining but readily deformable, e.g., those which behave like putty, to those which are runny. Example forms of flowable compositions include, but not limited to, pastes, creams, lotions, and/or gels. In some embodiments, the flowable composition is capable of passing through a syringe and cannula. As used herein, the term “spreadable” means that the composition can be spread over a surface or an area.

In some embodiments of this aspect and other aspects described herein, the volume or weight ratio of the non-hydrolyzed silk fibroin to the humectant can be adjusted to yield a composition that does not shear-thicken or precipitate when exposed to shear. Accordingly, in some embodiments, the composition described herein and hereafter can be resistant to shear-thickening or does not readily form precipitates or particulates upon exposure to shear, e.g., for at least about 15 seconds, at least about 30 seconds, at least about 1 minute, at least about 5 minutes or more. The term “shear-thickening” generally refers to a composition having a lower consistency at zero or low shear stress than in presence of shear stress or higher shear stress values. In some embodiments, the composition described herein can be shear-thinning upon exposure to shear, e.g., for at least about 15 seconds, at least about 30 seconds, at least about 1 minute, at least about 5 minutes or more. As used herein, the term “shear-thinning” refers to phenomena of decreasing viscosity with increasing shear rate. In some embodiments, the composition described herein can substantially maintain their viscosity or rheology upon exposure to shear, e.g., for at least about 15 seconds, at least about 30 seconds, at least about 1 minute, at least about 5 minutes or more.

The compositions described herein are distinct from a composition comprising non-hydrolyzed silk fibroin without a humectant agent. For example, the inventors have discovered that when non-hydrolyzed silk fibroin protein forms a gel in the absence of a humectant, the resulting gel contains a prominent beta-sheet content, as opposed to more random coil conformation maintained in the silk fibroin-based gels comprising a humectant.

Thus, in some embodiments, the composition described herein can have a larger amount of silk fibroin in random coil conformation, as compared to a composition comprising the non-hydrolyzed silk fibroin in the absence of the humectant agent. For example, the compositions described herein can have silk fibroin in random coil conformation in an amount that is at least about 10% or more, e.g., at least about 20%, at least about 30%, at least about 40%, at least about 50%, at least about 60%, at least about 70%, at least about 80%, at least about 90%, at least about 95% or more, larger than when the humectant agent is absent. In some embodiments, the composition described herein can have a random-coil silk fibroin content of at least about 10%, at least about 20%, at least about 30%, at least about 40%, at least about 50%, at least about 60%, at least about 70%, at least about 80%, at least about 90%, at least about 95% or more.

In some embodiments, the composition described herein can have a smaller amount of silk fibroin in beta-sheet conformation, as compared to a composition comprising the non-hydrolyzed silk fibroin in the absence of the humectant agent. For example, the composition described herein can have silk fibroin in beta sheet conformation in an amount that is at least about 10% or more, e.g., at least about 20%, at least about 30%, at least about 40%, at least about 50%, at least about 60%, at least about 70%, at least about 80%, at least about 90%, at least about 95% or more, smaller than when the humectant agent is absent. In some embodiments, the composition described herein can have a beta-sheet silk fibroin content of no more than 60%, no more than 50%, no more than 40%, no more than 30%, no more than 20%, no more than 10%, no more than 5%, no more than 3% or less.

In some embodiments, the composition described herein can have silk fibroin in random-coil conformation and beta-sheet conformation at a ratio (e.g., mass ratio) of about 2:1 to about 100:1, or about 5:1 to about 75:1, or about 20:1 to about 50:1.

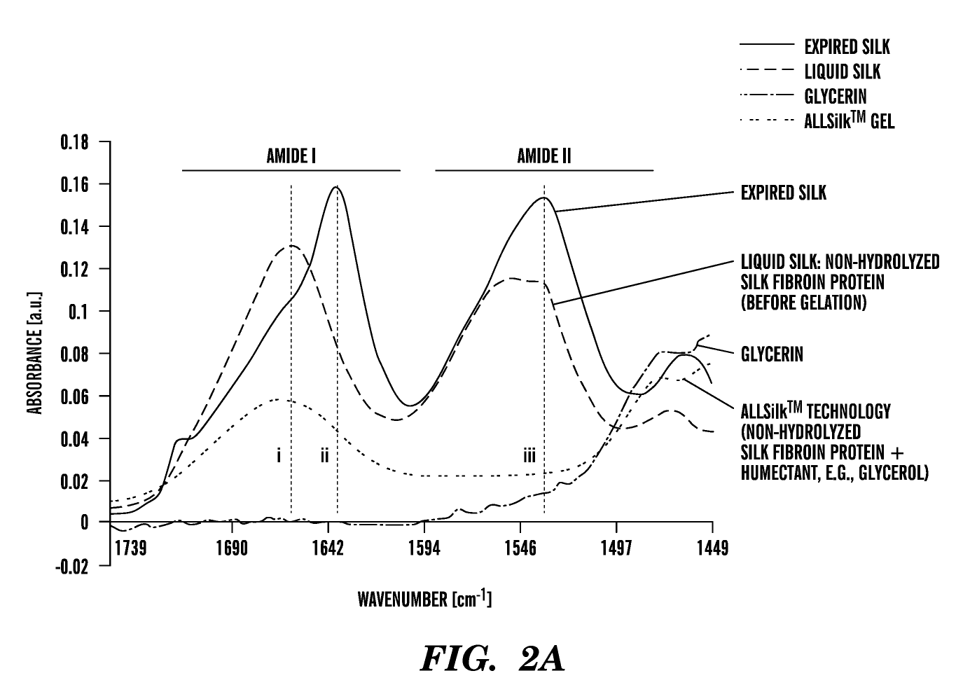

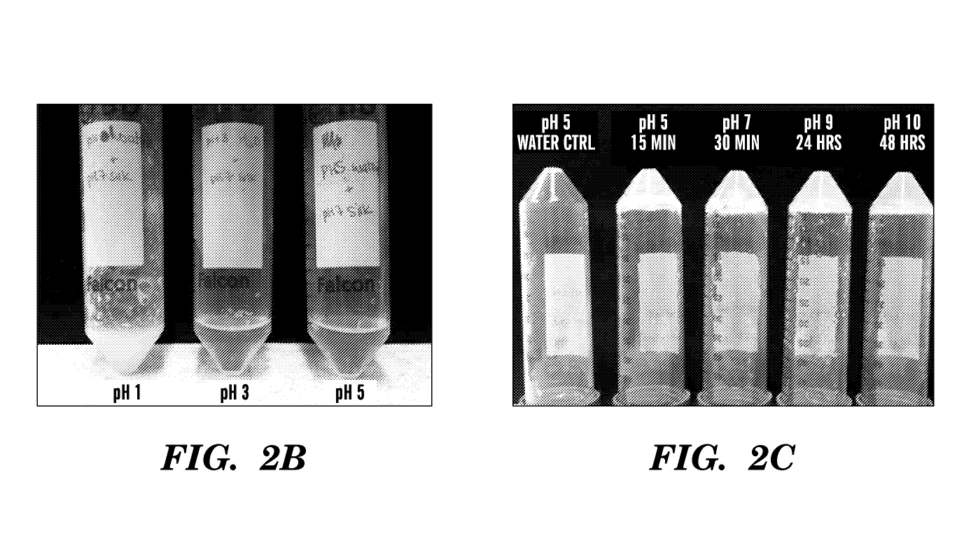

In some embodiments, the composition described herein does not have a peak in the amide II region (e.g., between ˜1475 cm⁻¹ and ˜1560 cm⁻¹), as determined in a Fourier Transform Infrared Spectroscopy (FTIR) spectrum, e.g., as shown in FIG. 2A.

It has been previously reported that silk gelation can be controllably induced by reducing pH of the silk fibroin solution (see, e.g., U.S. Patent Application No. US 2011/0171239, the content of which is incorporated herein by reference, for pH-induced silk gelation). In contrast to the traditional silk gelation, in some embodiments, the composition described herein can form a gel regardless of pH values ranging from pH˜1 to pH˜14. However, the pH of the composition described herein can affect the kinetics of silk fibroin gelation, e.g., time to gelation from seconds to hours. For example, as shown in FIG. 2C, a composition comprising a 6% non-hydrolyzed silk fibroin solution and glycerin at a ratio of about 1:4 can gel from minutes to hours, depending on the pH of the composition. Without wishing to be bound by theory, decreasing the pH of the composition can generally decrease the time to gelation.

In some embodiments, the composition comprising non-hydrolyzed silk fibroin and a humectant agent can be prepared in an aqueous phase, which can then be mixed with an oil-based phase or a wax-based phase to form an emulsion. The inventors have discovered that when an aqueous mixture of a humectant (e.g., glycerin) and non-hydrolyzed silk fibroin was added to an oil-based solution, the non-hydrolyzed silk fibroin plays a role in emulsifying the oil and aqueous phases. For example, addition of soy lecithin alone is not a sufficient emulsifier to emulsify the aqueous and oil phases. Thus, the soy lecithin alone is not sufficient to stabilize an emulsion (e.g., an oil-in-water emulsion), where it contains hydrolyzed silk instead of non-hydrolyzed silk fibroin. The use of hydrolyzed silk fibroin or water in place of non-hydrolyzed silk fibroin resulted in phase separation, even in the presence of soy lecithin. Only when non-hydrolyzed silk fibroin was mixed with the aqueous and oil phases did the ingredients form a single-phase gel. Thus, in some embodiments, non-hydrolyzed silk fibroin can act as an emulsifying agent in the compositions described herein and/or aid in an emulsification process, allowing aqueous and oil phases to mix and form a single-phase gel (e.g., a flowable single-phase gel). This has not been previously reported for non-hydrolyzed silk fibroin, and, without wishing to be bound by theory, the ability of non-hydrolyzed silk fibroin to act as an emulsifying agent can be in part due to the protein having both hydrophilic and hydrophobic regions.

Accordingly, in another aspect, an emulsion composition comprising non-hydrolyzed silk fibroin is also provided herein. The emulsion composition comprises an aqueous-based phase, and an oil-based phase or a wax-based phase, wherein the aqueous-based phase comprises non-hydrolyzed silk fibroin and a humectant agent described herein. As used herein, the term “emulsion” refers to a mixture of two immiscible substances. One of the substances which is referred to as the dispersed phase is dispersed in the other substance referred to as the continuous phase. Emulsions are generally unstable mixtures and do not form spontaneously, thus, in order to mix the continuous and dispersed phases and form the emulsion, an energy input is required. This energy can be applied by any of shaking, stirring, homogenizing, spray processing, high pressure pumping and ultrasonic emulsification for example.

Over time however, the emulsions formed tend to revert to the stable state of separate oil and aqueous layers. Accordingly, in some embodiments, the emulsion composition can further comprise any natural or synthetic emulsifier known in the art, including, for example, liquid soy lecithin, solid soy lecithin, honey, beeswax, cetyl alcohol, or any combinations thereof. The addition of an emulsifier can increase the kinetic stability of emulsions so that, once formed, the emulsion does not change significantly in long term storage.

In some embodiments, the oil-based phase can comprise a carrier oil. For example, a carrier oil can be a synthetic or natural oil derived from seeds, nuts, fruits, flowers, plant-based materials, or any combinations thereof. Examples of carrier oils include, but are not limited to, sweet almond oil, olive oil, wheat germ oil, castor oil, avocado oil, rosehip seed oil, calendula oil, jojoba oil, grape seed oil, apricot kernel oil, flaxseed oil, hazelnut oil, walnut oil, pecan nut oil, macadamia nut oil, sesame oil, emu oil, coconut oil, and vegetable oils such as for example safflower oil, sunflower oil, canola oil, soybean oil, and peanut oil, and any combinations thereof.

In some embodiments, the wax-based phase can comprise, e.g., but not limited to, butters, wax, paraffin wax, paraffin oil, petrolatum, or any combinations thereof.

While the humectant agent can be present in any appropriate amount in the aqueous-based phase, in some embodiments, the humectant agent can be present in an amount of at least about 50% (w/v or w/w), at least about 60% (w/v or w/w), at least about 70% (w/v or w/w), at least about 80% (w/v or w/w), at least about 90% (w/v or w/w) or higher (excluding 100%) of the aqueous-based phase. In some embodiments, the humectant agent can be present in an amount of about 50% (w/v or w/w) to about 99% (w/v or w/w), about 60% (w/v or w/w) to about 99% (w/v or w/w), about 70% (w/v or w/w) to about 99% (w/v or w/w), about 80% (w/v or w/w) to about 99% (w/v or w/w), or about 90% (w/v or w/w) to about 99% (w/v or w/w), of the aqueous-based phase.

In some embodiments where glycerin is a selected humectant agent, glycerin can be present in an amount of at least about 50% (w/v or w/w), at least about 60% (w/v or w/w), at least about 70% (w/v or w/w), at least about 80% (w/v or w/w), at least about 90% (w/v or w/w) or higher (excluding 100%) of the aqueous-based phase. In some embodiments, glycerin can be present in an amount of about 50% (w/v or w/w) to about 99% (w/v or w/w), about 60% (w/v or w/w) to about 99% (w/v or w/w), about 70% (w/v or w/w) to about 99% (w/v or w/w), about 80% (w/v or w/w) to about 99% (w/v or w/w), or about 90% (w/v or w/w) to about 99% (w/v or w/w), of the aqueous-based phase.

In some embodiments, the non-hydrolyzed silk fibroin can be present in an amount of about 1% (w/v or w/w) to about 30% (w/v or w/w), or about 1% (w/v or w/w) to about 10% (w/v or w/w) of the aqueous-based phase. In one embodiment, the non-hydrolyzed silk fibroin can be present in an amount of about 5% (w/v or w/w) to about 8% (w/v or w/w).

In some embodiments, the non-hydrolyzed silk fibroin and the humectant agent in the aqueous-based phase can be present in any ratio, provided that the amount of the humectant agent is more than that of the non-hydrolyzed silk fibroin. For example, in the aqueous-based phase, the non-hydrolyzed silk fibroin and the humectant agent can be present in a volume ratio of about 1:1.1 to about 1:100, or about 1:2 to about 1:100, or about 1:2 to about 1:10, about 1:2 to about 1:8, or about 1:4 to about 1:8. Stated another way, the non-hydrolyzed silk fibroin and the humectant agent can be present in a weight/mass ratio of about 1:1.1 to 1:1000, about 1:5 to about 1:750, about 1:10 to about 1:500, or about 1:30 to about 1:250 in the aqueous-based phase.

The ratio of the aqueous-based phase to the oil-based phase can vary to form different types of emulsion compositions, e.g., water-in-oil compositions or oil-in-water compositions. Accordingly, the ratio of the aqueous-based phase to the oil-based phase can vary from about 1:100 to about 100:1, or from about 1:25 to about 25:1, or from about 1:10 to about 10:1. In some embodiments, the ratio of the aqueous-based phase to the oil-based phase is about 1:1 to about 20:1. In one embodiment, the ratio of the aqueous-based phase to the oil-based phase is about 9:1.

In some embodiments, the emulsion composition can comprise at least one or more additives. For example, at least one additive can be present in the aqueous-based phase. Additionally or alternatively, the oil-based phase can comprise at least one or more additives.

Compositions of various aspects described herein are versatile and can be adapted for various applications, e.g., personal care (e.g., cosmetic, skin care, body care, hair care), therapeutic use (e.g., wound healing), or any applications where a flowable or spreadable composition is desirable. By the term “personal care” is meant any substance or produces applied to a human body for improving appearance, providing protection from surrounding environment, cleansing, odor control and/or general aesthetics. Nonlimiting examples of personal care applications include leave-on skin lotions and creams, shampoos, hair conditioners, hair serum, shower cream or gels, antiperspirants, deodorants, dental products such as toothpaste, shave creams, depilatories, cosmetic products (e.g., lipsticks, foundations, mascara), sunless tanners and sunscreen lotions.

Accordingly, the silk fibroin-based compositions and the emulsion compositions described herein can be formulated to any form desirable for a specific application. For example, the composition can be formulated to form a gel or hydrogel, a paste, a lotion, a cream, an ointment, an oil, a liquid, a serum, a shampoo, a foam or mousse, a spray, an aerosol, a stick, a balm, a bar, a scrub, or any combinations thereof.

In some embodiments, the silk fibroin-based composition and/or the emulsion composition described herein can further comprise an additive, e.g., to suit the need of an application. Examples of an additive include, but are not limited to, aqueous-soluble components, oil-soluble components, wax-soluble components, essential oils, emulsifiers, surfactants, flavors, fragrance, nutraceuticals, vitamins, therapeutic agents, cosmeceuticals, preservatives, botanical extracts or distillates, antioxidants, anti-bacterial agents, anti-inflammatory agents, cosmetically-acceptable agents, skin-lightening agents, skin-evening agents, anti-reddening agents, viscosity modifiers, emollients, nano- or micro-particles, photothermal elements (e.g., gold nanoparticles), wound healing-promoting agents, and any combinations thereof.

One of skill in the art can determine appropriate additives and amounts thereof based on different applications. In one embodiment, the composition can be formulated for use in a personal care product (e.g., a cosmetic, skincare, body care, or hair product). In one embodiment, the composition can be formulated for use in a food product.

In some embodiments, a silk fibroin-based composition or an emulsion composition described herein can further comprise at least one biopolymer, including at least two biopolymers, at least three biopolymers or more. For example, a silk fibroin-based composition or an emulsion composition described herein can comprise one or more biopolymers in a total concentration of about 0.1 wt % to about 50 wt %, about 1 wt % to about 40 wt %, about 5 wt % to about 30 wt %, or about 10 wt % to about 20 wt %. In some embodiments, the biopolymer(s) can be incorporated homogenously or heterogeneously into a silk fibroin-based composition or an emulsion composition described herein. In other embodiments, the biopolymer(s) can be coated on a surface of a silk fibroin-based composition or an emulsion composition described herein. In some embodiments, the biopolymer(s) can be covalently or non-covalently linked to silk fibroin a silk fibroin-based composition or an emulsion composition described herein. In some embodiments, the biopolymer(s) can be blended with non-hydrolyzed silk fibroin within a silk fibroin-based composition or an emulsion composition described herein. Examples of the biopolymer can include biocompatible and/or biodegradable polymer, e.g., but are not limited to, polyethylene oxide (PEO), polyethylene glycol (PEG), collagen, fibronectin, keratin, polyaspartic acid, polylysine, alginate, chitosan, chitin, hyaluronic acid, pectin, polycaprolactone, polylactic acid, polyglycolic acid, polyhydroxyalkanoates, dextrans, polyanhydrides, polymer, PLA-PGA, polyanhydride, polyorthoester, polycaprolactone, polyfumarate, collagen, chitosan, alginate, hyaluronic acid, other biocompatible and/or biodegradable polymers and any combinations thereof. See, e.g., International Application Nos.: WO 04/062697; WO 05/012606. The contents of the international patent applications are all incorporated herein by reference. Depending on various applications (e.g., in a personal care, or wound dressing), in some embodiments, a silk fibroin-based composition or an emulsion composition described herein can include about 1% to about 30%, or about 2% to about 20%, or about 5% to about 10% polyethylene oxide (e.g., PEO with a molecular weight of about 500, 000 to about 1,500,000). In other embodiments, the non-hydrolyzed silk fibroin/PEO blend ratio in a silk fibroin-based composition or an emulsion composition described herein can vary from about 1:100 to about 100:1. In some embodiments, the silk fibroin/PEO blend ratio in a silk fibroin-based composition or an emulsion composition described herein can vary from about 2:1 to about 4:1. See, e.g., International Application No.: WO 2011/008842, the content of which is incorporated herein by reference.

In some embodiments, a silk fibroin-based composition or an emulsion composition described herein can further comprise at least one active agent as described below. The active agent can be dispersed homogeneously or heterogeneously within non-hydrolyzed silk fibroin, or dispersed in a gradient, e.g., using the carbodiimide-mediated modification method described in the U.S. Patent Application No. US 2007/0212730. In some embodiments, the active agent can be coated on a surface of the silk fibroin-based composition or an emulsion composition described herein, e.g., via diazonium coupling reaction (see, e.g., U.S. Patent Application No. US 2009/0232963), and/or avidin-biotin interaction (see, e.g., International Application No.: WO 2011/011347). Non-limiting examples of the active agents can include cells, proteins, peptides, nucleic acids, nucleic acid analogs, nucleotides or oligonucleotides, peptide nucleic acids, aptamers, antibodies or fragments or portions thereof, antigens or epitopes, hormones, hormone antagonists, growth factors or recombinant growth factors and fragments and variants thereof, cell attachment mediators, cytokines, enzymes, antibiotics or antimicrobial compounds, viruses, toxins, therapeutic agents and prodrugs thereof, small molecules, and any combinations thereof. See, e.g., the International Patent Application No. WO/2012/145739 for compositions and methods for stabilization of active agents with silk fibroin. In some embodiments, an active agent can be genetically fused to non-hydrolyzed silk fibroin to form a fusion protein. The contents of the aforementioned patent applications are incorporated herein by reference.

Any amounts of an active agent can be present in the silk fibroin-based compositions or emulsion compositions described herein. For example, in some embodiments, an active agent can be present in a silk fibroin-based composition or an emulsion composition described herein at a concentration of about 0.001 wt % to about 50 wt %, about 0.005 wt % to about 40 wt %, about 0.01 wt % to about 30 wt %, about 0.05 wt % to about 20 wt %, about 0.1 wt % to about 10 wt %, or about 0.5 wt % to about 5 wt %.

In various embodiments of any aspects described herein, the composition can maintain stable (e.g., maintain at least one property of the composition such as translucency, flowability, silk fibroin protein stability, no brittle gel formation; or no precipitate formation when the composition is exposed to shear) over a wide range of temperatures (e.g., any temperature above 0° C., e.g., at room temperature or above, or at body temperature or above) for an extended period of time. In some embodiments, the translucent compositions described herein can remain stably translucent over an extended period of time, without showing any precipitation, separation nor clouding. The phrase “extended period of time” refers to a duration of time of at least about 1 month or longer, including, e.g., at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 18 months, at least about 24 months or longer.

Non-Hydrolyzed Silk Fibroin Vs. Hydrolyzed Silk Fibroin

Silk fibroin is a particularly appealing protein polymer candidate to be used for various embodiments described herein, e.g., because of its versatile processing e.g., all-aqueous processing (Sofia et al., 54 J. Biomed. Mater. Res. 139 (2001); Perry et al., 20 Adv. Mater. 3070-72 (2008)), relatively easy functionalization (Murphy et al., 29 Biomat. 2829-38 (2008)), and biocompatibility (Santin et al., 46 J. Biomed. Mater. Res. 382-9 (1999)). For example, silk has been approved by U.S. Food and Drug Administration as a tissue engineering scaffold in human implants. See Altman et al., 24 Biomaterials: 401 (2003).

As used herein, the term “silk fibroin” includes silkworm fibroin and insect or spider silk protein. See e.g., Lucas et al., 13 Adv. Protein Chem. 107 (1958). Any type of silk fibroin can be used according to different aspects described herein. Silk fibroin produced by silkworms, such as Bombyx mori, is the most common and represents an earth-friendly, renewable resource. For instance, silk fibroin used in a silk fibroin-based composition described herein can be attained by extracting sericin from the cocoons of B. mori. Organic silkworm cocoons are also commercially available. There are many different silks, however, including spider silk (e.g., obtained from Nephila clavipes), transgenic silks, genetically engineered silks, such as silks from bacteria, yeast, mammalian cells, transgenic animals, or transgenic plants (see, e.g., WO 97/08315; U.S. Pat. No. 5,245,012), and variants thereof, that can be used. In some embodiments, silk fibroin can be derived from other sources such as spiders, other silkworms, bees, and bioengineered variants thereof. In some embodiments, silk fibroin can be extracted from a gland of silkworm or transgenic silkworms (see, e.g., WO 2007/098951).

In some embodiments, the silk fibroin can include an amphiphilic peptide. In other embodiments, the silk fibroin can exclude an amphiphilic peptide. “Amphiphilic peptides” possess both hydrophilic and hydrophobic properties. Amphiphilic molecules can generally interact with biological membranes by insertion of the hydrophobic part into the lipid membrane, while exposing the hydrophilic part to the aqueous environment. In some embodiment, the amphiphilic peptide can comprise a RGD motif. An example of an amphiphilic peptide is a 23RGD peptide having an amino acid sequence: HOOC-Gly-ArgGly-Asp-Ile-Pro-Ala-Ser-Ser-Lys-Gly-Gly-Gly-Gly-SerArg-Leu-Leu-Leu-Leu-Leu-Leu-Arg-NH2. Other examples of amphiphilic peptides include the ones disclosed in the U.S. Patent App. No.: US 2011/0008406, the content of which is incorporated herein by reference.

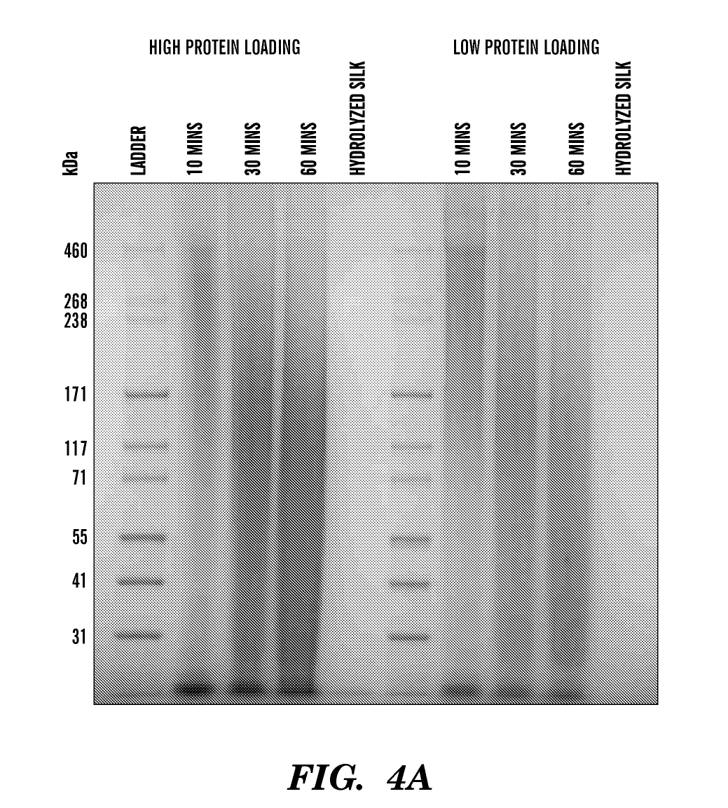

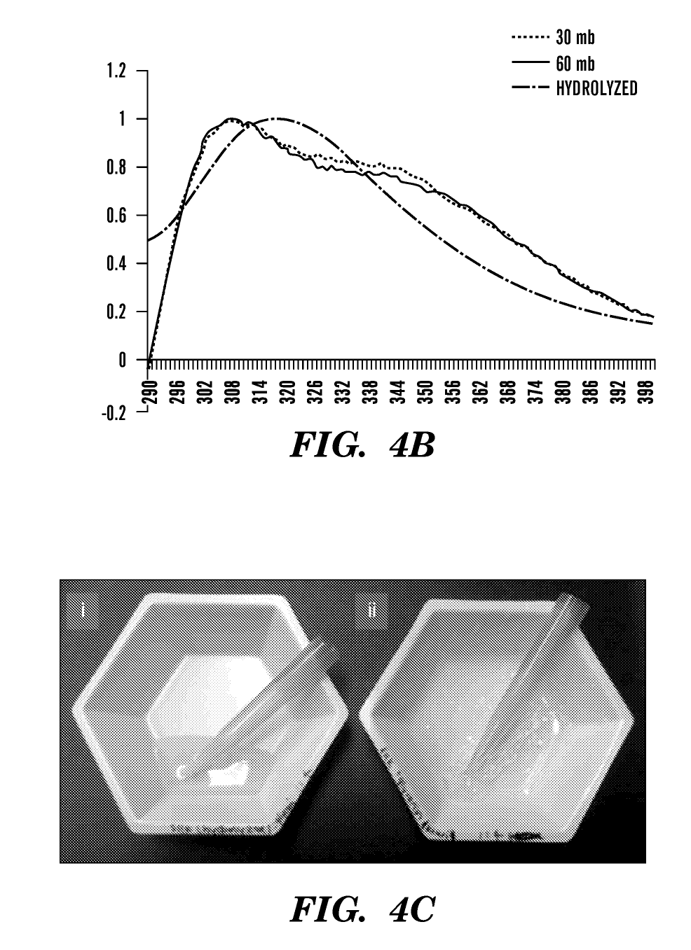

Existing skin and hair care products contain silk fibroin in a hydrolyzed form. Hydrolyzed silk is produced by hydrolyzing or breaking down silk proteins into smaller peptide chains, e.g., less than 30 kDa, less than 20 kDa, or lower, or constituent amino acids. In some embodiments, hydrolyzed silk refers to silk amino acids that are no different from glycine, alanine, serine derived from non-silk sources. Hydrolyzed silk does not form any tertiary and/or quaternary structure and have distinct material properties from non-hydrolyzed silk fibroin. For example, FIG. 4A shows the electrophoretic mobility of the hydrolyzed and non-hydrolyzed silk fibroin protein solutions assessed using SDS-PAGE gels. No protein band from the hydrolyzed silk fibroin sample was detected on the SDS-PAGE gel, indicating that the hydrolyzed silk do not contain any proteins having molecular weights greater than the lowest detectable marker, i.e., ˜30 kDa. In contrast, non-hydrolyzed silk fibroin sample or intact silk fibroin was determined to have a range of molecular weights from less than 30 kDa to at least about 460 kDa (FIG. 4A). The range of molecular weights for non-hydrolyzed silk fibroin can be controlled with boiling time (minutes of boiling—abbreviated as “mb”) during degumming. For example, FIG. 4A indicates that increasing boiling time from about 10 minutes to about 60 minutes forms a mixture of silk fibroin proteins, a portion of which have lower molecular weights as degumming time increases.

Accordingly, the term “hydrolyzed silk” as used herein refers to silk peptide chains or amino acids with a molecular weight of less than 30 kDa, less than 20 kDa, less than 10 kDa, less than 5 kDa, less than 1 kDa or smaller. Stated another way, the hydrolyzed silk is generally free of silk proteins with molecular weights above 30 kDa, above, 40 kDa, above 50 kDa, above 100 kDa or higher.

In contrast, the non-hydrolyzed silk fibroin has an average molecule weight higher than that of the hydrolyzed silk, e.g., by at least about 10%, at least about 20%, at least about 30%, at least about 40%, at least about 50%, at least about 60%, at least about 70%, at least about 80%, at least about 90%. In some embodiments, the non-hydrolyzed silk fibroin has an average molecular weight higher that that of the hydrolyzed silk by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold or higher.

As used herein, the term “average molecular weight” generally means the mean or average of a molecular weight distribution. In some embodiments, the non-hydrolyzed silk fibroin can have an average molecule weight of at least about 20 kDa, at least about 30 kDa, at least about 40 kDa, at least about 50 kDa, at least about 100 kDa, at least about 150 kDa, at least about 200 kDa, at least about 250 kDa, at least about 300 kDa, at least about 350 kDa, at least about 400 kDa, at least about 450 kDa or higher.

In some embodiments, the non-hydrolyzed silk fibroin can comprise at least one or more silk fibroin molecules having a molecular weight of at least about 50 kDa, at least about 60 kDa, at least about 70 kDa, at least about 100 kDa, at least about 150 kDa, at least about 200 kDa, at least about 250 kDa, at least about 300 kDa, at least about 350 kDa, at least about 400 kDa, at least about 450 kDa or higher.

In some embodiments, a portion of the non-hydrolyzed silk fibroin (e.g., at least about 10%, at least about 20%, at least 30%, at least about 40%, at least about 50%, at least about 60%, at least about 70%, at least about 80%, at least about 90% or higher) can have a molecular weight of at least about 50 kDa, at least about 60 kDa, at least about 70 kDa, at least about 100 kDa, at least about 150 kDa, at least about 200 kDa, at least about 250 kDa, at least about 300 kDa, at least about 350 kDa, at least about 400 kDa, at least about 450 kDa or higher.

In addition to the difference in the molecular weights, the hydrolyzed silk solution displays a different fluorescent emission spectrum as compared to those of 30 mb and 60 mb non-hydrolyzed silk fibroin solutions (FIG. 4B), indicating that hydrolyzed silk and non-hydrolyzed silk fibroin proteins have distinct secondary and tertiary structures. Further, when the non-hydrolyzed silk fibroin protein component was replaced with hydrolyzed silk at a ratio of 4:1 glycerol to silk, the resulting hydrolyzed silk-glycerol mixture did not exhibit gelation (FIG. 4C, panel i). In contrast, a gel is formed when non-hydrolyzed silk fibroin was mixed with glycerol at the same volume ratio (FIG. 4C, panel ii).

A non-hydrolyzed silk fibroin solution can be prepared by any conventional method known to one skilled in the art. For example, B. mori cocoons are boiled for varying times (e.g., about 10 minutes to about 60 minutes, depending on the desirable ranges of the molecular weights) in an aqueous solution. In one embodiment, the aqueous solution is about 0.02M Na₂CO₃. The cocoons are rinsed, for example, with water to extract the sericin proteins and the extracted silk is dissolved in an aqueous salt solution. Salts useful for this purpose include lithium bromide, lithium thiocyanate, calcium nitrate or other chemicals capable of solubilizing silk. In some embodiments, the extracted non-hydrolyzed silk fibroin is dissolved in about 8M-12 M LiBr solution. The salt is consequently removed using, for example, dialysis.

If necessary, the non-hydrolyzed silk fibroin solution can then be concentrated using, for example, dialysis against a hygroscopic polymer solution, for example, PEG, a polyethylene oxide, amylose or sericin. In some embodiments, the PEG is of a molecular weight of 8,000-10,000 g/mol and has a concentration of 5 wt %-50 wt % (e.g., about 15 wt %). A slide-a-lyzer dialysis cassette (Pierce, MW CO 3500) can be used. However, any dialysis system can be used. The dialysis can be performed for a time period sufficient to result in a final concentration of aqueous silk solution between about 10 wt %-about 50 wt %. In some embodiments, the dialysis can be performed for a time period sufficient to result in a final concentration of aqueous silk solution at about 30 wt %. In most cases dialysis for 5-20 hours (e.g., 18 14 hours) is sufficient and longer dialysis is also permitted. See, for example, International Application No. WO 2005/012606, the content of which is incorporated herein by reference.

Alternatively, the non-hydrolyzed silk fibroin solution can be produced using organic solvents. Such methods have been described, for example, in Li, M., et al., J. Appl. Poly Sci. 2001, 79, 2192-2199; Min, S., et al. Sen'I Gakkaishi 1997, 54, 85-92; Nazarov, R. et al., Biomacromolecules 2004 May-June; 5(3):718-26. For example, an exemplary organic solvent that can be used to produce a non-hydrolyzed silk fibroin solution includes, but is not limited to, hexafluoroisopropanol.

Non-hydrolyzed silk fibroin can be present in the silk fibroin-based compositions and/or emulsion compositions described herein at any amounts, provided that the amount of the non-hydrolyzed silk fibroin is lower than the amount of the humectant agent within the composition. In some embodiments, non-hydrolyzed silk fibroin can be present in the silk fibroin-based compositions and/or emulsion compositions in an amount of about 1 wt % to about 49.9 wt %, about 3 wt % to about 45 wt %, about 5 wt % to about 40 wt %, or about 10 wt % to about 35 wt %, of the total weight. In some embodiments of this aspect and other aspects described herein, the non-hydrolyzed silk fibroin can be present in an amount of about 1% (w/v or w/w) to about 30% (w/v or w/w), or about 1% (w/v or w/w) to about 10% (w/v or w/w). In some embodiments, the non-hydrolyzed silk fibroin can be present in an amount of about 5% (w/v or w/w) to about 8% (w/v or w/w).

In various embodiments, the non-hydrolyzed silk fibroin can be modified for different applications and/or desired mechanical, rheology, or chemical properties (e.g., to facilitate formation of a gradient of an additive (e.g., an active agent) in silk fibroin-based composition). One of skill in the art can select appropriate methods to modify non-hydrolyzed silk fibroins, e.g., depending on the side groups of the silk fibroins, desired reactivity of the silk fibroin and/or desired charge density on the silk fibroin. In one embodiment, modification of non-hydrolyzed silk fibroin can use the amino acid side chain chemistry, such as chemical modifications through covalent bonding, or modifications through charge-charge interaction. Exemplary chemical modification methods include, but are not limited to, carbodiimide coupling reaction (see, e.g. U.S. Patent Application. No. US 2007/0212730), diazonium coupling reaction (see, e.g., U.S. Patent Application No. US 2009/0232963), avidin-biotin interaction (see, e.g., International Application No.: WO 2011/011347) and pegylation with a chemically active or activated derivatives of the PEG polymer (see, e.g., International Application No. WO 2010/057142). Non-hydrolyzed silk fibroin can also be modified through gene modification to alter functionalities of the silk protein (see, e.g., International Application No. WO 2011/006133). For instance, the non-hydrolyzed silk fibroin can be genetically modified, which can provide for further modification of the silk such as the inclusion of a fusion polypeptide comprising a fibrous protein domain and a mineralization domain, which can be used to form an organic-inorganic composite. See WO 2006/076711. In some embodiments, the non-hydrolyzed silk fibroin can be genetically modified to be fused with a protein, e.g., a therapeutic protein. The contents of the aforementioned patent applications are all incorporated herein by reference.

Methods of Producing Silk Fibroin-Based Compositions and/or Emulsion Compositions Described Herein

Methods for producing one or more embodiments of silk fibroin-based compositions and/or emulsion compositions are also provided herein. Accordingly, in one aspect, compositions produced by the methods described herein are provided. In one embodiment, the composition is formulated for use in a personal care product (e.g., a cosmetic, skincare, body care, or hair product). In another embodiment, the composition is formulated for use in a food product.

In general, the silk fibroin-based compositions and/or emulsion compositions in any format described herein (e.g., but not limited to, a gel, a lotion, a serum, a paste) can be produced from a non-hydrolyzed silk fibroin solution and a humectant agent. The

The method of producing a silk fibroin-based composition and/or an emulsion composition described herein comprises mixing a non-hydrolyzed silk fibroin solution and a humectant agent in a volume ratio of about 1:2 to about 1:100. In some embodiments, the volume ratio of the non-hydrolyzed silk fibroin solution to the humectant agent can be about 1:2 to about 1:10. In some embodiments, the volume ratio of the non-hydrolyzed silk fibroin solution to the humectant agent can be about 1:4 to about 1:6. The humectant agent can be powder, a liquid, a solution, or a suspension. Where the humectant agent is powder, the non-hydrolyzed silk fibroin solution and the humectant agent can be mixed in a weight or mass ratio of about 1:1.1 to about 1:1000, about 1:5 to about 1:750, or about 1:10 to about 1:500.

Examples of the humectant agents include, but are not limited to, phospholipid (e.g., lecithin, panthenol (pro-vitamin B5), glycerin (glycerol)), aloe vera, sorbitol, urea, alpha-hydroxy acids, sodium pyroglutamate, N-acetyl-ethanolamine, sodium lactate, isopropanol, polyalkylene glycols (e.g., ethylene glycol, propylene glycol, hexylene glycol, butylene glycol, dipropylene glycol, triethylene glycol), 1,3-propanediol, polyethylene glycols, synthetic alcohols (e.g., glyceryl coconate, hydroxystearate, myristate, oleate), and any combinations thereof. In some embodiments, the humectant agent used in the composition can comprise phospholipid (e.g., but not limited to, lecithin, panthenol (pro-vitamin B5), glycerin), aloe vera, sorbital, alpha-hydroxy acids, or any combinations thereof.

Any desirable concentration of the non-hydrolyzed silk fibroin solution can be used to produce a composition of various aspects described herein. In some embodiments, the non-hydrolyzed silk fibroin solution can have a concentration of about 1% (w/v or w/w) to about 30% (w/v or w/w). In some embodiments, the non-hydrolyzed silk fibroin solution can have a concentration of about 1% (w/v or w/w) to about 10% (w/v or w/w). In some embodiments, the non-hydrolyzed silk fibroin solution can have a concentration of about 5% (w/v or w/w) to about 8% (w/v or w/w).

In some embodiments where the non-hydrolyzed silk fibroin solution has a concentration of about 7% (w/v), it can be desirable to maintain the volume ratio of the non-hydrolyzed silk fibroin solution to glycerin at about 1:4 to about 1:8. The inventors have discovered that silk fibroin maintains a random coil configuration at these ratios. If the amount of glycerin is too high relative to the non-hydrolyzed silk fibroin solution (e.g., a silk: glycerin ratio of about 1:10), the mixture of the non-hydrolyzed silk fibroin solution and glycerin does not appear to form a gel. However, if the amount of glycerin is too low relative to the non-hydrolyzed silk fibroin solution (e.g., a silk: glycerin ratio of about 1:2), the relatively-high concentration of the non-hydrolyzed silk fibroin solution can allow formation of beta sheet structures in small regions within the solution, resulting in precipitation of the protein, especially when exposed to shear forces (such as those produced by rubbing the composition on a skin surface).

In some embodiments, the method can further comprise mixing at least one or more additive described earlier with the humectant agent and the non-hydrolyzed silk fibroin solution. In some embodiments, the aqueous soluble additive can be mixed with the humectant agent prior to mixing with the non-hydrolyzed silk fibroin solution. In some embodiments, the additive mixed with the humectant agent and/or the non-hydrolyzed silk fibroin solution can be aqueous-soluble.

In some embodiments, the method can further comprise mixing an aqueous-based mixture comprising the non-hydrolyzed silk fibroin solution and the humectant agent with an oil-based phase or wax-based phase described herein, for example, to form an emulsion.

In some embodiments, the oil-based phase can further comprise a carrier oil and optionally any art-recognized emulsifier. Any carrier oils known in the art, e.g., for use in formulating personal care products, skin care products, body care products, hair products, and/or aromatherapy products, can be used herein. Examples of carrier oil include, without limitations, a synthetic or natural oil derived from seeds, nuts, fruits, flowers, plant-based materials, or any combinations thereof. Examples of carrier oils include, but are not limited to, sweet almond oil, olive oil, wheat germ oil, castor oil, avocado oil, rosehip seed oil, calendula oil, jojoba oil, grape seed oil, apricot kernel oil, flaxseed oil, hazelnut oil, walnut oil, pecan nut oil, macadamia nut oil, sesame oil, emu oil, coconut oil, and vegetable oils such as for example safflower oil, sunflower oil, canola oil, soybean oil, and peanut oil, and any combinations thereof.

In some embodiments, the wax-based phase can comprise butters, wax, paraffin wax, paraffin oil, petrolatum, or any combinations thereof.

An appropriate ratio of the carrier oil to the emulsifier can be readily determined by a skilled artisan for different emulsion compositions forms. By way of example only, the volume ratio of the carrier oil to the emulsifier can range from about 2:1 to about 100:1.

In some embodiments, the oil-based phase can further comprise an additive described herein. In some embodiments, the additive added into the oil-based phase can be oil- or wax-soluble.

In some embodiments, the silk fibroin based material and/or emulsion compositions can be in the form of a gel or hydrogel. The rate and/or degree of gelation can be controlled by varying the volume or weight/mass ratio of a non-hydrolyzed silk fibroin solution to a humectant agent. By way of example only, when the volume ratio of the non-hydrolyzed silk fibroin solution to glycerin (an example of a humectant agent) is about 1:4, a gel can be formed within seconds, whereas a ratio of 1:8 can form a gel within about 15 minutes. In addition, the 1:4 silk:glycerin formulations can have a more viscous texture, as compared to 1:8 silk:glycerin formulations.

The term “hydrogel” is used herein to mean a silk-based material which exhibits the ability to swell in water and to retain a significant portion of water within its structure without dissolution. Methods for inducing silk fibroin gelation include, but are not limited to, mixing, sonication, vortexing, pH titration, exposure to electric field, solvent immersion, water annealing, water vapor annealing, and the like. Exemplary methods for preparing silk fibroin gels and hydrogels are described in, for example, WO 2005/012606, content of which is incorporated herein by reference in its entirety. By way of example only, a hydrogel can be produced by electrogelation of a non-hydrolyzed silk fibroin solution mixed with a humectant agent, or by altering the pH of the non-hydrolyzed silk fibroin solution via the addition of an acidic or a basic solution to increase or decrease the rate of gelation.

The non-hydrolyzed silk fibroin solution can be prepared by any conventional method known to one skilled in the art. For example, B. mori cocoons are boiled for varying times (e.g., about 10 minutes to about 60 minutes, depending on the form of the silk fibroin-based compositions and/or emulsion compositions to be produced) in an aqueous solution. In one embodiment, the aqueous solution is about 0.02M Na₂CO₃. The cocoons are rinsed, for example, with water to extract the sericin proteins and the extracted non-hydrolyzed silk is dissolved in an aqueous salt solution. Salts useful for this purpose include lithium bromide, lithium thiocyanate, calcium nitrate or other chemicals capable of solubilizing silk. In some embodiments, the extracted non-hydrolyzed silk is dissolved in about 8M-12 M LiBr solution. The salt is consequently removed using, for example, dialysis.

If necessary, the non-hydrolyzed silk fibroin solution can then be concentrated using, for example, dialysis against a hygroscopic polymer solution, for example, PEG, a polyethylene oxide, amylose or sericin. In some embodiments, the PEG is of a molecular weight of 8,000-10,000 g/mol and has a concentration of 5 wt %-50 wt % (e.g., about 15 wt %). A slide-a-lyzer dialysis cassette (Pierce, MW CO 3500) can be used. However, any dialysis system can be used. The dialysis can be performed for a time period sufficient to result in a final concentration of aqueous silk solution between about 10 wt %-about 50 wt %. In some embodiments, the dialysis can be performed for a time period sufficient to result in a final concentration of aqueous silk solution at about 30 wt %. In most cases dialysis for 5-20 hours (e.g., ˜14 hours) is sufficient and longer dialysis is also permitted. See, for example, International Application No. WO 2005/012606, the content of which is incorporated herein by reference.

Alternatively, the non-hydrolyzed silk fibroin solution can be produced using organic solvents. Such methods have been described, for example, in Li, M., et al., J. Appl. Poly Sci. 2001, 79, 2192-2199; Min, S., et al. Sen'I Gakkaishi 1997, 54, 85-92; Nazarov, R. et al., Biomacromolecules 2004 May-June; 5(3):718-26. For example, an exemplary organic solvent that can be used to produce a silk solution includes, but is not limited to, hexafluoroisopropanol.

As silk fibroin can generally stabilize active agents, some embodiments of the silk fibroin-based material can be used to encapsulate and/or deliver an active agent. In these embodiments, at least one active agent can be dispersed into a silk fibroin solution. Non-limiting examples of the active agents can include cells, proteins, peptides, nucleic acids, nucleic acid analogs, nucleotides or oligonucleotides, peptide nucleic acids, aptamers, antibodies or fragments or portions thereof, antigens or epitopes, hormones, hormone antagonists, growth factors or recombinant growth factors and fragments and variants thereof, cell attachment mediators, cytokines, enzymes, antibiotics or antimicrobial compounds, viruses, toxins, therapeutic agents and prodrugs thereof, small molecules, and any combinations thereof.

In some embodiments, at least one additive and/or active agent described herein can be added to the non-hydrolyzed silk fibroin solution before further processing into silk fibroin-based materials or compositions described herein. In some embodiments, the active agent can be dispersed homogeneously or heterogeneously within the silk fibroin, dispersed in a gradient, e.g., using the carbodiimide-mediated modification method described in the U.S. Patent Application No. US 2007/0212730.

In some embodiments, the silk fibroin-based compositions and/or emulsion compositions can be first formed and then contacted with (e.g., dipped into or incubated with) at least one additive and/or active agent. In some embodiments, at least one additive and/or active agent described herein can be coated on an exposed surface of the silk fibroin-based material upon the contacting. In some embodiments, at least one additive and/or active agent described here can diffuse into the silk fibroin-based material upon the contacting.

While it is not always necessary to alter a property of the silk fibroin-based compositions and/or emulsion compositions described herein, post-treatment of the silk fibroin-based composition can be employed, if applicable. For example, to produce a solid composition rather than a flowable gel, post-treatment of the silk fibroin-based compositions can be employed. In some embodiments, beta-sheet formation can also be controlled to produce personal care products and compositions with different aesthetics. For example, post-treatment methods can be applied to the silk fibroin-based material to induce beta-sheet structure formation in silk fibroin and thus modulate physical properties of silk fibroin (e.g., mechanical strength, degradability and/or solubility). Further, such post-treatment to induce formation of beta-sheet conformation structure in silk fibroin can prevent a silk fibroin-based material from contracting into a compact structure and/or forming an entanglement. Examples of various post-treatments can include, without limitations, controlled slow drying (Lu et al., 10 Biomacromolecules 1032 (2009)); water annealing (Jin et al., Water-Stable Silk Films with Reduced Beta-Sheet Content, 15 Adv. Funct. Mats. 1241 (2005); Hu et al. Regulation of Silk Material Structure by Temperature-Controlled Water Vapor Annealing, 12 Biomacromolecules 1686 (2011)); stretching (Demura & Asakura, Immobilization of glucose oxidase with Bombyx mori silk fibroin by only stretching treatment and its application to glucose sensor, 33 Biotech & Bioengin. 598 (1989)); compressing; solvent immersion, including methanol (Hofmann et al., Silk fibroin as an organic polymer for controlled drug delivery, 111 J Control Release. 219 (2006)), ethanol (Miyairi et al., Properties of b-glucosidase immobilized in sericin membrane. 56 J. Fermen. Tech. 303 (1978)), glutaraldehyde (Acharya et al., Performance evaluation of a silk protein-based matrix for the enzymatic conversion of tyrosine to L-DOPA. 3 Biotechnol J. 226 (2008)), and 1-ethyl-3-(3-dimethyl aminopropyl) carbodiimide (EDC) (Bayraktar et al., Silk fibroin as a novel coating material for controlled release of theophylline. 60 Eur J Pharm Biopharm. 373 (2005)); pH adjustment, e.g., pH titration and/or exposing a silk-based material to an electric field (see, e.g., U.S. Patent App. No. US2011/0171239); heat treatment; shear stress (see, e.g., International App. No.: WO 2011/005381), ultrasound, e.g., sonication (see, e.g., U.S. Patent Application Publication No. U.S. 2010/0178304, and International Patent Application No. WO2008/150861); constraint-drying (see, e.g., International Patent Application No. WO 2011/008842); and any combinations thereof. Content of all of the references listed above is incorporated herein by reference in their entirety.

In some embodiments, the silk fibroin-based compositions and/or emulsion compositions described herein can be sterilized. Sterilization methods for biomaterials are well known in the art, including, but not limited to, gamma or ultraviolet radiation, autoclaving (e.g., heat/steam); alcohol sterilization (e.g., ethanol and methanol); and gas sterilization (e.g., ethylene oxide sterilization).

Further, the silk fibroin-based compositions and/or emulsion compositions described herein can take advantage of the many techniques developed to functionalize silk fibroin (e.g., active agents such as dyes and sensors). See, e.g., U.S. Pat. No. 6,287,340, Bioengineered anterior cruciate ligament; WO 2004/000915, Silk Biomaterials & Methods of Use Thereof; WO 2004/001103, Silk Biomaterials & Methods of Use Thereof; WO 2004/062697, Silk Fibroin Materials & Use Thereof; WO 2005/000483, Method for Forming inorganic Coatings; WO 2005/012606, Concentrated Aqueous Silk Fibroin Solution & Use Thereof; WO 2011/005381, Vortex-Induced Silk fibroin Gelation for Encapsulation & Delivery; WO 2005/123114, Silk-Based Drug Delivery System; WO 2006/076711, Fibrous Protein Fusions & Uses Thereof in the Formation of Advanced Organic/Inorganic Composite Materials; U.S. Application Pub. No. 2007/0212730, Covalently immobilized protein gradients in three-dimensional porous scaffolds; WO 2006/042287, Method for Producing Biomaterial Scaffolds; WO 2007/016524, Method for Stepwise Deposition of Silk Fibroin Coatings; WO 2008/085904, Biodegradable Electronic Devices; WO 2008/118133, Silk Microspheres for Encapsulation & Controlled Release; WO 2008/108838, Microfluidic Devices & Methods for Fabricating Same; WO 2008/127404, Nanopatterned Biopolymer Device & Method of Manufacturing Same; WO 2008/118211, Biopolymer Photonic Crystals & Method of Manufacturing Same; WO 2008/127402, Biopolymer Sensor & Method of Manufacturing Same; WO 2008/127403, Biopolymer Optofluidic Device & Method of Manufacturing the Same; WO 2008/127401, Biopolymer Optical Wave Guide & Method of Manufacturing Same; WO 2008/140562, Biopolymer Sensor & Method of Manufacturing Same; WO 2008/127405, Microfluidic Device with Cylindrical Microchannel & Method for Fabricating Same; WO 2008/106485, Tissue-Engineered Silk Organs; WO 2008/140562, Electroactive Biopolymer Optical & Electro-Optical Devices & Method of Manufacturing Same; WO 2008/150861, Method for Silk Fibroin Gelation Using Sonication; WO 2007/103442, Biocompatible Scaffolds & Adipose-Derived Stem Cells; WO 2009/155397, Edible Holographic Silk Products; WO 2009/100280, 3-Dimensional Silk Hydroxyapatite Compositions; WO 2009/061823, Fabrication of Silk Fibroin Photonic Structures by Nanocontact Imprinting; WO 2009/126689, System & Method for Making Biomaterial Structures.

In an alternative embodiment, the silk fibroin-based compositions and/or emulsion compositions described herein can include plasmonic nanoparticles to form photothermal elements, e.g., by adding plasmonic particles into a mixture comprising a non-hydrolyzed silk fibroin solution and a humectant agent and forming the composition therefrom. This approach takes advantage of the superior doping characteristics of silk fibroin. Thermal therapy has been shown to aid in the delivery of various agents, see Park et al., Effect of Heat on Skin Permeability, 359 Intl. J. Pharm. 94 (2008). In one embodiment, short bursts of heat on very limited areas can be used to maximize permeability with minimal harmful effects on surrounding tissues. Thus, plasmonic particle-doped silk fibroin matrices can add specificity to thermal therapy by focusing light to locally generate heat only via the silk fibroin matrices. In some embodiments, the silk fibroin matrices can include photothermal agents such as gold nanoparticles.

### Application of Silk Fibroin-Based Compositions and Emulsion Compositions Described Herein

Yet another aspect provides a method using any compositions of various aspects described herein. The method comprises applying on a target surface one or more embodiments of silk fibroin-based compositions and/or emulsion compositions described herein. The compositions described herein can be applied to a target surface by any means known in the art, e.g., rubbing, spreading, gliding, painting, spraying, extruding, massaging, brushing, smoothing, rolling, or any combinations thereof, depending on the format of the compositions described herein.

By way of example only, when the compositions described herein is flowable or spreadable (e.g., in a form of a serum, a lotion, or a cream), the compositions can be applied on a target surface, e.g., by rubbing, spreading, massaging, smoothing, or any combinations thereof. In other embodiments where the composition and/or emulsion compositions is a solid, e.g., balm, or a stick, the compositions and/or emulsion compositions described herein can be applied by rubbing, rolling, gliding, or any combinations thereof.

Any target surface can be amenable to the application of the compositions described herein. In some embodiments, the target surface can be skin or hair of a subject. In some embodiments, the target surface can be a wound. In some embodiments, the target surface can be a surface of food, for example, where the composition can be used as an edible food-decorating material, e.g., in a form of ink, gel, or paste.

In some embodiments, the method can further comprise maintaining the composition at any temperature above 0° C., e.g., at room temperature or higher, for at least about 1 month or longer, e.g., at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 18 months, at least about 24 months or longer, wherein the composition remains stable afterward (e.g., maintaining at least one property of the composition such as translucency, flowability, silk fibroin protein stability, no brittle gel formation; or no precipitate formation when the composition is exposed to shear).

### Exemplary Personal Care Compositions Comprising Non-Hydrolyzed Silk Fibroin and a Humectant Agent

The silk fibroin-based compositions and/or emulsion compositions described herein can be further formulated accordingly for different types of personal care compositions. In one embodiment, the silk fibroin-based compositions and/or emulsion compositions described herein can be adapted to form a hair care composition in a form selected from the group consisting of shampoo, conditioner, anti-dandruff treatments, styling aids, styling conditioner, hair repair or treatment serum, lotion, cream, pomade, and chemical treatments. In another embodiment, the styling aids are selected from the group consisting of spray, mousse, rinse, gel, foam and a combination thereof. In another embodiment, the chemical treatments are selected from the group consisting of permanent waves, relaxers, and permanent, semi-permanent, and temporary color treatments and combinations thereof.

In another embodiment, the silk fibroin-based compositions and/or emulsion compositions described herein can be adapted to form a skin care composition in a form selected from the group consisting of moisturizing body wash, body wash, antimicrobial cleanser, skin protectant treatment, body lotion, facial cream, moisturizing cream, facial cleansing emulsion, surfactant-based facial cleanser, facial exfoliating gel, facial toner, exfoliating cream, facial mask, after shave balm and sunscreen.

In another embodiment, the silk fibroin-based compositions and/or emulsion compositions described herein can be adapted to form a cosmetic composition in a form selected from the group consisting of eye gel, high-melting point lipstick, lipstick, lip gloss, lip balm, mascara, eyeliner, pressed powder formulation and foundation. In a further embodiment, the cosmetic composition comprises a makeup composition. Makeup compositions include, but are not limited to color cosmetics, such as mascara, lipstick, lip liner, eye shadow, eye liner, rouge, face powder, make up foundation, and nail polish.

In yet another embodiment, the silk fibroin-based compositions and/or emulsion compositions described herein can be adapted to form a nail care composition in a form selected from the group consisting of nail enamel, cuticle treatment, nail polish, nail treatment, and polish remover.

In yet another embodiment, the silk fibroin-based compositions and/or emulsion compositions described herein can be adapted to form an oral care composition in a form selected from the group consisting of toothpaste, mouth rinse, breath freshener, whitening treatment, and inert carrier substrates.

In yet another embodiment, the silk fibroin-based compositions and/or emulsion compositions described herein can be adapted to form an over-the-counter pharmaceutical composition, e.g., sunscreen, anti-acne, antiperspirants, skin protectants, anti-dandruff products, anti-fungal, hemorrhoidal and/or toothpaste.

Moreover, the personal care composition can be in the form of an emulsified vehicle, such as a nutrient cream or lotion, a stabilized gel or dispersioning system, such as skin softener, a nutrient emulsion, a nutrient cream, a massage cream, a treatment serum, a liposomal delivery system, a topical facial pack or mask, a surfactant-based cleansing system such as a shampoo or body wash, an aerosolized or sprayed dispersion or emulsion, a hair or skin conditioner, styling aid, or a pigmented product such as makeup in liquid, cream, solid, anhydrous or pencil form.

In some embodiments of various kinds of the personal care composition described herein, the composition can further comprise an active ingredient or an active agent described herein. One skilled in the art will appreciate the various active ingredients or active agents for use in personal care compositions, any of which may be employed herein, see e.g., McCutcheon's Functional Materials, North American and International Editions, (2003), published by MC Publishing Co. For example, the personal care compositions herein can comprise a skin care active ingredient at a level from about 0.0001% to about 20%, by weight of the composition. In another embodiment, the personal care composition comprises a skin care active ingredient from about 0.001% to about 5%, by weight of the composition. In yet another embodiment, the personal care composition comprises a skin care active ingredient from about 0.01% to about 2%, by weight of the composition.

Skin care active ingredients include, but are not limited to, antioxidants, such as tocopheryl and ascorbyl derivatives; bioflavinoids, terpenoids, synthetics of biolflavinoids and terpenoids and the like; vitamins and vitamin derivatives; hydroxyl- and polyhydroxy acids and their derivatives, such as AHAs and BHAs and their reaction products; peptides and polypeptides and their derivatives, such as glycopeptides and lipophilized peptides, heat shock proteins and cytokines; enzymes and enzymes inhibitors and their derivatives, such as proteases, MMP inhibitors, catalases, CoEnzyme Q10, glucose oxidase and superoxide dismutase (SOD); amino acids and their derivatives; bacterial, fungal and yeast fermentation products and their derivatives, including mushrooms, algae and seaweed and their derivatives; phytosterols and plant and plant part extracts; phospholipids and their derivatives; anti-dandruff agents, such as zinc pyrithione, and sunscreen agents such as ethylhexyl methoxycinnamate, avobenzone, and phenyl benzimidazole sulfonic acid. Delivery systems comprising the active ingredients are also provided herein.

In one embodiment, the skin care active ingredient can comprise a Vitamin B3 component, panthenol, Vitamin E, Vitamin E acetate, retinoid, retinol, retinyl, propionate, retinyl palmitate, retinoic acid, Vitamin C, theobromine, alpha-hydroxyacid, farnesol, phytrantriol, salicylic acid, palmityl peptapeptide-3 and any mixtures thereof. In another embodiment, the Vitamin B3 compound is niacinamide. In yet another embodiment, the vitamin B3 compound is tocopherol nicotinate.

Exemplary derivatives of the foregoing vitamin B3 compounds include nicotinic acid esters, including non-vasodilating esters of nicotinic acid, nicotinyl amino acids, nicotinyl alcohol esters of carboxylic acids, nicotinic acid N-oxide and niacinamide N-oxide. Suitable esters of nicotinic acid include nicotinic acid esters of C1-C22, specifically C1-C16, more specifically C1-C6 alcohols. The alcohols are suitably straight-chain or branched chain, cyclic or acyclic, saturated or unsaturated (including aromatic), and substituted or unsubstituted. The esters are specifically non-vasodilating. Non-vasodilating esters of nicotinic acid include tocopherol nicotinate and inositol hexanicotinate. A more complete description of vitamin B3 compounds is given in WO 98/22085, the content of which is incorporated herein by reference.

The retinoid skin care active ingredient can include, but are not limited to, retinol, retinol esters (e.g., C2-C22 alkyl esters of retinol, including retinyl palmitate, retinyl acetate, retinyl proprionate), retinal, and/or retinoic acid (including all-trans retinoic acid and/or 13-cis-retinoic acid). These compounds are well known in the art and are commercially available from a number of sources, e.g., Sigma Chemical Company, and Boehringer Mannheim. Exemplary retinoids are retinol, retinyl palmitate, retinyl acetate, retinyl proprionate, retinal, retinoic propionate, retinoic acid and combinations thereof. The retinoid can be included as the substantially pure material, or as an extract obtained by suitable physical and/or chemical isolation from natural (e.g., plant) sources. The retinoid may comprise from about 0.005% to about 2% or from about 0.01% to about 12% by weight of the personal care composition. In another embodiment, the personal care composition may comprise retinol. The retinol may comprise from about 0.01% to about 0.15% by weight of the personal care composition. In yet another embodiment, the personal care composition may comprise retinol esters. The retinol esters may comprise from about 0.01% to about 2% by weight of the personal care composition.

In addition to the active ingredients noted above, the personal care composition can further comprise a physiologically acceptable carrier or excipient. Specifically, the personal care compositions herein can comprise a safe and effective amount of a dermatologically acceptable carrier, suitable for topical application to the skin or hair within which the essential materials and optional other materials are incorporated to enable the essential materials and optional components to be delivered to the skin or hair at an appropriate concentration. The carrier can thus act as a diluent, dispersant, solvent or the like for the essential components which ensures that they can be applied to and distributed evenly over the selected target at an appropriate concentration.

An effective amount of one or more compounds described herein can also be included in personal care compositions to be applied to keratinous materials such as nails and hair, including but not limited to those useful as hair spray compositions, hair styling compositions, hair shampooing and/or conditioning compositions, compositions applied for the purpose of hair growth regulation and compositions applied to the hair and scalp for the purpose of treating seborrhea, dermatitis and/or dandruff.

An effective amount of one or more compounds described herein may be included in personal care compositions suitable for topical application to the skin, teeth, nails or hair. These compositions can be in the form of creams, lotions, gels, suspensions dispersions, microemulsions, nanodispersions, microspheres, hydrogels, emulsions (e.g., oil-in-water and water-in-oil, as well as multiple emulsions) and multilaminar gels and the like (see, for example, The Chemistry and Manufacture of Cosmetics, Schlossman et al., 1998), and can be formulated as aqueous or silicone compositions or can be formulated as emulsions of one or more oil phases in an aqueous continuous phase (or an aqueous phase in an oil phase).

While in some embodiments, a humectant agent used in the silk fibroin-based compositions and/or emulsion compositions can also act as a carrier, in some embodiments, the silk fibroin-based compositions and/or emulsion compositions formulated for a personal care composition can further comprise a different carrier. The type of carrier utilized in the personal care compositions can depend on the type of product form desired for the composition. The carrier can be solid, semi-solid or liquid. Suitable carriers are liquid or semi-solid, such as creams, lotions, gels, sticks, wax, ointments, pastes, sprays and mousses. In some embodiments, the carrier is in the form of a lotion, cream or a gel, more specifically one which has a sufficient thickness or yield point to prevent the particles from sedimenting. The carrier can itself be inert or it can possess dermatological benefits of its own. The carrier may be applied directly to the teeth, skin, nails and/or hair or it may be applied via a woven or non-woven wipe or cloth. It may also be in the form of a patch, mask, wrap, or other inert substrate. It may also be aerosolized or otherwise sprayed or pumped onto the skin and/or hair. The carrier should also be physically and chemically compatible with the essential components described herein, and should not unduly impair stability, efficacy or other use benefits associated with the compositions described herein.

In one embodiment, the carrier may be selected from the group consisting of water, propylene glycol, ethanol, propanol, butylene glycol and polyethylene glycol, or a combination thereof. The carriers may also contain a dermatologically acceptable, hydrophilic diluent. Suitable hydrophilic diluents include water, organic hydrophilic diluents such as C2-C10, specifically C2-C6, more specifically, C3-C6 monohydric alcohols and low molecular weight glycols and polyols, including propylene glycol, polyethylene glycol polypropylene glycol, glycerol, butylene glycol, 1,2,4-butanetriol, sorbitol, 1,2,6-hexametriol, pentylene glycol, hexylene glycol, sorbitol esters, ethoxylated ethers, propoxylated ethers and combinations thereof. In one embodiment, the diluent is a liquid. In another embodiment, the diluent is water.

In another embodiment, the personal care composition comprises at least about 20% of the hydrophilic diluent.

The carrier can also include one or more components that facilitate penetration through the upper stratum corneum barrier to the deeper skin layers. Examples of penetration enhancers include, but are not limited to, propylene glycol, azone, ethoxydiglycol, dimethyl isosorbide, urea, ethanol and dimethyl sulfoxide. Other examples include, but are not limited to, micoroemulsions, liposomes and nanoemulsions.

The oil-in-water and oil-in-water-in-oil emulsion compositions described herein can comprise from about 0.05% to about 20%, specifically from about 1% to about 15%, more specifically from about 2% to about 10%, and even more specifically from about 2% to about 5% by weight of a dermatologically acceptable emollient. Emollients tend to lubricate the skin, increase the smoothness and suppleness of the skin, prevent or relieve dryness of the skin and/or protect the skin. Emollients are typically water-immiscible, oily or waxy materials and emollients with high molecular weights can confer aesthetic properties to a topical composition. A wide variety of suitable emollients are known and may be used herein. Sagarin, Cosmetics, Science and Technology, 2nd Edition, Vol. 1, pp. 32-43 (1972), contains numerous examples of materials suitable as an emollient. All emollients discussed in application WO 00/24372 and U.S. Pat. No. 7,297,678, the content of which are incorporated herein by reference, should be considered as suitable for use in the present invention.

Personal care compositions herein can also contain one or more emulsifiers and/or surfactants, generally to help disperse and suspend the disperse phase within the continuous phase. A surfactant may be useful if the product is intended for skin or hair cleansing. For convenience hereinafter emulsifiers will be referred to under the term “surfactants”. Thus, surfactant(s) will be used to refer to surface active agents whether used as emulsifiers or for other surfactant purposes such as skin cleansing. Known or conventional surfactants can be used in the composition, provided that the selected agent is chemically and physically compatible with essential components of the composition and provides the desired characteristics. Suitable surfactants include non-silicone derived materials, silicone-derived materials, and mixtures thereof. All surfactants discussed in application WO 00/24372 should be considered as suitable for use in the present invention. The personal care compositions of the present invention may comprise from about 0.05% to about 30%, specifically from about 0.5% to 15%, and more specifically from about 1% to 10% by weight of a surfactant or mixture of surfactants. The exact surfactant or surfactant mixture chosen will depend upon the pH of the composition, the other components present and the desired final product aesthetics.

Among the nonionic surfactants that are useful herein are those that can be broadly defined as condensation products of long chain alcohols, e.g. C8-30 alcohols, with sugar or starch polymers, i.e., glycosides. Other useful nonionic surfactants include the condensation products of alkylene oxides with fatty acids (i.e., alkylene oxide esters of fatty acids). These materials have the general formula RCO(X)nOH wherein R is a C10-30 alkyl group, X is —OCH2CH2- (i.e., derived from ethylene glycol or oxide) or —OCH2CHCH3- (i.e., derived from propylene glycol or oxide) and n is an integer from 6 to 200. Other nonionic surfactants are the condensation products of alkylene oxides with 2 moles of fatty acids (i.e., alkylene oxide diesters of fatty acids). These materials have the general formula RCO(X)nOOCR wherein R is a C10-30 alkyl group, X is —OCH2CH2- (i.e., derived from ethylene glycol or oxide) or —OCH2CHCH3- (i.e., derived from propylene glycol or oxide) and n is an integer from 6 to 100. For example, an emulsifier for use herein is most specifically a fatty acid ester blend based on a mixture of sorbitan fatty acid ester and sucrose fatty acid ester, more specifically a blend of sorbitan stearate and sucrose cocoate. Even further suitable examples include a mixture of cetearyl alcohols and cetearyl glucosides.

The hydrophilic surfactants useful herein can alternatively or additionally include any of a wide variety of cationic, anionic, zwitterionic, and amphoteric surfactants such as are known in the art. (See, e.g., McCutcheon's, Detergents and Emulsifiers and Detergents, North American (2003) and International Editions (1986), published by MC Publishing Co. and Allured Publishing Corporation; U.S. Pat. No. 5,011,681 to Ciotti et al., U.S. Pat. No. 4,421,769 to Dixon et al.; and U.S. Pat. No. 3,755,560 to Dickert et al.).

A variety of anionic surfactants are also useful herein. (See, e.g., U.S. Pat. No. 3,929,678, to Laughlin et al.). Examples of anionic surfactants include the alkoyl isethionates (e.g., C12-C30), alkyl and alkyl ether sulfates and salts thereof, alkyl and alkyl ether phosphates and salts thereof, alkyl methyl taurates (e.g., C12-C30), and soaps (e.g., substituted alkylamine and alkali metal salts, e.g., sodium or potassium salts) of fatty acids.

Amphoteric and zwitterionic surfactants are also useful herein. Examples of amphoteric and zwitterionic surfactants which can be used in the compositions of the present invention are those which are broadly described as derivatives of aliphatic secondary and tertiary amines in which the aliphatic radical can be straight or branched chain and wherein one of the aliphatic substituents contains from about 8 to about 22 carbon atoms (specifically C8-C18) and one contains an anionic water solubilizing group, e.g., carboxy, sulfonate, sulfate, phosphate, or phosphonate. Examples are alkyl imino acetates and iminodialkanoates and aminoalkanoates, imidazolinium and ammonium derivatives. Other suitable amphoteric and zwitterionic surfactants are those selected from the group consisting of betaines, sultaines, hydroxysultaines, and branched and unbranched alkanoyl sarcosinates, and mixtures thereof.

Some emulsion compositions described herein can include a silicone containing emulsifier or surfactant. A wide variety of silicone emulsifiers are useful herein. These silicone emulsifiers are typically organically modified organopolysiloxanes, also known to those skilled in the art as silicone surfactants. Useful silicone emulsifiers include dimethicone copolyols. These materials are polydimethyl siloxanes which have been modified to include polyether side chains such as polyethylene oxide chains, polypropylene oxide chains, mixtures of these chains and polyether chains containing moieties derived from both ethylene oxide and propylene oxide. Other examples include alkyl-modified dimethicone copolyols, i.e., compounds which contain C2-C30 pendant side chains. Still other useful dimethicone copolyols include materials having various cationic, anionic, amphoteric, and zwitterionic pendant moieties.

The personal care compositions described herein may further comprise at least one polymeric thickening agent. The polymeric thickening agents useful herein may have a number average molecular weight of greater than about 20,000, specifically greater than about 50,000, and more specifically greater than about 100,000. The personal care compositions of the present invention may comprise from about 0.01% to about 10%, specifically from about 0.1% to about 8%, and more specifically from about 0.25% to about 5% by weight of a polymeric thickening agent or mixtures thereof.

Examples of polymer thickening agents for use herein include non-ionic thickening agents and anionic thickening agents or mixtures thereof. Suitable non-ionic thickening agents include polyacrylamide polymers, crosslinked poly(N-vinylpyrrolidones), polysaccharides, natural or synthetic gums, polyvinylpyrrolidone and polyvinylalcohol. Suitable anionic thickening agents include acrylic acid/ethyl acrylate copolymers, carboxyvinyl polymers and crosslinked copolymers of alkyl vinyl ethers and maleic anhydride. As an example, Noveon sells a thickener under the trademark of CARBOPOL™ resins or mixtures thereof. Suitable Carbopol resins may be hydrophobically modified, and other suitable resins are described in WO98/22085, or mixtures thereof.

In some embodiments, the personal care compositions can comprise at least one silicone oil phase. Silicone oil phase(s) generally comprises from about 0.1% to about 20%, specifically from about 0.5% to about 10%, and more specifically from about 0.5% to about 5% by weight of the composition. The silicone oil phase may comprise one or more silicone components.

Silicone components can be fluids, including straight chain, branched and cyclic silicones. Suitable silicone fluids useful herein include silicones inclusive of polyalkyl siloxane fluids, polyaryl siloxane fluids, cyclic and linear polyalkylsiloxanes, polyalkoxylated silicones, amino and quaternary ammonium modified silicones, polyalkylaryl siloxanes or a polyether siloxane copolymer and mixtures thereof. The silicone fluids can be volatile or non-volatile. Silicone fluids generally have an average molecular weight of less than about 200,000. Suitable silicone fluids have a molecular weight of about 100,000 or less, specifically about 50,000 or less, and more specifically about 10,000 or less. Particularly, the silicone fluid is selected from silicone fluids having a weight average molecular weight in the range from about 100 to about 50,000 and specifically from about 200 to about 40,000.

Typically, silicone fluids have a viscosity ranging from about 0.65 to about 600,000 mm² s⁻¹, specifically from about 0.65 to about 10,000 mm² s⁻¹ at 25° C. The viscosity can be measured by means of a glass capillary viscometer. Suitable polydimethyl siloxanes that can be used herein include those available, for example, from the General Electric Company and from Dow Corning. Also useful are essentially non-volatile polyalkylarylsiloxanes, for example, polymethylphenylsiloxanes, having viscosities of about 0.65 to 30,000 mm² s⁻¹ at 25° C. These siloxanes are available, for example, from the General Electric Company or from Dow Corning. Cyclic polydimethylsiloxanes suitable for use herein are those having a ring structure incorporating from about 3 to about 7 (CH₃)₂SiO moieties, specifically about 5 or more.

Silicone gums may also be used herein. In specific embodiments, a silicone oil phase comprises a silicone gum or a mixture of silicones including the silicone gum. Typically, silicone gums have a viscosity at 25° C. in excess of about 1,000,000 mm² s⁻¹. The silicone gums include dimethicones as described by Petrarch and others including U.S. Pat. No. 4,152,416, May 1, 1979 to Spitzer, et al, and Noll, Walter, Chemistry and Technology of Silicones, New York: Academic Press 1968. Also describing silicone gums are General Electric Silicone Rubber Product Data Sheets SE 30, SE 33, SE 54 and SE 76. Specific examples of silicone gums include polydimethylsiloxane, (polydimethylsiloxane)(methylvinylsiloxane) copolymer, poly(dimethylsiloxane)(diphenyl)(methylvinylsiloxane) copolymer and mixtures thereof. Preferred silicone gums for use herein are silicone gums having a molecular weight of from about 200,000 to about 4,000,000 selected from dimethiconol, dimethicone copolyol, dimethicone and mixtures thereof.

A silicone phase herein may comprise a silicone gum incorporated into the composition as part of a silicone gum-fluid blend. When the silicone gum is incorporated as part of a silicone gum-fluid blend, the silicone gum may constitute from about 5% to about 40% and specifically from about 10% to 20% by weight of the silicone gum-fluid blend.

Further silicone components suitable for use in a silicone oil phase herein are crosslinked polyorganosiloxane polymers, optionally dispersed in a fluid carrier. In general, when present the crosslinked polyorganosiloxane polymers, together with their carrier (if present) comprise from about 0.1% to about 20%, specifically from about 0.5% to about 10%, and more specifically from about 0.5% to about 5% by weight of the personal care composition. Such polymers comprise polyorganosiloxane polymers crosslinked by a crosslinking agent. Suitable crosslinking agents are disclosed in WO98/22085. Examples of suitable polyorganosiloxane polymers for use herein include methyl vinyl dimethicone, methyl vinyl diphenyl dimethicone and methyl vinyl phenyl methyl diphenyl dimethicone.

Another class of silicone components suitable for use in a silicone oil phase herein includes polydiorganosiloxane-polyoxyalkylene copolymers containing at least one polydiorganosiloxane segment and at least one polyoxyalkylene segment. Suitable polydiorganosiloxane segments and copolymers thereof are disclosed in WO98/22085. Suitable polydiorganosiloxane-polyalkylene copolymers are available commercially under the tradename BELSIL™ from Wacker-Chemie GmbH. An example of a copolymer fluid blend for use herein includes Dow Corning DC3225C which has the CTFA designation Dimethicone/Dimethicone copolyol.

Personal care compositions described herein can also comprise an organic sunscreen. Suitable sunscreens can have UVA absorbing properties, UVB absorbing properties or a mixture thereof. The exact amount of the sunscreen active will vary depending upon the desired Sun Protection Factor, i.e., the “SPF” of the composition as well as the desired level of UV protection. SPF is a commonly used measure of photoprotection of a sunscreen against erythema. The SPF is defined as a ratio of the ultraviolet energy required to produce minimal erythema on protected skin to that required to produce the same minimal erythema on unprotected skin in the same individual. Amounts of the sunscreen may comprise from about 2% to about 20%, and specifically from about 4% to about 14% by weight of the personal care composition. Suitable sunscreens include, but are not limited to, those approved for use in the United States, Japan, Europe and Australia. The compositions described herein can comprise an SPF of about 2 to about 100, or about 4 about 70, or about 4 to about 30.

The personal care compositions described herein can include one or more UVA absorbing sunscreen actives that absorb UV radiation having a wavelength of from about 320 nm to about 400 nm. Suitable UVA absorbing sunscreen actives are selected from dibenzoylmethane derivatives, anthranilate derivatives such as methylanthranilate and homomethyl, 1-N-acetylanthranilate, and mixtures thereof. Examples of dibenzoylmethane sunscreen actives are described in Sunscreens: Development, Evaluation, and Regulatory Aspects edited by N. J. Lowe and N. A. Shaath, Marcel Dekker, Inc (1990). The UVA absorbing sunscreen active may be present in an amount to provide broad spectrum UVA protection either independently, or in combination with, other UV protective actives which may be present in the composition.

Suitable UVA sunscreen actives are dibenzoylmethane sunscreen actives and their derivatives. They include, but are not limited to, those selected from 2-methyldibenzoylmethane, 4-methyldibenzoylmethane, 4-isopropyldibenzoylmethane, 4-tert-butyldibenzoylmethane, 2,4-dimethyldibenzoylmethane, 2,5-dimethyldibenzoylmethane, 4,4′-diisopropylbenzoylmethane, 4-(1,1-dimethylethyl)-4′-methoxydibenzoylmethane, 2-methyl-5-isopropyl-4′-methoxydibenzoylmethane, 2-methyl-5-tert-butyl-4′-methoxydibenzoylmethane, 2,4-dimethyl-4′-methoxydibenzoylmethane, 2,6-dimethyl-4′-tert-butyl-4′-methoxydibenzoylmethane, and mixtures thereof. In one embodiment, the dibenzoyl sunscreen actives include those selected from 4-(1,1-dimethylethyl)-4′-methoxydibenzoylmethane, 4-isopropyldibenzoylmethane, and mixtures thereof. In another embodiment, the sunscreen active is 4-(1,1-dimethylethyl)-4′-methoxydibenzoylmethane.

The sunscreen active 4-(1,1-dimethylethyl)-4′-methoxydibenzoylmethane, which is also known as butyl methoxydibenzoylmethane or Avobenzone, is commercially available under the names of PARSOL® 1789 from Givaudan Roure (International) S. A. and EUSOLEX® 9020 from Merck & Co., Inc. The sunscreen 4-isoproplydibenzoylmethane, which is also known as isopropyldibenzoylmethane, is commercially available from Merck under the name of EUSOLEX® 8020.

The personal care compositions described herein can further include one or more UVB sunscreen actives that absorb UV radiation having a wavelength of from about 290 nm to about 320 nm. The compositions comprise an amount of the UVB sunscreen active that which is safe and effective to provide UVB protection either independently, or in combination with, other UV protective actives which may be present in the compositions. The compositions may comprise from about 0.1% to about 20%, specifically from about 0.1% to about 12%, and more specifically from about 0.5% to about 8% by weight of each UVB absorbing organic sunscreen, or as mandated by the relevant regulatory authority(s).

A variety of UVB sunscreen actives are suitable for use herein. Non-limiting examples of such organic sunscreen actives are described in U.S. Pat. No. 5,087,372 to Haffey et al; and U.S. Pat. Nos. 5,073,371 and 5,073,372 to Turner et al. Still other useful sunscreens are those disclosed in U.S. Pat. No. 4,937,370, to Sabatelli; and U.S. Pat. No. 4,999,186, to Sabatelli et al. Preferred UVB sunscreen actives are selected from 2-ethylhexyl-2-cyano-3,2-ethylhexyl N,N-dimethyl-p-aminobenzoate, p-aminobenzoic acid, oxybenzone, homomethyl salicylate, octyl salicylate, 4,4′-methoxy-t-butyldibenzoylmethane, 4-isopropyl dibenzoylmethane, 3-benzylidene camphor, 3-(4-methylbenzylidene) camphor, 3-diphenylacrylate, 2-phenyl-benzimidazole-5-sulphonic acid (PBSA), cinnamate esters and their derivatives such as 2-ethylhexyl-p-methoxycinnamate and octyl-p-methoxycinnamate, salicylate esters and their derivatives such as TEA triethanolamine salicylate, ethylhexyl saliycyilate, octyldimethyl para-aminobenzoic acidPABA, camphor derivatives and their derivatives, and mixtures thereof. Examples of organic sunscreen actives are 2-ethylhexyl-2-cyano-3,3-diphenylacrylate, 2-phenyl-benzimidazole-5-sulphonic acid (PBSA), octyl-p-methoxycinnamate, and mixtures thereof. Salt and acid neutralized forms of the acidic sunscreens are also useful herein.

An agent may also be added to any of the compositions described herein to stabilize the UVA sunscreen to prevent it from photo-degrading on exposure to UV radiation and thereby maintaining its UVA protection efficacy. A wide range of compounds have been cited as providing these stabilizing properties and should be chosen to complement both the UVA sunscreen and the composition as a whole. Suitable stabilizing agents include, but are not limited to, those described in U.S. Pat. Nos. 5,972,316; 5,968,485; 5,935,556; 5,827,508 and Published International Application WO 00/06110. Examples of stabilizing agents for use in the present invention include 2-ethylhexyl-2-cyano-3,3-diphenylacrylate, ethyl-2-cyano-3,3-diphenylacrylate, 2-ethylhexyl-3,3-diphenylacrylate, ethyl-3,3-bis(4-methoxyphenyl)acrylate, diethylhexyl 2,6 napthalate and mixtures thereof (Symrise Chemical Company).

An agent may also be added to any of the personal care compositions described herein to improve the skin substantivity of those compositions, particularly to enhance their resistance to being washed off by water or rubbed off. Examples include, but are not limited to, acrylates/C12-22 alkylmethacrylate copolymer, acrylate/acrylate copolymer, dimethicone, dimethiconol, graft-copoly (dimethylsiloxane/iI-butyl methacrylate), lauryl dimethicone, PVP/Hexadecane copolymer, PVP/Eicosene copolymer, tricontanyl PVP and trimethoxysiloxysiliacate.

In addition to the organic sunscreens, personal care compositions described herein can additionally comprise inorganic physical sunblocks. Non-limiting examples of suitable physical sunblocks are described in CTFA International Cosmetic Ingredient Dictionary, 6th Edition, 1995, pp. 1026-28 and 1103, Sayre, R. M. et al, “Physical Sunscreens”, J. Soc. Cosmet. Chem., vol 41, no 2, pp. 103-109 (1990) and Lowe et al., as per above. Specific examples of inorganic physical sunblocks are zinc oxide and titanium dioxide and mixtures thereof.

When used, the physical sunblocks are present in an amount such that the present compositions are transparent on the skin (i.e., non-whitening), from about 0.5% to about 20%, or from about 0.51% to about 10%, or from about 0.5% to 5% by weight of the composition. When titanium dioxide is used, it can have an anatase, rutile or amorphous structure. Manufacturers of micronized grade titanium dioxide and zinc oxide for sunscreen use include, but are not limited to Tayca Corporation, Uniqema, Shinetsu Chemical Corporation, Ken-McGee, Nanophase, Nanosource, Sachtleben, Elementis, and BASF Corporation, as well as their distribution agents and those companies that further process the material for sunscreen use. Physical sunblock particles, e.g., titanium dioxide and zinc oxide, can be uncoated or coated with a variety of materials including but not limited to amino acids, aluminium compounds such as alumina, aluminium stearate, aluminium laurate, and the like; carboxylic acids and their salts e.g., stearic acid and its salts; phospholipids, such as lecithin; organic silicon compounds; inorganic silicon compounds such as silica and silicates and mixtures thereof. The personal care compositions described herein can comprise from about 0.1% to about 15%, or from about 0.1% to about 7% or from about 0.5% to about 5% by weight of an inorganic sunscreen.

The personal care compositions described herein can also include preservatives. Such preservatives include, but are not limited to pentylene glycol, ethylene diamine tetra acetate (also known as EDTA) and its salts, chlorhexidine (and its diacetate, dihydrochloride, digluconate derivatives), 1,1,1-trichloro-2-methyl-2-propanol, parachloro metaxylenol, polyhexamethylenebiguanide hydrochloride, dehydroacetic acid, diazolidinyl urea, 2,4-dichlorobenzyl alcohol, 4,4-dimethyl-1,3-oxazolidine, formaldehyde, glutaraldehyde, dimethylidantoin, imidazolidinyl urea, 5-chloro-2-methyl-4-isothiazolin-3-one, ortho-phenylphenol, 4-hydroxybenzoic acid and its (methyl-, ethyl-, propyl-, isopropyl-, butyl-, isobutyl-) esters (also known as parabens), salts, trichlosan, 2-phenoxyethanol, phenyl mercuric acetate, borate, nitrate, quaternium-15, salicilate, salicylic acid and its salts, calcium, sorbic acid and its salts, iodopropanyl butylcarbamate, calcium sorbate, zinc pyrithione, benzyl alcohol, 5-bromo-Snitro-1,3-dioxane, 2-bromo-2-nitropropane-1,3-diol, benzoic acid and its salts, sulfites, bisulfites, and benzalkonium chloride, phenoxyethanol and chloroxylenol, diazolidinyl urea, methylparaben, propylparaben, PG, isopropylparabens, isobutylparabens, butylparabens, ethylparaben, phenoxyethanol.

A variety of optional ingredients such as neutralizing agents, fragrance, perfumes and perfume solubilizing agents, and coloring agents, can also be added to the personal care compositions herein. Any additional ingredients should enhance the product, for example, the skin softness/smoothness benefits of the product. In addition, any such ingredients should not negatively impact the aesthetic properties of the product. Neutralizing agents suitable for use in neutralizing acidic group containing hydrophilic gelling agents herein include sodium hydroxide, potassium hydroxide, ammonium hydroxide, monoethanolamine, diethanolamine, amino methyl propanol, tris-buffer and triethanolamine.

Other optional materials include any of the various functional and/or active ingredients known to those skilled in the art. (See e.g., McCutcheon's Functional Materials, North American and International Editions, (2003), published by MC Publishing Co.) Non-limiting examples include: keratolytic agents; water-soluble or solubilizable preservatives specifically at a level of from about 0.1% to about 5%, such as Germall 115, methyl, ethyl, propyl and butyl esters of hydroxybenzoic acid, benzyl alcohol, DMDM hydantoin iodopropanyl butylcarbanate available under the trade name Glydant Plus from Lonza, EDTA, Euxyl (RTM) K400, Bromopol (2-bromo-2-nitropropane-1,3-diol) and phenoxypropanol; anti-bacterials such as Irgasan (RTM) and phenoxyethanol (specifically at levels of from about 0.1% to about 5%); soluble or colloidally-soluble moisturizing agents such as hyaluaronic acid and chondroitin sulfatestarch-grafted sodium polyacrylates such as Sanwet (RTM) 1M-1000, IM-1500 and 1M-2500 available from Celanese Superabsorbent Materials, Portsmith, Va., USA and described in U.S. Pat. No. 4,076,663; vitamins such as vitamin A, vitamin C, vitamin E, vitamin K and derivatives thereof and building blocks thereof; such as phytantriol; and vitamin K and components thereof such as the fatty alcohols such as dodecatrienol; alpha and beta hydroxyacids; aloe vera; sphingosines and phytosphingosines, cholesterol; skin whitening agents; N-acetyl cysteine; colouring agents; antibacterial agents such as TCC/TCS, also known as triclosan and trichlorocarbon; perfumes and perfume solubilizers. Examples of alpha hydroxy acids include glycolic acid, lactic acid, malic acid, and citric acid (whether derived synthetically or from natural sources and whether used alone or in combination), and their esters or relevant buffered combinations such as glycolic acid in conjunction with ammonium glycolate. Other examples of alpha-hydroxy acids include: alpha-hydroxy ethanoic acid, alpha-hydroxyoctanoic acid, alpha-hydroxycaprylic acid, and hydroxycaprylic acid, mixed fruit acid, tri-alpha hydroxy fruit acids, triple fruit acid, sugar cane extract, alpha hydroxy and botanical comprise, 1-alpha hydroxy acid and glycomer in crosslinked fatty acids alpha nutrium. Specific examples of alpha hydroxy acids are glycolic acid and lactic acid. In a particular embodiment, alpha hydroxy acids are used in levels of up to about 10%.

Optional materials include pigments that, where water-insoluble, contribute to and are included in the total level of oil phase ingredients. Pigments suitable for use in the compositions described herein can be organic and/or inorganic. Also included within the term pigment are materials having a low color or luster, such as matte finishing agents, light scattering agents, and formulation aids such as micas, seracites, and carbonate salts. Further examples of suitable pigments are titanium dioxide, predispersed titanium dioxide, iron oxides, zinc oxide, bismuth oxychloride (whether pre-dispersed and/or pre-coated or not) coated iron oxides, ultramarine blue, D&C dyes and lakes, FD&C colors, natural color additives such as carmine, and mixtures thereof. Depending upon the type of composition, a mixture of pigments will normally be used. Exemplary pigments for use herein from the viewpoint of moisturization, skin feel, skin appearance and emulsion compatibility are treated pigments. The pigments may be treated with compounds, including but not limited to amino acids, silicones, lecithin and ester oils.

Suitably, the pH of the personal care compositions herein is in the range from about 3.5 to about 10, specifically from about 4 to about 8, and more specifically from about 5 to about 7, wherein the pH of the final composition is adjusted by addition of acidic, basic or buffer salts as necessary, depending upon the composition of the forms and the pH-requirements of the compounds.

One skilled in the art will appreciate the various techniques for preparing the personal care compositions of the present invention, any of which may be employed herein. In general the aqueous phase and/or the oil phase can be prepared separately, with materials of similar phase partitioning being added in any order. If the final product is an emulsion, the two phases can then be combined with vigorous stirring and/or homogenization as necessary to reduce the size of the internal phase droplets. Any ingredients in the formulation with high volatility, or which are susceptible to hydrolysis or decomposition at high temperatures, can be added with gentle stirring towards the end of the process, post emulsification if applicable. In some embodiments, the personal care compositions described herein can be produced by the methods of producing silk fibroin-based compositions and/or emulsion compositions described herein. Dosage frequency and amount generally depend upon the desired performance criteria.

### Additional Exemplary Active Agents

An active agent that can be included in a silk fibroin-based composition and/or emulsion compositions can represent any material capable of being incorporated in a silk fibroin-based material. For example, the active agent can be a therapeutic agent, or a biological material, such as cells (including stem cells such as induced pluripotent stem cells), proteins, peptides, nucleic acids (e.g., DNA, RNA, siRNA), nucleic acid analogs, nucleotides, oligonucleotides, peptide nucleic acids (PNA), aptamers, antibodies or fragments or portions thereof (e.g., paratopes or complementarity-determining regions), antigens or epitopes, hormones, hormone antagonists, growth factors or recombinant growth factors and fragments and variants thereof, cell attachment mediators (such as RGD), cytokines, enzymes, small molecules, antibiotics or antimicrobial compounds, viruses, antivirals, toxins, therapeutic agents and prodrugs, small molecules and any combinations thereof. See, e.g., WO 2009/140588; U.S. Patent Application Ser. No. 61/224,618). The active agent can also be a combination of any of the above-mentioned agents. Encapsulating either a therapeutic agent or biological material, or the combination of them, is desirous because the encapsulated composition can be used for numerous biomedical purposes.

In some embodiments, the active agent can also be an organism such as a fungus, plant, animal, bacterium, or a virus (including bacteriophage). Moreover, the active agent may include neurotransmitters, hormones, intracellular signal transduction agents, pharmaceutically active agents, toxic agents, agricultural chemicals, chemical toxins, biological toxins, microbes, and animal cells such as neurons, liver cells, and immune system cells. The active agents may also include therapeutic compounds, such as pharmacological materials, vitamins, sedatives, hypnotics, prostaglandins and radiopharmaceuticals.

Exemplary cells suitable for use herein may include, but are not limited to, nucleated cells (e.g., single-nucleated cells or multi-nucleated cells), non-nucleated cells such as blood cells (e.g., red blood cells, and platelets), progenitor cells or stem cells, smooth muscle cells, skeletal muscle cells, cardiac muscle cells, epithelial cells, endothelial cells, urothelial cells, fibroblasts, myoblasts, ocular cells, chondrocytes, chondroblasts, osteoblasts, osteoclasts, keratinocytes, kidney tubular cells, kidney basement membrane cells, integumentary cells, bone marrow cells, hepatocytes, bile duct cells, pancreatic islet cells, thyroid, parathyroid, adrenal, hypothalamic, pituitary, ovarian, testicular, salivary gland cells, adipocytes, and precursor cells. The active agents can also be the combinations of any of the cells listed above. See also WO 2008/106485; WO 2010/040129; WO 2007/103442.

As used herein, the terms “proteins” and “peptides” are used interchangeably herein to designate a series of amino acid residues connected to the other by peptide bonds between the alpha-amino and carboxy groups of adjacent residues. The terms “protein”, and “peptide”, which are used interchangeably herein, refer to a polymer of protein amino acids, including modified amino acids (e.g., phosphorylated, glycated, etc.) and amino acid analogs, regardless of its size or function. Although “protein” is often used in reference to relatively large polypeptides, and “peptide” is often used in reference to small polypeptides, usage of these terms in the art overlaps and varies. The term “peptide” as used herein refers to peptides, polypeptides, proteins and fragments of proteins, unless otherwise noted. The terms “protein” and “peptide” are used interchangeably herein when referring to a gene product and fragments thereof. Thus, exemplary peptides or proteins include gene products, naturally occurring proteins, homologs, orthologs, paralogs, fragments and other equivalents, variants, fragments, and analogs of the foregoing.

The term “nucleic acids” used herein refers to polynucleotides such as deoxyribonucleic acid (DNA), and, where appropriate, ribonucleic acid (RNA), polymers thereof in either single- or double-stranded form. Unless specifically limited, the term encompasses nucleic acids containing known analogs of natural nucleotides, which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer, et al., Nucleic Acid Res. 19:5081 (1991); Ohtsuka, et al., J. Biol. Chem. 260:2605-2608 (1985), and Rossolini, et al., Mol. Cell. Probes 8:91-98 (1994)). The term “nucleic acid” should also be understood to include, as equivalents, derivatives, variants and analogs of either RNA or DNA made from nucleotide analogs, and, single (sense or antisense) and double-stranded polynucleotides. The term “nucleic acid” also encompasses modified RNA (modRNA). The term “nucleic acid” also encompasses siRNA, shRNA or any combinations thereof.

The term “modified RNA” means that at least a portion of the RNA has been modified, e.g., in its ribose unit, in its nitrogenous base, in its internucleoside linkage group, or any combinations thereof. Accordingly, in some embodiments, a “modified RNA” may contain a sugar moiety which differs from ribose, such as a ribose monomer where the 2′-OH group has been modified. Alternatively, or in addition to being modified at its ribose unit, a “modified RNA” may contain a nitrogenous base which differs from A, C, G and U (a “non-RNA nucleobase”), such as T or MeC. In some embodiments, a “modified RNA” may contain an internucleoside linkage group which is different from phosphate (—O—P(O)₂—O—), such as —O—P(O,S)—O—.

The term “short interfering RNA” (siRNA), also referred to herein as “small interfering RNA” is defined as an agent which functions to inhibit expression of a target gene, e.g., by RNAi. An siRNA can be chemically synthesized, it can be produced by in vitro transcription, or it can be produced within a host cell. siRNA molecules can also be generated by cleavage of double stranded RNA, where one strand is identical to the message to be inactivated. The term “siRNA” refers to small inhibitory RNA duplexes that induce the RNA interference (RNAi) pathway. These molecules can vary in length (generally 18-30 base pairs) and contain varying degrees of complementarity to their target mRNA in the antisense strand. Some, but not all, siRNA have unpaired overhanging bases on the 5′ or 3′ end of the sense 60 strand and/or the antisense strand. The term “siRNA” includes duplexes of two separate strands, as well as single strands that can form hairpin structures comprising a duplex region.

The term “shRNA” as used herein refers to short hairpin RNA which functions as RNAi and/or siRNA species but differs in that shRNA species are double stranded hairpin-like structure for increased stability. The term “RNAi” as used herein refers to interfering RNA, or RNA interference molecules are nucleic acid molecules or analogues thereof for example RNA-based molecules that inhibit gene expression. RNAi refers to a means of selective post-transcriptional gene silencing. RNAi can result in the destruction of specific mRNA, or prevents the processing or translation of RNA, such as mRNA.

The term “enzymes” as used here refers to a protein molecule that catalyzes chemical reactions of other substances without it being destroyed or substantially altered upon completion of the reactions. The term can include naturally occurring enzymes and bioengineered enzymes or mixtures thereof. Examples of enzyme families include, but are not limited to, peroxidase, lipase, amylose, organophosphate dehydrogenase, ligases, restriction endonucleases, ribonucleases, DNA polymerases, glucose oxidase, laccase, kinases, dehydrogenases, oxidoreductases, GTPases, carboxyl transferases, acyl transferases, decarboxylases, transaminases, racemases, methyl transferases, formyl transferases, and α-ketodecarboxylases.

As used herein, the term “aptamers” means a single-stranded, partially single-stranded, partially double-stranded or double-stranded nucleotide sequence capable of specifically recognizing a selected non-oligonucleotide molecule or group of molecules. In some embodiments, the aptamer recognizes the non-oligonucleotide molecule or group of molecules by a mechanism other than Watson-Crick base pairing or triplex formation. Aptamers can include, without limitation, defined sequence segments and sequences comprising nucleotides, ribonucleotides, deoxyribonucleotides, nucleotide analogs, modified nucleotides and nucleotides comprising backbone modifications, branchpoints and normucleotide residues, groups or bridges. Methods for selecting aptamers for binding to a molecule are widely known in the art and easily accessible to one of ordinary skill in the art.

As used herein, the term “antibody” or “antibodies” refers to an intact immunoglobulin or to a monoclonal or polyclonal antigen-binding fragment with the Fc (crystallizable fragment) region or FcRn binding fragment of the Fc region. The term “antibodies” also includes “antibody-like molecules”, such as fragments of the antibodies, e.g., antigen-binding fragments. Antigen-binding fragments can be produced by recombinant DNA techniques or by enzymatic or chemical cleavage of intact antibodies. “Antigen-binding fragments” include, inter alia, Fab, Fab′, F(ab′)2, Fv, dAb, and complementarity determining region (CDR) fragments, single-chain antibodies (scFv), single domain antibodies, chimeric antibodies, diabodies, and polypeptides that contain at least a portion of an immunoglobulin that is sufficient to confer specific antigen binding to the polypeptide. Linear antibodies are also included for the purposes described herein. The terms Fab, Fc, pFc′, F(ab′) 2 and Fv are employed with standard immunological meanings (Klein, Immunology (John Wiley, New York, N.Y., 1982); Clark, W. R. (1986) The Experimental Foundations of Modern Immunology (Wiley & Sons, Inc., New York); and Roitt, I. (1991) Essential Immunology, 7th Ed., (Blackwell Scientific Publications, Oxford)). Antibodies or antigen-binding fragments specific for various antigens are available commercially from vendors such as R&D Systems, BD Biosciences, e-Biosciences and Miltenyi, or can be raised against these cell-surface markers by methods known to those skilled in the art.

Exemplary antibodies that may be incorporated in silk fibroin include, but are not limited to, abciximab, adalimumab, alemtuzumab, basiliximab, bevacizumab, cetuximab, certolizumab pegol, daclizumab, eculizumab, efalizumab, gemtuzumab, ibritumomab tiuxetan, infliximab, muromonab-CD3, natalizumab, ofatumumab omalizumab, palivizumab, panitumumab, ranibizumab, rituximab, tositumomab, trastuzumab, altumomab pentetate, arcitumomab, atlizumab, bectumomab, belimumab, besilesomab, biciromab, canakinumab, capromab pendetide, catumaxomab, denosumab, edrecolomab, efungumab, ertumaxomab, etaracizumab, fanolesomab, fontolizumab, gemtuzumab ozogamicin, golimumab, igovomab, imciromab, labetuzumab, mepolizumab, motavizumab, nimotuzumab, nofetumomab merpentan, oregovomab, pemtumomab, pertuzumab, rovelizumab, ruplizumab, sulesomab, tacatuzumab tetraxetan, tefibazumab, tocilizumab, ustekinumab, visilizumab, votumumab, zalutumumab, and zanolimumab. The active agents can also be the combinations of any of the antibodies listed above.

As used herein, the term “Complementarity Determining Regions” (CDRs; i.e., CDR1, CDR2, and CDR3) refers to the amino acid residues of an antibody variable domain the presence of which are necessary for antigen binding. Each variable domain typically has three CDR regions identified as CDR1, CDR2 and CDR3. Each complementarity determining region may comprise amino acid residues from a “complementarity determining region” as defined by Kabat (i.e. about residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e. about residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). In some instances, a complementarity determining region can include amino acids from both a CDR region defined according to Kabat and a hypervariable loop.

The expression “linear antibodies” refers to the antibodies described in Zapata et al., Protein Eng., 8(10):1057-1062 (1995). Briefly, these antibodies comprise a pair of tandem Fd segments (VH-CH1-VH-CH1) which, together with complementary light chain polypeptides, form a pair of antigen binding regions. Linear antibodies can be bispecific or monospecific.

The expression “single-chain Fv” or “scFv” antibody fragments, as used herein, is intended to mean antibody fragments that comprise the VH and VL domains of antibody, wherein these domains are present in a single polypeptide chain. Preferably, the Fv polypeptide further comprises a polypeptide linker between the VH and VL domains which enables the scFv to form the desired structure for antigen binding. (The Pharmacology of Monoclonal Antibodies, vol. 113, Rosenburg and Moore eds., Springer-Verlag, New York, pp. 269-315 (1994)).

The term “diabodies,” as used herein, refers to small antibody fragments with two antigen-binding sites, which fragments comprise a heavy-chain variable domain (VH) Connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. (EP 404,097; WO 93/11161; Hollinger et ah, Proc. Natl. Acad. Sd. USA, P0:6444-6448 (1993)).

As used herein, the term “small molecules” refers to natural or synthetic molecules including, but not limited to, peptides, peptidomimetics, amino acids, amino acid analogs, polynucleotides, polynucleotide analogs, aptamers, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

The term “antibiotics” or” antimicrobial compound” is used herein to describe a compound or composition which decreases the viability of a microorganism, or which inhibits the growth or reproduction of a microorganism. As used in this disclosure, an antibiotic is further intended to include an antimicrobial, bacteriostatic, or bactericidal agent. Exemplary antibiotics can include, but are not limited to, actinomycin; aminoglycosides (e.g., neomycin, gentamicin, tobramycin); β-lactamase inhibitors (e.g., clavulanic acid, sulbactam); glycopeptides (e.g., vancomycin, teicoplanin, polymixin); ansamycins; bacitracin; carbacephem; carbapenems; cephalosporins (e.g., cefazolin, cefaclor, cefditoren, ceftobiprole, cefuroxime, cefotaxime, cefipeme, cefadroxil, cefoxitin, cefprozil, cefdinir); gramicidin; isoniazid; linezolid; macrolides (e.g., erythromycin, clarithromycin, azithromycin); mupirocin; penicillins (e.g., amoxicillin, ampicillin, cloxacillin, dicloxacillin, flucloxacillin, oxacillin, piperacillin); oxolinic acid; polypeptides (e.g., bacitracin, polymyxin B); quinolones (e.g., ciprofloxacin, nalidixic acid, enoxacin, gatifloxacin, levaquin, ofloxacin, etc.); sulfonamides (e.g., sulfasalazine, trimethoprim, trimethoprim-sulfamethoxazole (co-trimoxazole), sulfadiazine); tetracyclines (e.g., doxycyline, minocycline, tetracycline, etc.); monobactams such as aztreonam; chloramphenicol; lincomycin; clindamycin; ethambutol; mupirocin; metronidazole; pefloxacin; pyrazinamide; thiamphenicol; rifampicin; thiamphenicl; dapsone; clofazimine; quinupristin; metronidazole; linezolid; isoniazid; piracil; novobiocin; trimethoprim; fosfomycin; fusidic acid; or other topical antibiotics. Optionally, the antibiotic agents may also be antimicrobial peptides such as defensins, magainin and nisin; or lytic bacteriophage. The antibiotic agents can also be the combinations of any of the agents listed above. See also PCT/US2010/026190.

As used herein, the term “antigens” refers to a molecule or a portion of a molecule capable of being bound by a selective binding agent, such as an antibody, and additionally capable of being used in an animal to elicit the production of antibodies capable of binding to an epitope of that antigen. An antigen may have one or more epitopes. The term “antigen” can also refer to a molecule capable of being bound by an antibody or a T cell receptor (TCR) if presented by MHC molecules. The term “antigen”, as used herein, also encompasses T-cell epitopes. An antigen is additionally capable of being recognized by the immune system and/or being capable of inducing a humoral immune response and/or cellular immune response leading to the activation of B- and/or T-lymphocytes. This may, however, require that, at least in certain cases, the antigen contains or is linked to a Th cell epitope and is given in adjuvant. An antigen can have one or more epitopes (B- and T-epitopes). The specific reaction referred to above is meant to indicate that the antigen will preferably react, typically in a highly selective manner, with its corresponding antibody or TCR and not with the multitude of other antibodies or TCRs which may be evoked by other antigens. Antigens as used herein may also be mixtures of several individual antigens.

As used herein, the term “therapeutic agent” generally means a molecule, group of molecules, complex or substance administered to an organism for diagnostic, therapeutic, preventative medical, or veterinary purposes. As used herein, the term “therapeutic agent” includes a “drug” or a “vaccine.” This term include externally and internally administered topical, localized and systemic human and animal pharmaceuticals, treatments, remedies, nutraceuticals, cosmeceuticals, biologicals, devices, diagnostics and contraceptives, including preparations useful in clinical and veterinary screening, prevention, prophylaxis, healing, wellness, detection, imaging, diagnosis, therapy, surgery, monitoring, cosmetics, prosthetics, forensics and the like. This term can also be used in reference to agriceutical, workplace, military, industrial and environmental therapeutics or remedies comprising selected molecules or selected nucleic acid sequences capable of recognizing cellular receptors, membrane receptors, hormone receptors, therapeutic receptors, microbes, viruses or selected targets comprising or capable of contacting plants, animals and/or humans. This term can also specifically include nucleic acids and compounds comprising nucleic acids that produce a bioactive effect, for example deoxyribonucleic acid (DNA), ribonucleic acid (RNA), modified DNA or RNA, or mixtures or combinations thereof, including, for example, DNA nanoplexes.

The term “therapeutic agent” also includes an agent that is capable of providing a local or systemic biological, physiological, or therapeutic effect in the biological system to which it is applied. For example, the therapeutic agent can act to control infection or inflammation, enhance cell growth and tissue regeneration, control tumor growth, act as an analgesic, promote anti-cell attachment, and enhance bone growth, among other functions. Other suitable therapeutic agents can include anti-viral agents, hormones, antibodies, or therapeutic proteins. Other therapeutic agents include prodrugs, which are agents that are not biologically active when administered but, upon administration to a subject are converted to biologically active agents through metabolism or some other mechanism. Additionally, a silk-based composition can contain combinations of two or more therapeutic agents.

In some embodiments, different types of therapeutic agents that can be encapsulated or dispersed in a silk fibroin-based material can include, but not limited to, proteins, peptides, antigens, immunogens, vaccines, antibodies or portions thereof, antibody-like molecules, enzymes, nucleic acids, modified RNA, siRNA, shRNA, aptamers, small molecules, antibiotics, and any combinations thereof.

Exemplary therapeutic agents include, but are not limited to, those found in Harrison's Principles of Internal Medicine, 13^{th} Edition, Eds. T. R. Harrison et al. McGraw-Hill N.Y., NY; Physicians Desk Reference, 50^{th} Edition, 1997, Oradell N.J., Medical Economics Co.; Pharmacological Basis of Therapeutics, 8^{th} Edition, Goodman and Gilman, 1990; United States Pharmacopeia, The National Formulary, USP XII NF XVII, 1990, the complete contents of all of which are incorporated herein by reference.

Embodiments of various aspects described herein can be defined in any of the following numbered paragraphs:
1. A silk fibroin-based composition comprising non-hydrolyzed silk fibroin and a humectant agent.
2. The silk fibroin-based composition of paragraph 1, wherein the non-hydrolyzed silk fibroin has an average molecular weight of at least about 20 kDa or higher.
3. The silk fibroin-based composition of any of paragraphs 1-3, wherein the non-hydrolyzed silk fibroin and humectant agent are present in a volume ratio of about 1:2 to about 1:100.
4. The silk fibroin-based composition of any of paragraphs 1-4, wherein the non-hydrolyzed silk fibroin and humectant agent are present in a volume ratio of about 1:2 to about 1:10.
5. The silk fibroin-based composition of any of paragraphs 1-5, wherein the non-hydrolyzed silk fibroin is present in an amount of about 1% (w/v or w/w) to about 30% (w/v or w/w).
6. The silk fibroin-based composition of any of paragraphs 1-6, wherein the non-hydrolyzed silk fibroin is present in an amount of about 1% (w/v or w/w) to about 10% (w/v or w/w).
7. The silk fibroin-based composition of any of paragraphs 1-7, wherein the humectant agent is selected from the group consisting of phospholipid (e.g., lecithin, panthenol (pro-vitamin B5), glycerin (glycerol)), aloe vera, sorbitol, urea, alpha-hydroxy acids, sodium pyroglutamate, N-acetyl-ethanolamine, sodium lactate, isopropanol, polyalkylene glycols (e.g., ethylene glycol, propylene glycol, hexylene glycol, butylene glycol, dipropylene glycol, triethylene glycol), 1,3-propanediol, polyethylene glycols, synthetic alcohols (e.g., glyceryl coconate, hydroxystearate, myristate, oleate), and any combinations thereof.
8. The silk fibroin-based composition of any of paragraphs 1-8, wherein the humectant agent comprises phospholipid (e.g., lecithin, panthenol (pro-vitamin B5), glycerin), aloe vera, sorbital, alpha-hydroxy acids, or any combinations thereof.
9. The silk fibroin-based composition of any of paragraphs 1-8, wherein the humectant agent comprises glycerin (glycerol).
10. The silk fibroin-based composition of paragraph 9, wherein the glycerin is present in an amount of about 50% (w/v or w/w) to about 99% (w/v or w/w).
11. The silk fibroin-based composition of any of paragraphs 1-10, wherein the composition is a gel or hydrogel, a paste, a lotion, a cream, an ointment, an oil, a liquid, a serum, a shampoo, a foam or mousse, a spray, an aerosol, a scrub, or any combinations thereof.
12. The silk fibroin-based composition of any of paragraphs 1-11, wherein the composition is translucent.
13. The silk fibroin-based composition of any of paragraphs 1-12, wherein the composition is flowable.
14. The silk fibroin-based composition of any of paragraphs 1-13, wherein the composition does not precipitate when exposed to shear.
15. The silk fibroin-based composition of any of paragraphs 1-14, wherein the composition has a larger amount of silk fibroin in random coil conformation, as compared to a composition comprising the non-hydrolyzed silk fibroin in the absence of the humectant agent.
16. The silk fibroin-based composition of any of paragraphs 1-15, wherein the composition has a smaller amount of silk fibroin in beta-sheet conformation, as compared to a composition comprising the non-hydrolyzed silk fibroin in the absence of the humectant agent.
17. The silk fibroin-based composition of any of paragraphs 1-16, wherein the composition does not have a peak in the amide II region (between ˜1475 cm⁻¹ and ˜1560 cm⁻¹), as determined in a Fourier Transform Infrared Spectroscopy (FTIR) spectrum.
18. The silk fibroin-based composition of any of paragraphs 1-17, further comprising an additive.
19. The silk fibroin-based composition of paragraph 18, wherein the additive comprises aqueous-soluble components, oil-soluble components, wax-soluble components, essential oils, emulsifiers, surfactants, flavors, fragrance, nutraceuticals, vitamins, therapeutic agents, cosmeceuticals, preservatives, botanical extracts or distillates, antioxidants, anti-bacterial agents, anti-inflammatory agents, cosmetically-acceptable agents, skin-lightening agents, skin-evening agents, anti-reddening agents, viscosity modifiers, emollients, or any combinations thereof.
20. The silk fibroin-based composition of any of paragraphs 1-19, wherein the composition is formulated for use in a personal care product (e.g., a cosmetic, skincare, body care, or hair product).
21. The silk fibroin-based composition of any of paragraphs 1-20, wherein the composition is formulated for use in a food product.
22. The silk fibroin-based composition of any of paragraphs 1-21, wherein the composition is stable (e.g., no brittle gel formation) for at least about 1 month at room temperature.
23. A composition comprising an aqueous-based phase, an oil-based phase or a wax-based phase, and an emulsifier, wherein the aqueous-based phase comprises non-hydrolyzed silk fibroin and a humectant agent.
24. The composition of paragraph 23, wherein the non-hydrolyzed silk fibroin has an average molecular weight of at least about 20 kDa or higher.
25. The composition of any of paragraphs 23-24, wherein the non-hydrolyzed silk fibroin and humectant agent are present in a volume ratio of about 1:2 to about 1:100 in the aqueous-based phase.
26. The composition of any of paragraphs 23-25, wherein the non-hydrolyzed silk fibroin and humectant agent are present in a volume ratio of about 1:2 to about 1:10 in the aqueous-based phase.
27. The composition of any of paragraphs 23-26, wherein the non-hydrolyzed silk fibroin is present in an amount of about 1% (w/v or w/w) to about 30% (w/v or w/w) of the aqueous phase.
28. The composition of any of paragraphs 23-27, wherein the non-hydrolyzed silk fibroin is present in an amount of about 1% (w/v or w/w) to about 10% (w/v or w/w) of the aqueous phase.
29. The composition of any of paragraphs 23-28, wherein the humectant agent is selected from the group consisting of phospholipid (e.g., lecithin, panthenol (pro-vitamin B5), glycerin (glycerol)), aloe vera, sorbitol, urea, alpha-hydroxy acids, sodium pyroglutamate, N-acetyl-ethanolamine, sodium lactate, isopropanol, polyalkylene glycols (e.g., ethylene glycol, propylene glycol, hexylene glycol, butylene glycol, dipropylene glycol, triethylene glycol), 1,3-propanediol, polyethylene glycols, synthetic alcohols (e.g., glyceryl coconate, hydroxystearate, myristate, oleate), and any combinations thereof.
30. The composition of any of paragraphs 23-29, wherein the humectant agent comprises phospholipid (e.g., lecithin, panthenol (pro-vitamin B5), glycerin), aloe vera, sorbital, alpha-hydroxy acids, or any combinations thereof.
31. The composition of any of paragraphs 23-28, wherein the humectant agent comprises glycerin (glycerol).
32. The composition of paragraph 31, wherein the humectant agent is present in an amount of about 50% (w/v or w/w) to about 99% (w/v or w/w) in the aqueous-based phase.
33. The composition of any of paragraphs 23-32, wherein the oil-based phase comprises a carrier oil.
34. The composition of paragraph 33, wherein the carrier oil is a synthetic or natural oil derived from seeds, nuts, fruits, flowers, plant-based materials, or any combinations thereof.
35. The composition of any of paragraphs 23-32, wherein the wax-based phase comprises butters, wax, paraffin wax, paraffin oil, petrolatum, or any combinations thereof.
36. The composition of any of paragraphs 23-35, wherein at least one or both of the aqueous-based phase and the oil-based phase or the wax-based phase further comprise an additive.
37. The composition of paragraph 36, wherein the additive comprises aqueous-soluble components, oil-soluble components, wax-soluble components, essential oils, surfactants, flavors, fragrance, nutraceuticals, vitamins, therapeutic agents, preservatives, botanical extracts or distillates, antioxidants, anti-bacterial agents, anti-inflammatory agents, cosmetically-acceptable agents, cosmeceuticals, skin-lightening agents, skin-evening agents, anti-reddening agents, viscosity modifiers, emollients, or any combinations thereof.
38. The composition of any of paragraphs 23-37, wherein the ratio of the aqueous-based phase to the oil-based phase is about 1:100 to about 100:1.
39. The composition of any of paragraphs 23-38, wherein the emulsifier is selected from the group consisting of liquid soy lecithin, solid soy lecithin, honey, beeswax, cetyl alcohol, or any combinations thereof.
40. The composition of any of paragraphs 23-39, wherein the composition is a gel or hydrogel, a paste, a lotion, a cream, an ointment, an oil, a liquid, a serum, a shampoo, a foam or mousse, a spray, an aerosol, a stick, a balm, a bar, a scrub, or any combinations thereof.
41. The composition of any of paragraphs 23-40, wherein the composition is translucent.
42. The composition of any of paragraphs 23-41, wherein the composition is flowable.
43. The composition of any of paragraphs 23-42, wherein the composition does not precipitate when exposed to shear.
44. The composition of any of paragraphs 23-43, wherein the composition is formulated for use in a personal care product (e.g., a cosmetic, skincare, body care, or hair product).
45. The composition of any of paragraphs 23-44, wherein the composition is formulated for use in a food product.
46. A method of producing a silk fibroin-based composition comprising: mixing a non-hydrolyzed silk fibroin solution and a humectant agent in a volume ratio of about 1:2 to about 1:100.
47. The method of paragraph 46, wherein the volume ratio of the non-hydrolyzed silk fibroin solution to the humectant agent is about 1:2 to about 1:10.
48. The method of paragraph 46 or 47, wherein the humectant agent is selected from the group consisting of phospholipid (e.g., lecithin, panthenol (pro-vitamin B5), glycerin (glycerol)), aloe vera, sorbitol, urea, alpha-hydroxy acids, sodium pyroglutamate, N-acetyl-ethanolamine, sodium lactate, isopropanol, polyalkylene glycols (e.g., ethylene glycol, propylene glycol, hexylene glycol, butylene glycol, dipropylene glycol, triethylene glycol), 1,3-propanediol, polyethylene glycols, synthetic alcohols (e.g., glyceryl coconate, hydroxystearate, myristate, oleate), and any combinations thereof.
49. The method of any of paragraphs 46-48, wherein the humectant agent comprises phospholipid (e.g., lecithin, panthenol (pro-vitamin B5), glycerin), aloe vera, sorbital, alpha-hydroxy acids, or any combinations thereof.
50. The method of any of paragraphs 46-49, wherein the non-hydrolyzed silk fibroin solution has a concentration of about 1% (w/v or w/w) to about 30% (w/v or w/w).
51. The method of any of paragraphs 46-50, wherein the non-hydrolyzed silk fibroin solution has a concentration of about 1% (w/v or w/w) to about 10% (w/v or w/w).
52. The method of any of paragraphs 46-51, further comprising mixing an additive with the humectant agent and the non-hydrolyzed silk fibroin solution.
53. The method of paragraph 52, wherein the additive is mixed with the humectant agent prior to said mixing with the non-hydrolyzed silk fibroin solution.
54. The method of any of paragraphs 46-53, further comprising mixing an aqueous mixture comprising the non-hydrolyzed silk fibroin solution and the humectant agent with an oil-based phase or a wax-based phase.
55. The method of paragraph 54, wherein the oil-based phase comprises a carrier oil and an emulsifier.
56. The method of paragraph 55, wherein the carrier oil is a synthetic or natural oil derived from seeds, nuts, fruits, flowers, plant-based materials, or any combinations thereof.
57. The method of paragraph 54, wherein the wax-based phase comprises butters, wax, paraffin wax, paraffin oil, petrolatum, or any combinations thereof.
58. The method of any of paragraphs 55-57, wherein the emulsifier is selected from the group consisting of liquid soy lecithin, solid soy lecithin, honey, beeswax, cetyl alcohol, or any combinations thereof.
59. The method of any of paragraphs 55-58, wherein the volume ratio of the carrier oil to the emulsifier is about 2:1 to about 100:1.
60. The method of any of paragraphs 54-59, wherein the oil-based phase further comprises an additive.
61. The method of paragraph 52 or 60, wherein the aqueous-soluble or the oil-soluble additive is selected from the group consisting of essential oils, flavors, fragrance, nutraceuticals, therapeutic agents, preservatives, botanical extracts or distillates, antioxidants, anti-bacterial agents, anti-inflammatory agents, cosmetically-acceptable agents, viscosity modifiers, emollients, or any combinations thereof.
62. A composition produced by the method of any of paragraphs 46-61.
63. The composition of paragraph 62, wherein the composition is formulated for use in a personal care product (e.g., a cosmetic, skincare, body care, or hair product).
64. The composition of paragraph 62, wherein the composition is formulated for use in a food product.
65. A method comprising:
   applying on a target surface a composition of any of paragraphs 1-45 or 62-64.
66. The method of paragraph 65, wherein the applying comprises rubbing.
67. The method of paragraph 65 or 66, wherein the target surface is skin or hair of a subject.
68. The method of any of paragraphs 65-67, further comprising maintaining the composition at room temperature for at least about 1 month, wherein the composition remains stable thereafter.

### SOME ELECTED DEFINITIONS

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

As used herein the term “comprising” or “comprises” is used in reference to compositions, methods, and respective component(s) thereof, that are essential to the invention, yet open to the inclusion of unspecified elements, whether essential or not.

The singular terms “a,” “an,” and “the” include plural referents unless context clearly indicates otherwise. Similarly, the word “or” is intended to include “and” unless the context clearly indicates otherwise.

Other than in the operating examples, or where otherwise indicated, all numbers expressing quantities of ingredients or reaction conditions used herein should be understood as modified in all instances by the term “about.” The term “about” when used in connection with percentages may mean±5% of the value being referred to. For example, about 100 means from 95 to 105.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

The term “statistically significant” or “significantly” refers to statistical significance and generally means at least two standard deviation (2SD) away from a reference level. The term refers to statistical evidence that there is a difference. It is defined as the probability of making a decision to reject the null hypothesis when the null hypothesis is actually true.

As used interchangeably herein, the term “substantially” means a proportion of at least about 60%, or preferably at least about 70% or at least about 80%, or at least about 90%, at least about 95%, at least about 97% or at least about 99% or more, or any integer between 70% and 100%. In some embodiments, the term “substantially” means a proportion of at least about 90%, at least about 95%, at least about 98%, at least about 99% or more, or any integer between 90% and 100%. In some embodiments, the term “substantially” can include 100%.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated may be further modified to incorporate features shown in any of the other embodiments disclosed herein.

The disclosure is further illustrated by the following examples which should not be construed as limiting. The examples are illustrative only, and are not intended to limit, in any manner, any of the aspects described herein. The following examples do not in any way limit the invention.

### EXAMPLES

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### Example 1

### Stabilization of Non-Hydrolyzed Silk Fibroin Protein for Use in Skin and Hair Products

In order to address an unmet need for all-natural beauty products, inventors discovered methods of using non-hydrolyzed silk protein as a component for formulating 100% all-natural beauty products. Silk fibers are commonly used in the textile industry for their luster, strength, and lightweight quality. These qualities are also desirable for skin and hair. Unfortunately, non-hydrolyzed silk protein has (1) a short shelf life before spontaneous gelation occurs and (2) the material precipitates when rubbed onto skin and hair. Both of these material properties are undesirable for formulating skin and hair care products. To overcome these challenges, the inventors developed a novel technology for stabilizing non-hydrolyzed silk fibroin protein so that non-hydrolyzed silk fibroin protein can be incorporated into and provide the aesthetic quality of beauty products that are 100% all-natural. These products can offer consumers or users effective, long-lasting moisture and protection for skin and/or hair with a 100% all-natural formula that has a pleasant aesthetic.

Below are exemplary materials and methods that can be used to make beauty product compositions comprising non-hydrolyzed silk fibroin proteins. It will be apparent to those skilled in the relevant art that various modifications, additions, substitutions, and the like can be made without departing from the spirit of various aspects described herein and these are therefore considered to be within the scope of various aspects described herein. Methods

### Exemplary Materials and Methods

Non-Hydrolyzed Silk Fibroin Solution.

Bombyx mori silkworm cocoons were supplied by Tajimia Shoji Co. (Yokohama, Japan). Briefly, cocoons were cut and placed in boiling ˜0.02 M Na₂CO₃ for about 10-60 minutes to remove sericin, then washed three times in ultrapure water. The resulting silk fibroin fibers were dried overnight. The dry silk fibers were solubilized in ˜9.3 M lithium bromide (LiBr) at 20% w/v for about four hours at ˜60° C. The silk solution was dialyzed against ultrapure water with a 3,500 MWCO membrane for about two days, with a total of about six water changes, to remove the LiBr. The resulting silk fibroin solution concentration was about 6-8% silk in water (w/v). In some embodiments, the silk fibroin solution can be concentrated to about 20% (w/v), e.g., using centrifugal filters with about 3500 MWCO.

Silk Protein Stabilization and Beauty Product Formulation.

In some embodiments, the beauty product formulation platform comprises two phases: phase A and phase B. Phase A comprises aqueous-based ingredients and phase B comprises oil-based ingredients. The two phases were brought together using natural emulsifiers. Phase A and phase B were prepared separately and then gently mixed together for an optimum time, e.g., approximately two to five minutes, after which point, the mixture began to gel. It should be noted that mixing too hard can cause the silk to congeal but phase separation can occur when mixing is not sufficient. Examples of the various ingredients that can be included the formulation are listed in Table 1. It should be noted that silk protein stabilization and beauty product formulation can be achieved with a wide range and various combinations of aqueous protein solutions, all-natural or synthetic humectants, all-natural or synthetic emulsifiers, and/or all-natural or synthetic oils and thus are not limited to the ingredients listed in Table 1.

TABLE 1
Example ingredients that can be used in formulating all-natural,
silk-based beauty products

| Phase | Example ingredients |
|---|---|
| Phase A—aqueous-based | non-hydrolyzed silk fibroin protein |
| components | humectant (e.g., glycerin, aloe vera) |
| | botanical distillates |
| | aqueous-soluble vitamins and/or |
| | minerals |
| Phase B—oil/wax-based | carrier oils |
| components | butters |
| | essential oils |
| | oil-soluble vitamins/minerals |
| Emulsifiers | liquid soy lecithin |
| | solid soy lecithin |
| | honey |
| | beeswax |
| | cetyl alcohol |

For phase A, a humectant (e.g., glycerin) and all other aqueous-based components except the non-hydrolyzed silk fibroin were mixed together. Next, non-hydrolyzed silk fibroin protein solutions with concentration ranges of ˜5-8% w/v were combined with the glycerin mixture in a volume ratio of non-hydrolyzed silk fibroin to glycerin ranging from 1:8 to 1:2. These two components were mixed continually, e.g., for approximately five minutes, before adding phase B. For phase B, the emulsifier, carrier oils and essential oils were mixed until fully combined. Phase B was then added to phase A with constant stirring, e.g., for approximately five minutes.

Assessment of Non-Hydrolyzed Silk Fibroin Protein and Silk-Based Formulations.

Fourier transform infrared spectroscopy (FTIR) was used to assess the β-sheet content and intermolecular interactions of the silk samples. Samples were cast on Teflon-coated polystyrene dishes and air dried for 12 h. FTIR measurements were performed with a Bruker Equinox 55/S FTIR spectrometer (Bruker Optics, Billerica, Mass.) and measurements were taken with 132 scans ranging from 400 to 4000 cm⁻¹ at 4 cm⁻¹ resolution. The spectra were fourier self-deconvoluted (FSD) with Opus 5.5 spectroscopy software (Bruker Optics) using the method reported in Hu et al., 2006. To determine β-sheet content, the FSD FTIR spectra were fitted with Gaussian profiles in the amide I region between 1595 and 1705 cm⁻¹.

SDS-PAGE was used to compare the electrophoretic mobility of non-hydrolyzed silk fibroin protein and hydrolyzed silk protein. The protein was loaded at two different concentrations, “High”=˜35 μg and “Low”=˜5 μg.

For fluorescence, silk solutions were diluted to ˜0.1% w/v and excited with a ˜280 nm laser. The emission spectra were collected between ˜290 and ˜400 nm.

Hair Treated with ALLSilk™ (a Trademark for a Composition Comprising Non-Hydrolyzed Silk Fibroin and a Humectant).

The effect of the ALLSilk™ hair serum (an example formulation is shown in Table 2 below) on hair cuticle smoothness was evaluated by daily application of the hair serum to half a head of hair for about 80 days. The other side of the head was left untreated. Scanning electron microscopy (SEM) was performed on hair samples taken at day 0, 7, 28, 60 and 82. Hair strands were sputter coated with platinum/palladium prior to SEM imaging.

### Results

### Product Physical Properties

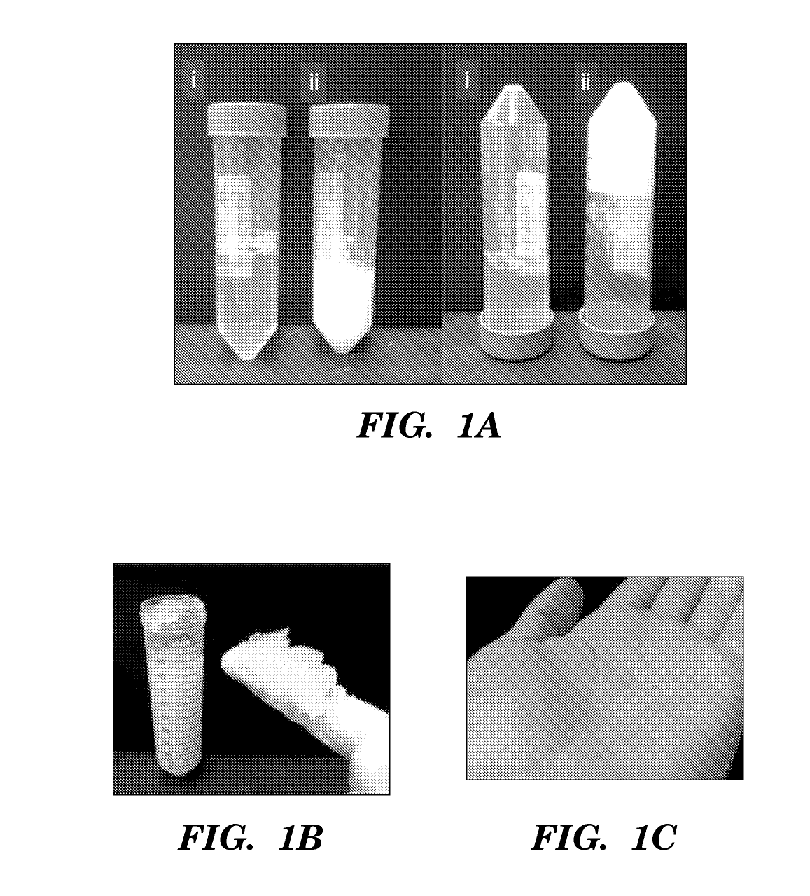

Various embodiments described herein relate to all-natural beauty products with desirable aesthetic qualities while delivering a shelf-stable product. Non-hydrolyzed silk fibroin protein solution is a liquid (hereafter referred to as “liquid silk”) for up to about 4-8 weeks at 4° C. (FIG. 1A, panel i), but spontaneously forms a brittle gel thereafter (FIG. 1A, panel ii and FIG. 1B). This spontaneously formed gel is considered “expired” because it can no longer be manipulated (hereafter referred to as “expired silk”). Furthermore, when liquid silk is rubbed on the skin, the protein material precipitates and forms “pills” on the skin surface (FIG. 1C). These two qualities of non-hydrolyzed silk fibroin protein make it undesirable for use alone as a beauty product.

To overcome these challenges of using non-hydrolyzed silk fibroin protein in beauty products, a platform technology was developed, in part, for (1) stabilizing non-hydrolyzed silk fibroin protein at room temperature or above for >12 months, and (2) enabling the non-hydrolyzed silk fibroin protein to be sheared on the skin without precipitation of the protein. This technology involved the coordinated combination of non-hydrolyzed silk fibroin protein with a humectant, e.g., an all-natural humectant.

In one embodiment, the all-natural humectant to be used in combination with non-hydrolyzed silk fibroin protein was glycerin. The resulting product or mixture had a gel-like consistency that was translucent and flowable (hereafter referred interchangeably to as “stabilized silk gel” or “ALLSilk™ gel”), distinctly different from the expired silk. When the stabilized silk gel was rubbed on the skin, a precipitate was not observed. It should be noted that a similar observation was made upon mixing non-hydrolyzed silk fibroin protein with aloe vera (data not shown).

### Non-Hydrolyzed Silk Fibroin Protein Stabilization Mechanism

Non-hydrolyzed silk fibroin protein (liquid silk) exhibits a characteristic FTIR spectrum in the amide I and amide II region, which lacks a peak in the 1600-1640 cm⁻¹ β-sheet absorbance region and exhibits a minimal peak in the tyrosine side-chain absorbance region (FIG. 2A, “non-hydrolyzed silk fibroin protein”). Non-hydrolyzed silk fibroin protein primarily exists in the random coil formation. When non-hydrolyzed silk fibroin protein shelf life expires, it spontaneously gels and becomes expired silk. Without wishing to be bound by theory, the gelling mechanism can be explained, in part, by a shift from the random-coil formation to the β-sheet formation. FTIR spectra demonstrated that the expired silk contains a prominent β-sheet peak in the amide I region and a prominent peak in the tyrosine side-chain region in amide II (FIG. 2A, “expired silk”). High β-sheet content is a hallmark of silk protein that has formed extensive intermolecular hydrogen bonds and thereafter insoluble in aqueous solutions. The increase in the tyrosine side-chain peak is indicative of the dense packing of the silk molecule when β-sheets are formed. In contrast, the FTIR spectra of the non-hydrolyzed silk fibroin protein stabilized with glycerin had minimal β-sheet content (FIG. 2A, “ALLSilk™ gel”). The differences detected in the ALLSilk™ gel are derived, at least in part, from the non-hydrolyzed silk fibroin-glycerin interaction and not from the glycerin alone, which lacks a peak in the amide I region (FIG. 2A, “glycerin”). Surprisingly, the stabilized silk gel was also devoid of a peak in the amide II region, which was distinctly different from the expired silk or the insoluble silk. Thus, the mechanism of the ALLSilk™ gel technology is distinctly different from other mechanisms of silk gelation, which primarily involve β-sheet formation. In contrast, the ALLSilk™ technology exhibits silk gelation without or with minimal β-sheet formation and maintains dominant random-coil content. Without wishing to be bound by theory, the difference in the silk fibroin conformation (random-coil vs. beta-sheet) determined in the ALLSilk™ gel and expired gel likely contribute, in part, to the flow-ability of the ALLSilk™ gel.

It has been previously reported that silk gelation can be controllably induced by lowering the pH of the silk solution (Yucel et al., 2010). To determine if the gelation mechanism of the stabilized gel was different from pH-induced gelation, glycerin in the solution comprising non-hydrolyzed silk fibroin and the glycerin was replaced with water, and the pH of the solution was then reduced to pH ˜1, ˜3, and ˜5 (FIG. 2B). In the absence of glycerin, the silk precipitated only at the pH 1 condition. However, when silk is combined with glycerin of pH of ˜5, ˜7, and ˜10, a stabilized silk gel was formed regardless of the pH (FIG. 2C). In addition, the pH affected the kinetics of gelation, e.g., lowering the pH decreased the time to gelation.

### Hair and Skin Serum Formulation

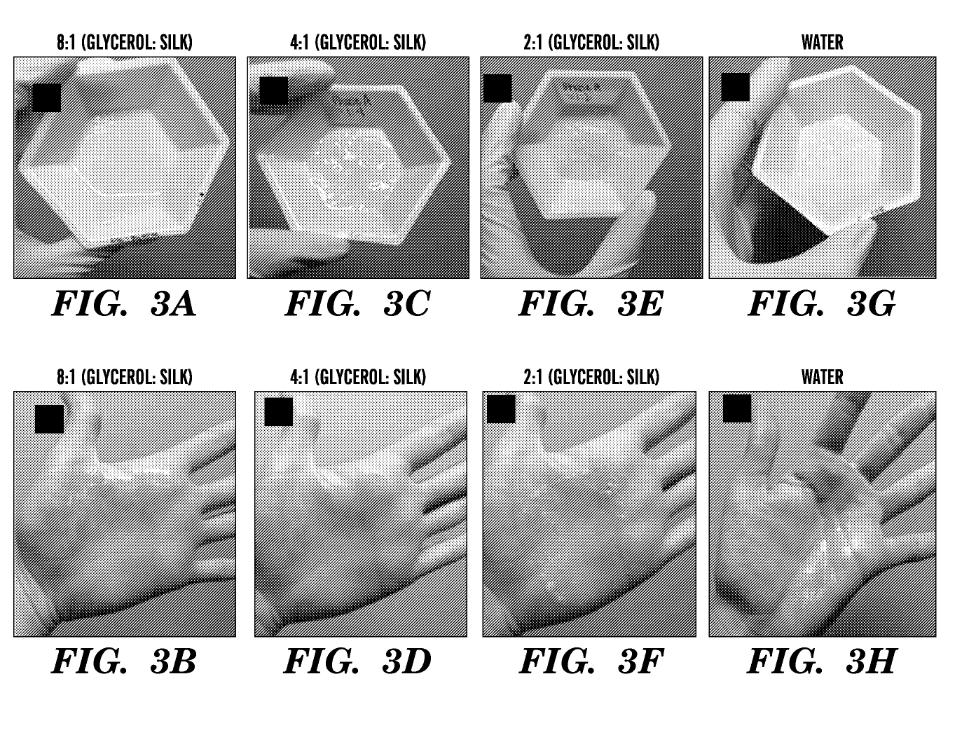

To assess the ability of the stabilized silk gel to be used in a beauty product formulation, in one embodiment, a serum was made. Beauty products typically comprise an oil phase and an aqueous phase (e.g., a water phase) with an emulsifier to combine the two phases. In one embodiment, an optimized formulation listed in Table 2 below was evaluated. In this example, the ratio of the aqueous-based phase to the oil-based phase is about 5:1 to about 15:1 (e.g., about 9:1) to form the serum. The stabilized silk gel played a pivotal role in formulating an emulsified, aesthetically pleasing serum material for skin and hair (FIGS. 3A-3H). To give the serum an aesthetic feel of currently-existing beauty products, different ratios of glycerol to silk solution were evaluated. By increasing the ratio of glycerin to silk, a more flowable gel was formed, but the oil phase and the aqueous phase did not phase separate in any of the samples (FIGS. 3A-3F). When the non-hydrolyzed silk fibroin was replaced with water, there was clear phase separation between the oil phase and the aqueous phase and the mixture did not form a gel and no effects were noted with shear (FIGS. 3G-3H). Thus, even with an emulsifier in the formulation, the non-hydrolyzed silk fibroin protein also played a role in emulsification of the oil and water phases.

TABLE 2
Optimized serum formulation

| Phase | Components |
|---|---|
| A—aqueous phase | 4 parts humectant (e.g., glycerol): 1 part 7% non- |
| | hydrolyzed silk fibroin solution (v/v) |
| | 2% kiwi distillate |
| | (Add silk to glycerin/kiwi mixture) |
| B—oil phase + | essential oils to lecithin - 2:1 (v/v) |
| emulsifier | essential oil mixture- lavender:jojoba:vitamin |
| | E - 1:6:4 (v/v) |
| | (Add essential oils to lecithin) |

Components in order of highest proportion: water, a humectant (e.g., glycerol), non-hydrolyzed silk protein, an emulsifier (e.g., soy lecithin), carrier oil (e.g., jojoba oil and Vitamin E oil (e.g., derived from safflower seed), and favors and fragrance (e.g., kiwi distillate, essential oils such as lavender essential oil).

### Molecular Weight Differences in Hydrolyzed Versus Intact Silk Protein

While some of the existing beauty products contain silk, the silk component that is used in those beauty products is in the hydrolyzed form of the protein. To compare hydrolyzed silk and non-hydrolyzed silk fibroin protein, the electrophoretic mobility of the hydrolyzed and non-hydrolyzed silk protein solutions was assessed using SDS-PAGE gels (FIG. 4A). No protein band from the hydrolyzed silk sample was detected on the SDS-PAGE gel, indicating that the hydrolyzed silk has a molecular weight that is less than the lowest marker, i.e., ˜30 kDa or lower, while non-hydrolyzed silk sample or intact silk fibroin was determined to have a range of molecular weights from less than 30 kDa up to 460 kDa (FIG. 4A). The range of molecular weights for intact silk fibroin (or non-hydrolyzed silk sample) can be controlled with boiling time (minutes of boiling—abbreviated as “mb”) during degumming. Increasing boiling time from about 10 to 60 minutes generally decreases the molecular weight range.

The fluorescent emission spectrum of hydrolyzed and non-hydrolyzed silk protein solutions offers insight into residue side chain interactions. FIG. 4B shows that the hydrolyzed silk solution has a different emission spectra compared to 30 mb and 60 mb non-hydrolyzed silk solution, further indicating that hydrolyzed silk and non-hydrolyzed silk fibroin proteins are distinctly different materials. Further, when the non-hydrolyzed silk fibroin protein component was replaced with hydrolyzed silk at a ratio of 4:1 glycerol to silk, the resulting hydrolyzed silk-glycerol mixture did not exhibit gelation (FIG. 4C).

Improved Cuticle Surface after Use of ALLSilk™ Serum

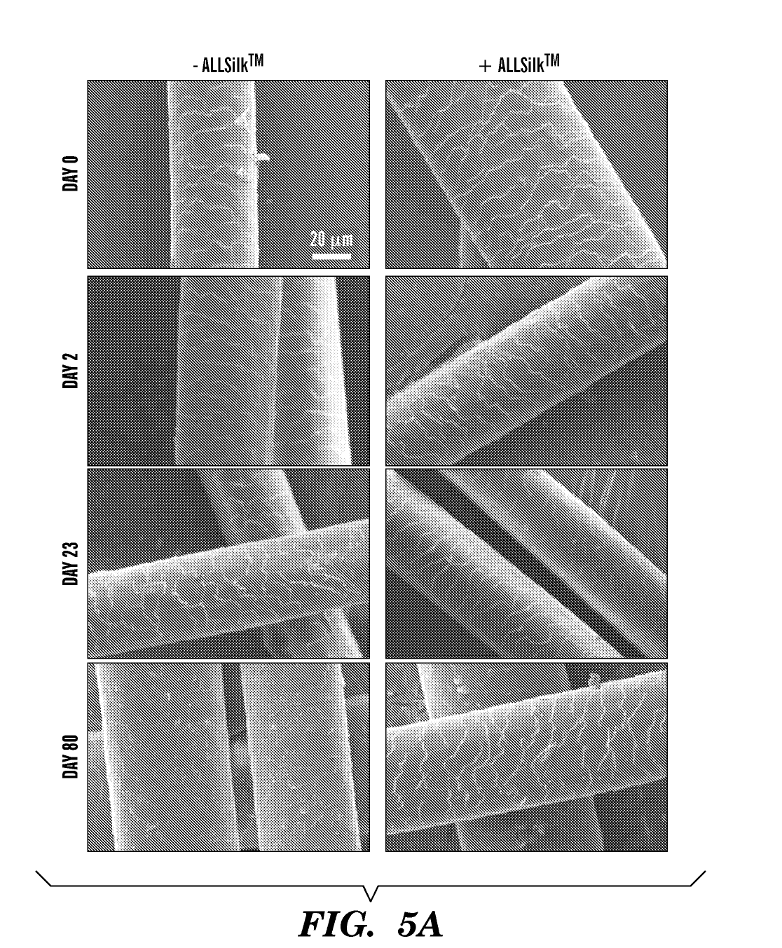

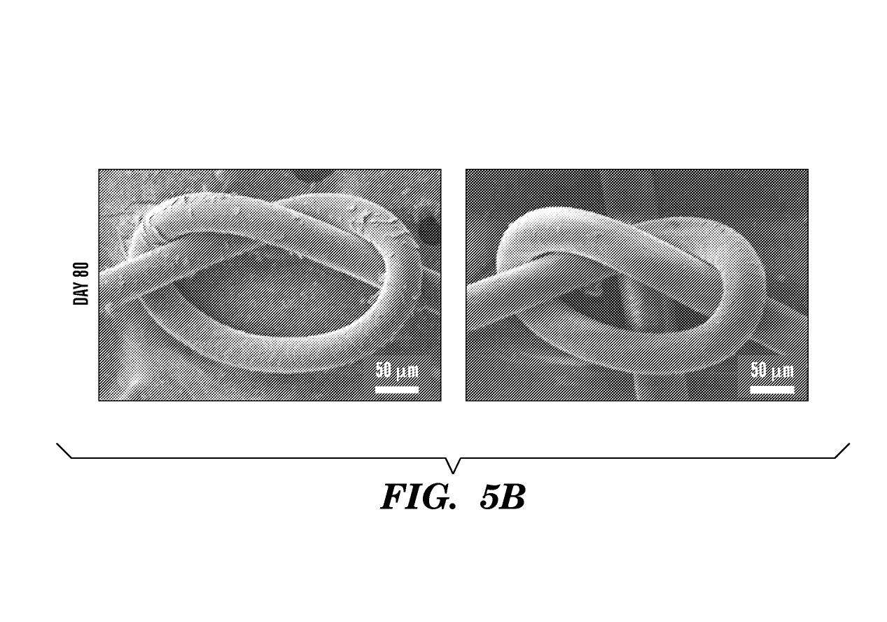

Samples of hair treated with or without ALLSilk™ serum formulation (e.g., a serum formulation as listed in Table 2) were assessed by SEM imaging. Over the course of about 80 days, untreated hair exhibited cuticles that became increasingly more rough (FIG. 5A). This was further illustrated by hair strands that were tied into a pretzel shape. At day 80, a noticeable difference in the cuticle smoothness was observed between the untreated and treated hair (FIG. 5B).

### Discussion

The need for all-natural products is becoming increasingly important as some of the synthetic components are shown to be dangerous to human health and/or to the environment, e.g., in the long term. However, natural products typically do not match the aesthetic quality of synthetic products. Presented in this example are exemplary methods and compositions that can be used to formulate all-natural, shelf-stable beauty products with an aesthetic quality comparable to synthetic analogs. This novel method and composition involves the use of a stabilized gel comprising non-hydrolyzed silk fibroin protein and a humectant (e.g., glycerin).

Silk protein gels have been previously formed using various methods, including sonication, vortexing (shear), electrical stimulation, pH, and any combinations thereof. Without wishing to be bound by theory, sonication and vortexing (shear) rely on physical force to produce local nucleation sites that self-assemble into clusters and eventually crosslinks and entanglements. On the other hand, both electrical stimulation- and pH-formed gels use changes in hydrogen ions or pH differences to form the gel. Silk can also form a gel using lyophilization, which involves the forcible removal of water. As water is removed, the hydrophobic regions of silk are organized into β-sheet structures, stabilizing the material to form the gel.

The stabilized silk gel presented herein involves the coordinated combination of non-hydrolyzed silk fibroin protein and a humectant (e.g., glycerin). Glycerin or glycerol is a strong humectant, where at 70% glycerol solution, 100% of water is bound (Dikstein et al., 1995). Glycerol also stabilizes proteins (Vagenende et al 2009). Without wishing to be bound by theory, it is likely that the addition of glycerol to silk stabilizes the random coil silk structure while attracting water away from hydrogen bonding sites that weakly coordinate, resulting in a flowable gel material. Unlike other gelation methods that produce silk gels that are more brittle (e.g., sonication-induced silk gel), non-flowing (e.g., sonication-induced silk gel), and/or shear-thickening (e.g., electrogelation silk gel, and pH-induced silk gel), the methods and compositions described herein produce a silk fibroin-based gel that can be flowed and sheared without precipitation.

One of the advantages of this ALLSilk™ technology is that it is highly amenable to a variety of beauty product formulations and other applications where a flowable gel material is desirable. In one embodiment, the composition can be adapted to form a serum gel (e.g., with a formulation shown in Table 2) for use as a hair treatment. However, by altering the components of the formulation, the composition can be tailored to a variety of different applications, e.g., skin and/or hair treatment, e.g., by incorporating into the formulation appropriate components, e.g., but not limited to, botanical extracts, moisturizing oils, fragrance, preservatives, and any combinations thereof.

To form stable water-in-oil emulsions, an emulsifier is necessary. In this example, the aqueous-based phase comprising non-hydrolyzed silk fibroin protein can be incorporated into a beauty product formulation comprising an oil-based phase with an emulsifier, e.g., liquid soy lecithin, but other emulsifiers including natural emulsifiers such as beeswax and honey, can also be used. Moisturizing oils, also referred to as emollients, aid in moisturizing by helping formation of a barrier on the skin or hair (Thiele et al., 2005). For example, one embodiment of the formulation described herein comprises natural emollients, e.g., but not limited to, the Vitamin E and Jojoba oil ingredients. Botanical extracts are commonly included in beauty products for their antioxidant, anti-bacterial and/or anti-inflammatory qualities, for example. Additionally or alternatively, one embodiment of the formulation described comprises botanical extracts, e.g., but not limited to, kiwi distillate, which can protect against oxidative DNA damage (Collins et al., 2001). Fragrance also plays a role in the aesthetic of a beauty product, however many fragrances have been found to be harmful to human health. An alternative to synthetic fragrances is all-natural essential oils. Essential oils are generally derived from plants and have been widely used in aromatherapy. In one embodiment, the formulation described herein can comprise lavender essential oil, which aside from having a pleasant scent, can have many health benefits (Cavanagh et al., 2002). By mixing and matching different essential oils, a variety of fragrances can be developed that can also be tailored to have the desired health benefits appropriate to the specific beauty product application.

Non-hydrolyzed silk fibroin protein has many desirable qualities for skin and hair, e.g., strength, softness, luster, and lightweight feel. However, non-hydrolyzed silk fibroin protein alone has a low shelf-life and shear-thickens when rubbed on the skin. The inventors have discovered inter alia a method for stabilizing non-hydrolyzed silk fibroin protein for an extended shelf life and resistance to shear-thickening. In some embodiments, the stabilized non-hydrolyzed silk fibroin gel can be incorporated into beauty product formulations. Further, as shown in this example, the non-hydrolyzed silk fibroin protein can play a pivotal role in maintaining the oil-water emulsion in the beauty product formulation. The non-hydrolyzed silk-based beauty product formulations are versatile because various aqueous-based and oil-based ingredients can be changed to tailor the product to a desired use on skin and/or hair, and evan in other applications, e.g., food applications.

### Example 2

### Various Examples of Compositions Comprising Non-Hydrolyzed Silk Fibroin Adapted for Use as Different Skincare and Body Care Products

In the following example compositions, a non-hydrolyzed silk fibroin solution is prepared as described in Example 1. The compositions described in this Example generally have a cream-like or scrub texture.

Face Scrub (Get Up and go Java Scrub).

An example of a face scrub formulation is shown in Table 3. Dry ingredients (salt, coffee grounds, buttermilk powder) were combined separately. The remaining wet ingredients were then combined. Dry and wet ingredients were combined and mixed thoroughly, and then stored at ˜4° C.

TABLE 3
An exemplary formulation of a face scrub

| Ingredient | Amount |
|---|---|
| Salt | 1 tsp |
| Course coffee grounds | 1 tsp |
| Fine coffee grounds | 1 tsp |
| Butter milk powder | 4 tsp |
| Oatmeal mixture (1:1 oatmeal powder:boiling | 1 tsp |
| water) | |
| Non-hydrolyzed silk fibroin | 1 tsp |
| Honey | 1 tsp |
| Egg whites | ½ tsp |
| Shea butter | 1 tsp |
| Greek yogurt | 1 tsp |

The face scrub can be applied to the face after moistening the skin and can be massaged into the skin for several seconds in a circular motion. The scrub is then washed off. For added benefits, the scrub can be left on for several minutes before washing off. The face scrub contains exfoliants (e.g., coffee grounds, salt, oats) that when rubbed on the face can slough off the outer layers of the skin and open up the pores. After the scrub is washed off, the silk component leaves the face feeling refreshed and looking lustrous.

Body Butter (Personal Protection Body Butter).

An example of a body butter formulation is shown in Table 4. In a double boiler, shea butter, coconut oil and beeswax were combined. When all the ingredients melted together, the mixture was removed from heat, and essential oils and non-hydrolyzed silk fibroin was added immediately. The solution was then whipped immediately with an electric mixer until thoroughly mixed. Let cool and store at ˜4° C.

TABLE 4
An exemplary formulation of a body butter

| Ingredient | Amt | |
|---|---|---|
| Shea butter | 2 | tbs |
| Coconut oil | 8 | tbs |
| Beeswax | 2 | tbs |
| Lemon essential oil | 20 | drops |
| Geranium (10x dilution in grapeseed oil) | 8 | drops |
| Non-hydrolyzed silk fibroin | 2 | tbs |

The body butter is a thick cream that spreads easily on skin. The skin feels soft and appears lustrous after application.

Foot Cream (Closed-Toe Shoe Foot Treatment).

An example of a foot cream formulation is shown in Table 5. All ingredients were combined and mixed thoroughly, and then stored at ˜4° C.

TABLE 5
An exemplary formulation of a foot cream

| Ingredient | Amt |
|---|---|
| Shea butter | 1 tsp |
| Tea tree oil | Few drops |
| Lemon | Same as tea tree |
| Clove | Dash |
| Ginger | Dash |
| Non-hydrolyzed silk fibroin | 1 tsp |

The foot cream is thick paste that spreads easily onto the feet. The feet are left moisturized, lustrous and not greasy.

Hand Cream (Lab Glove Repair Hand Cream).

An example of a hand cream formulation is shown in Table 6. In a double boiler the water and soy lecithin were combined. The soy lecithin was allowed to melt before adding the shea butter and sweet almond oil. The mixture was then mixed thoroughly. When all ingredients were combined, the mixture was removed from heat, and the essential oil, extract, silk, and cornstarch were added, mixed vigorously, and then stored at ˜4° C.

TABLE 6
An exemplary formulation of a hand cream

| Ingredient | Amt | |
|---|---|---|
| Kelp water | ½ | cup |
| Soy lecithin | 4 | tbs |
| Shea butter | 1 | tsp |
| Sweet almond oil | 2 | tsp |
| Vanilla extract | 20 | drops |
| Lavender essential oil | 4 | drops |
| Non-hydrolyzed silk fibroin | 2 | tsp |
| Cornstarch | 2 | tsp |

The hand cream is lightweight and when used on the skin yields a moisturized, lustrous feel. For best results, hands can be washed before applying.

Hair Serum (Let You Hair Down Smoothing Serum).

An example of a hair serum formulation is shown in Table 7. The vegetable glycerin was added with the geranium and kiwi distillate. Non-hydrolyzed silk fibroin solution was then added and mixed gently until it was well mixed. The mixture was then stored at ˜4° C.

TABLE 7
An exemplary formulation of a hair serum

| Ingredient | Amt | |
|---|---|---|
| Non-hydrolyzed silk fibroin | 25 | ml |
| Vegetable glycerin | 100 | ml |
| Geranium (diluted 10x) | 1 | drop |
| Kiwi distillate | ½ | tsp |

The hair serum is applied to damp hair. A dime-size amount can be massaged hair strands and then blow-dried/styled as usual. The hair serum can also be applied to dry hair and used as a hair sculpting agent. The product moisturizes the hair and provides a non-greasy shine. When used as a styling agent, it holds hair in place without being stiff or sticky.

### REFERENCES

Carruthers, C. M. and Foster, P. M. D. (2005), Critical window of male reproductive tract development in rats following gestational exposure to di-n-butyl phthalate. Birth Defects Research Part B: Developmental and Reproductive Toxicology, 74: 277-285. doi: 10.1002/bdrb. 20050
Cavanagh, H. M. A. and Wilkinson, J. M. (2002), Biological activities of Lavender essential oil. Phytother. Res., 16: 301-308. doi: 10.1002/ptr.1103
Collins, Ben H., et al. “Kiwifruit protects against oxidative DNA damage in human cells and in vitro.” Nutrition and cancer 39.1 (2001): 148-153.
Darbre, P. D., Aljarrah, A., Miller, W. R., Coldham, N. G., Sauer, M. J. and Pope, G. S. (2004), Concentrations of parabens in human breast tumours. J. Appl. Toxicol., 24: 5-13. doi: 10.1002/jat.958
Dikstein, S. (1995), Hydropharmacology. Cell Biochem. Funct., 13: 195-200. doi: 10.1002/cbf.290130310
Hu X, Kaplan D, Cebe P. Determining beta-sheet crystallinity in fibrous proteins by thermal analysis and infrared spectroscopy. Macromolecules 2006; 39:6161-6170
Thiele, J. J., Hsieh, S, N. and Ekanayake-Mudiyanselage, S. (2005), Vitamin E: Critical Review of Its Current Use in Cosmetic and Clinical Dermatology. Dermatologic Surgery, 31: 805-813. doi: 10.1111/j.1524-4725.2005.31724
Vincent Vagenende, Miranda G. S. Yap, and Bernhardt L. Trout Biochemistry 2009 48 (46), 11084-11096
Yucel T, Kojic N, Leisk G G, Lo T J, Kaplan D L Non-equilibrium silk fibroin adhesives. J Struct Biol. 2010 May; 170(2):406-12. doi: 10.1016/j.jsb.2009.12.012. Epub 2009 Dec. 21.

All patents and other publications identified in the specification and examples are expressly incorporated herein by reference for all purposes. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A silk fibroin-based composition comprising non-hydrolyzed silk fibroin and a humectant agent.

2. The silk fibroin-based composition of claim 1, wherein the non-hydrolyzed silk fibroin has an average molecular weight of at least about 20 kDa or higher.

3. The silk fibroin-based composition of any of claims 1-3, wherein the non-hydrolyzed silk fibroin and humectant agent are present in a volume ratio of about 1:2 to about 1:100.

4. The silk fibroin-based composition of any of claims 1-4, wherein the non-hydrolyzed silk fibroin and humectant agent are present in a volume ratio of about 1:2 to about 1:10.

5. The silk fibroin-based composition of any of claims 1-5, wherein the non-hydrolyzed silk fibroin is present in an amount of about 1% (w/v or w/w) to about 30% (w/v or w/w).

6. The silk fibroin-based composition of any of claims 1-6, wherein the non-hydrolyzed silk fibroin is present in an amount of about 1% (w/v or w/w) to about 10% (w/v or w/w).

7. The silk fibroin-based composition of any of claims 1-7, wherein the humectant agent is selected from the group consisting of phospholipid (e.g., lecithin, panthenol (pro-vitamin B5), glycerin (glycerol)), aloe vera, sorbitol, urea, alpha-hydroxy acids, sodium pyroglutamate, N-acetyl-ethanolamine, sodium lactate, isopropanol, polyalkylene glycols (e.g., ethylene glycol, propylene glycol, hexylene glycol, butylene glycol, dipropylene glycol, triethylene glycol), 1,3-propanediol, polyethylene glycols, synthetic alcohols (e.g., glyceryl coconate, hydroxystearate, myristate, oleate), and any combinations thereof.

8. The silk fibroin-based composition of any of claims 1-8, wherein the humectant agent comprises phospholipid (e.g., lecithin, panthenol (pro-vitamin B5), glycerin), aloe vera, sorbital, alpha-hydroxy acids, or any combinations thereof.

9. The silk fibroin-based composition of any of claims 1-8, wherein the humectant agent comprises glycerin (glycerol).

10. The silk fibroin-based composition of claim 9, wherein the glycerin is present in an amount of about 50% (w/v or w/w) to about 99% (w/v or w/w).

11. The silk fibroin-based composition of any of claims 1-10, wherein the composition is a gel or hydrogel, a paste, a lotion, a cream, an ointment, an oil, a liquid, a serum, a shampoo, a foam or mousse, a spray, an aerosol, a scrub, or any combinations thereof.

12. The silk fibroin-based composition of any of claims 1-11, wherein the composition is translucent.

13. The silk fibroin-based composition of any of claims 1-12, wherein the composition is flowable.

14. The silk fibroin-based composition of any of claims 1-13, wherein the composition does not precipitate when exposed to shear.

15. The silk fibroin-based composition of any of claims 1-14, wherein the composition has a larger amount of silk fibroin in random coil conformation, as compared to a composition comprising the non-hydrolyzed silk fibroin in the absence of the humectant agent.

16. The silk fibroin-based composition of any of claims 1-15, wherein the composition has a smaller amount of silk fibroin in beta-sheet conformation, as compared to a composition comprising the non-hydrolyzed silk fibroin in the absence of the humectant agent.

17. The silk fibroin-based composition of any of claims 1-16, wherein the composition does not have a peak in the amide II region (between ˜1475 cm⁻¹ and ˜1560 cm⁻¹), as determined in a Fourier Transform Infrared Spectroscopy (FTIR) spectrum.

18. The silk fibroin-based composition of any of claims 1-17, further comprising an additive.

19. The silk fibroin-based composition of claim 18, wherein the additive comprises aqueous-soluble components, oil-soluble components, wax-soluble components, essential oils, emulsifiers, surfactants, flavors, fragrance, nutraceuticals, vitamins, therapeutic agents, cosmeceuticals, preservatives, botanical extracts or distillates, antioxidants, anti-bacterial agents, anti-inflammatory agents, cosmetically-acceptable agents, skin-lightening agents, skin-evening agents, anti-reddening agents, viscosity modifiers, emollients, or any combinations thereof.

20. The silk fibroin-based composition of any of claims 1-19, wherein the composition is formulated for use in a personal care product (e.g., a cosmetic, skincare, body care, or hair product).

21. The silk fibroin-based composition of any of claims 1-20, wherein the composition is formulated for use in a food product.

22. The silk fibroin-based composition of any of claims 1-21, wherein the composition is stable (e.g., no brittle gel formation) for at least about 1 month at room temperature.

23. A composition comprising an aqueous-based phase, an oil-based phase or a wax-based phase, and an emulsifier, wherein the aqueous-based phase comprises non-hydrolyzed silk fibroin and a humectant agent.

24. The composition of claim 23, wherein the non-hydrolyzed silk fibroin has an average molecular weight of at least about 20 kDa or higher.

25. The composition of any of claims 23-24, wherein the non-hydrolyzed silk fibroin and humectant agent are present in a volume ratio of about 1:2 to about 1:100 in the aqueous-based phase.

26. The composition of any of claims 23-25, wherein the non-hydrolyzed silk fibroin and humectant agent are present in a volume ratio of about 1:2 to about 1:10 in the aqueous-based phase.

27. The composition of any of claims 23-26, wherein the non-hydrolyzed silk fibroin is present in an amount of about 1% (w/v or w/w) to about 30% (w/v or w/w) of the aqueous phase.

28. The composition of any of claims 23-27, wherein the non-hydrolyzed silk fibroin is present in an amount of about 1% (w/v or w/w) to about 10% (w/v or w/w) of the aqueous phase.

29. The composition of any of claims 23-28, wherein the humectant agent is selected from the group consisting of phospholipid (e.g., lecithin, panthenol (pro-vitamin B5), glycerin (glycerol)), aloe vera, sorbitol, urea, alpha-hydroxy acids, sodium pyroglutamate, N-acetyl-ethanolamine, sodium lactate, isopropanol, polyalkylene glycols (e.g., ethylene glycol, propylene glycol, hexylene glycol, butylene glycol, dipropylene glycol, triethylene glycol), 1,3-propanediol, polyethylene glycols, synthetic alcohols (e.g., glyceryl coconate, hydroxystearate, myristate, oleate), and any combinations thereof.

30. The composition of any of claims 23-29, wherein the humectant agent comprises phospholipid (e.g., lecithin, panthenol (pro-vitamin B5), glycerin), aloe vera, sorbital, alpha-hydroxy acids, or any combinations thereof.

31. The composition of any of claims 23-28, wherein the humectant agent comprises glycerin (glycerol).

32. The composition of claim 31, wherein the humectant agent is present in an amount of about 50% (w/v or w/w) to about 99% (w/v or w/w) in the aqueous-based phase.

33. The composition of any of claims 23-32, wherein the oil-based phase comprises a carrier oil.

34. The composition of claim 33, wherein the carrier oil is a synthetic or natural oil derived from seeds, nuts, fruits, flowers, plant-based materials, or any combinations thereof.

35. The composition of any of claims 23-32, wherein the wax-based phase comprises butters, wax, paraffin wax, paraffin oil, petrolatum, or any combinations thereof.

36. The composition of any of claims 23-35, wherein at least one or both of the aqueous-based phase and the oil-based phase or the wax-based phase further comprise an additive.

37. The composition of claim 36, wherein the additive comprises aqueous-soluble components, oil-soluble components, wax-soluble components, essential oils, surfactants, flavors, fragrance, nutraceuticals, vitamins, therapeutic agents, preservatives, botanical extracts or distillates, antioxidants, anti-bacterial agents, anti-inflammatory agents, cosmetically-acceptable agents, cosmeceuticals, skin-lightening agents, skin-evening agents, anti-reddening agents, viscosity modifiers, emollients, or any combinations thereof.

38. The composition of any of claims 23-37, wherein the ratio of the aqueous-based phase to the oil-based phase is about 1:100 to about 100:1.

39. The composition of any of claims 23-38, wherein the emulsifier is selected from the group consisting of liquid soy lecithin, solid soy lecithin, honey, beeswax, cetyl alcohol, or any combinations thereof.

40. The composition of any of claims 23-39, wherein the composition is a gel or hydrogel, a paste, a lotion, a cream, an ointment, an oil, a liquid, a serum, a shampoo, a foam or mousse, a spray, an aerosol, a stick, a balm, a bar, a scrub, or any combinations thereof.

41. The composition of any of claims 23-40, wherein the composition is translucent.

42. The composition of any of claims 23-41, wherein the composition is flowable.

43. The composition of any of claims 23-42, wherein the composition does not precipitate when exposed to shear.

44. The composition of any of claims 23-43, wherein the composition is formulated for use in a personal care product (e.g., a cosmetic, skincare, body care, or hair product).

45. The composition of any of claims 23-44, wherein the composition is formulated for use in a food product.

46. A method of producing a silk fibroin-based composition comprising:
mixing a non-hydrolyzed silk fibroin solution and a humectant agent in a volume ratio of about 1:2 to about 1:100.

47. The method of claim 46, wherein the volume ratio of the non-hydrolyzed silk fibroin solution to the humectant agent is about 1:2 to about 1:10.

48. The method of claim 46 or 47, wherein the humectant agent is selected from the group consisting of phospholipid (e.g., lecithin, panthenol (pro-vitamin B5), glycerin (glycerol)), aloe vera, sorbitol, urea, alpha-hydroxy acids, sodium pyroglutamate, N-acetyl-ethanolamine, sodium lactate, isopropanol, polyalkylene glycols (e.g., ethylene glycol, propylene glycol, hexylene glycol, butylene glycol, dipropylene glycol, triethylene glycol), 1,3-propanediol, polyethylene glycols, synthetic alcohols (e.g., glyceryl coconate, hydroxystearate, myristate, oleate), and any combinations thereof.

49. The method of any of claims 46-48, wherein the humectant agent comprises phospholipid (e.g., lecithin, panthenol (pro-vitamin B5), glycerin), aloe vera, sorbital, alpha-hydroxy acids, or any combinations thereof.

50. The method of any of claims 46-49, wherein the non-hydrolyzed silk fibroin solution has a concentration of about 1% (w/v or w/w) to about 30% (w/v or w/w).

51. The method of any of claims 46-50, wherein the non-hydrolyzed silk fibroin solution has a concentration of about 1% (w/v or w/w) to about 10% (w/v or w/w).

52. The method of any of claims 46-51, further comprising mixing an additive with the humectant agent and the non-hydrolyzed silk fibroin solution.

53. The method of claim 52, wherein the additive is mixed with the humectant agent prior to said mixing with the non-hydrolyzed silk fibroin solution.

54. The method of any of claims 46-53, further comprising mixing an aqueous mixture comprising the non-hydrolyzed silk fibroin solution and the humectant agent with an oil-based phase or a wax-based phase.

55. The method of claim 54, wherein the oil-based phase comprises a carrier oil and an emulsifier.

56. The method of claim 55, wherein the carrier oil is a synthetic or natural oil derived from seeds, nuts, fruits, flowers, plant-based materials, or any combinations thereof.

57. The method of claim 54, wherein the wax-based phase comprises butters, wax, paraffin wax, paraffin oil, petrolatum, or any combinations thereof.

58. The method of any of claims 55-57, wherein the emulsifier is selected from the group consisting of liquid soy lecithin, solid soy lecithin, honey, beeswax, cetyl alcohol, or any combinations thereof.

59. The method of any of claims 55-58, wherein the volume ratio of the carrier oil to the emulsifier is about 2:1 to about 100:1.

60. The method of any of claims 54-59, wherein the oil-based phase further comprises an additive.

61. The method of claim 52 or 60, wherein the aqueous-soluble or the oil-soluble additive is selected from the group consisting of essential oils, flavors, fragrance, nutraceuticals, therapeutic agents, preservatives, botanical extracts or distillates, antioxidants, anti-bacterial agents, anti-inflammatory agents, cosmetically-acceptable agents, viscosity modifiers, emollients, or any combinations thereof.

62. A composition produced by the method of any of claims 46-61.

63. The composition of claim 62, wherein the composition is formulated for use in a personal care product (e.g., a cosmetic, skincare, body care, or hair product).

64. The composition of claim 62, wherein the composition is formulated for use in a food product.

65. A method comprising:
applying on a target surface a composition of any of claim 1-45 or 62-64.

66. The method of claim 65, wherein the applying comprises rubbing.

67. The method of claim 65 or 66, wherein the target surface is skin or hair of a subject.

68. The method of any of claims 65-67, further comprising maintaining the composition at room temperature for at least about 1 month, wherein the composition remains stable thereafter.
